(19) United States
(12) Patent Application Publication
(10) Pub. No.: US 2023/0077716 A1
(43) Pub. Date: Mar. 16, 2023

(54) ANTIBODIES PROTECTIVE AGAINST INFLUENZA B

(71) Applicant: Washington University, St. Louis, MO (US)

(72) Inventor: Ali Ellebedy, St. Louis, MO (US)

(21) Appl. No.: 17/798,261

(22) PCT Filed: Feb. 5, 2021
(86) PCT No.: PCT/US2021/016879

§ 371 Date: Aug. 8, 2022

Related U.S. Application Data
(60) Provisional application No. 62/971,671, filed on Feb. 7, 2020.

(51) Int. Cl. C07K 16/10; A61P 31/16

(52) U.S. Cl. CPC C07K 16/1018; A61P 31/16; C07K 2317/565; C07K 2317/21; C07K 2317/76; C07K 2317/33; C07K 2317/34

(57) ABSTRACT

The present invention relates to antibodies or antigen-binding fragments that are useful for treating influenza B viruses. The present invention also relates to various pharmaceutical compositions and methods of treating influenza using the antibodies or antigen-binding fragments.

### STATEMENT REGARDING FEDERALLY SPONSORED RESEARCH OR DEVELOPMENT

This invention was made with government support under AI141990, AI139813 and HHSN272201400006C awarded by the National Institutes of Health. The government has certain rights in the invention.

### REFERENCE TO MATERIAL INCORPORATED BY REFERENCE

The instant application contains a Sequence Listing as a text file, which is entitled “GENE SEQUENCE LISTING” as created on Feb. 4, 2021, and is 64,796 bytes in size. This sequence listing was submitted via EFS-Web in ASCII format on Feb. 5, 2021, and is hereby incorporated by reference in its entirety.

### FIELD OF THE INVENTION

The present invention relates to antibodies or antigen-binding fragments that are useful for treating influenza B viruses. The present invention also relates to various pharmaceutical compositions and methods of treating influenza using the antibodies or antigen-binding fragments.

### BACKGROUND

Seasonal influenza virus infections result in significant global morbidity and mortality. Influenza B virus (IBV) infection is the cause of approximately 25% of all influenza cases (Paul Glezen et al., 2013; Tan et al., 2018). Circulating IBVs are phylogenetically divided into two distinct lineages based on their hemagglutinin (HA) sequences, B/Yamagata/16/88-like (Y) and B/Victoria/2/87-like (V) (Rota et al., 1990). Recently, the B/Yamagata/16/88-like lineage further split into clades 2 and 3 alongside B/Victoria/2/87-like viruses with 1, 2 or 3 amino acid deletion mutants have emerging, which adds to antigenic diversity (Langat et al., 2017; Virk et al., 2019). Current quadrivalent seasonal influenza virus vaccines include representative strains from both IBV lineages in addition to the two circulating influenza A strains from the H1N1 and H3N2 subtypes. These vaccines work mainly by eliciting an antibody response directed against the major surface glycoprotein of the virus, HA (Ellebedy and Ahmed, 2012; Krammer, 2019). Vaccine-induced antibody responses can be rendered largely ineffective by the continuous antigenic drifting of circulating influenza viruses, which significantly undermines overall vaccine effectiveness. Consequently, strains to be included in seasonal vaccines need to be reviewed on a biannual basis, creating an urgent need for new vaccines and treatment options that can provide broader and more durable protection against the ever-evolving influenza viruses (Ellebedy and Webby, 2009).

Neuraminidase (NA) is the second major surface protein on the influenza virus (Krammer et al., 2018). NA functions by cleaving terminal sialic acid residues from N-linked glycans, leading to the release of virus trapped by natural defense proteins like mucins and, importantly, facilitates egress of virus from infected cells. Antibodies directed against NA can block influenza virus replication mainly by interfering with viral egress (Eichelberger et al., 2018). Both anti-NA monoclonal antibodies (mAbs) and NA vaccination-induced polyclonal antibodies protect against lethal influenza virus challenge in animal models (Stadlbauer et al., 2018; Wohlbold et al., 2015). Moreover, mucosal anti-influenza BNA antibodies can prevent viral transmission in guinea pigs (McMahon et al., 2019). In addition to antibodies, NA is the target of oseltamivir, the most widely prescribed anti-influenza antiviral drug (Govorkova and McCullers, 2013). Importantly, oseltamivir is currently the only anti-influenza antiviral drug approved by the U.S. Food and Drug Administration (FDA) for use in children 2 years of age and younger (Burnham et al., 2013). However, it is well established that oseltamivir is less effective for influenza B than for influenza A infection with regard to the duration of fever and virus persistence especially in the pediatric population (Kawai et al., 2006; Sato et al., 2008; Sugaya et al., 2007).

Broadly protective anti-influenza B virus NA antibodies have recently been described (Piepenbrink et al., 2019; Wohlbold et al., 2017). Our group has recently reported the isolation and characterization of three clonally related monoclonal antibodies (mAbs) derived from plasmablasts isolated from an H3N2-infected individual that show broad, heterosubtypic NA inhibition activity against influenza A virus group 1 and group 2 NA expressing strains, as well as a fraction of influenza B viruses (Stadlbauer et al., 2019). These antibodies target conserved residues within the NA active site.

There remains a need for broadly protective antibodies to Influenza B viruses.

### BRIEF SUMMARY

Aspects of the present invention relate to various antibodies or antigen-binding fragments thereof. In various embodiments, antibodies or antigen-binding fragments thereof comprise: (a) an immunoglobulin heavy chain variable region comprising an amino acid sequence having at least 70% identity to any one of SEQ ID NOs: 1-21 and 93-99; or (b) an immunoglobulin light chain variable region comprising an amino acid sequence having at least 70% identity to any one of SEQ ID NOs: 22-41 and 100-106.

Other embodiments include various antibodies or antigen-binding fragments having affinity for an influenza B virus. These antibodies or antigen-binding fragments bind to an active site residue of a neuraminidase expressed on the surface of the virus, wherein the active site residue comprises at least one residue selected from: R147, K435, R116, R292, R374, Y409, D149, E226, H134, R147, R116, and R374 according to the amino acid numbering of SEQ ID NO: 149.

Further aspects of the present invention relate to nucleic acids comprising a nucleotide sequence encoding an immunoglobulin light chain variable region and/or an immunoglobulin heavy chain variable region of any antibody or antigen-binding fragment as described herein. Other aspects of the present invention relate to expression vectors comprising the nucleic acids, host cells comprising the expression vectors as well as methods of producing the antibodies and antigen-binding fragments thereof as described herein.

Still further aspects of the present invention relate to influenza vaccines. In some embodiments, the vaccines comprise a polypeptide comprising an amino acid sequence comprising at least about 70% identity to an epitope targeted by any antibody or antigen-binding fragment thereof described herein.

Further aspects relate to various pharmaceutical compositions comprising any of the antibodies or antigen-binding fragments thereof as described herein.

Additional aspects of the present invention relate to methods of preventing or treating influenza in a subject in need thereof. In various embodiments, the methods comprise administering to the subject any antibody or antigen-binding fragment thereof as described herein, any nucleic acid comprising a nucleotide sequence encoding at least a portion of an antibody or antigen-binding fragment thereof as described herein, any expression vector as described herein, any vaccine as described herein, or any composition comprising at least one of the antibodies or antigen-binding fragments thereof described herein.

Other objects and features will be in part apparent and in part pointed out hereinafter.

### BRIEF DESCRIPTION OF THE DRAWINGS

FIG. 1: Broadly cross-reactive anti-NA monoclonal antibodies. (A) A pie chart showing the specificity of the 21 recombinant human IBV-specific monoclonal antibodies (mAbs) derived from the IBV-infected patient's plasmablasts. The table on the right lists the IDs of the seven anti-NA mAbs (BNA-mAbs), their encoding heavy and light chain variable genes and the amino acid sequence of the heavy chain CDR3 for each mAb. (B) Binding by ELISA of the seven BNA-mAbs plus an IBV HA-specific mAb to recombinant NA molecules from six IBV strains. Binding to IBV HA is shown as a control.

FIG. 1.1: Broadly cross-reactive anti-NA monoclonal antibodies (Related to FIG. 1 and FIG. 2). (A) Frequencies of IgG+ plasmablasts specific for the indicated HA in freshly isolated PBMCs from the infected patient measured by ELIspot. Data from 1 experiment. (B) Binding by ELISA of 1G01 to recombinant NA molecules from three IBV strains. Data representative of 2 experiments. (C) Binding overlap among BNA-mAbs by competition ELISA. Percent competition for each mAb was calculated as the reduction in binding relative to the b against itself. Data representative of 2 (B) or from 1 (A, C) experiment.

FIG. 1.2: Table of immunoglobin gene usage of BNA-mAbs.

FIG. 2: BNA-mAbs exhibit broadly cross-reactive virus inhibition and neutralization in vitro. (A) NA inhibition (NI) IC50 of BNA-mAbs against the indicated IBV strains measured by ELLA. IAV NA-specific mAb 1G01 and irrelevant human IgG1 are negative controls. See also FIG. 2.1. (B) NI of BNA-mAbs against B/Phuket/3073/13 (Y) in an NA-Star assay. Symbols represent mean±SD. (C) Neutralization capacity of BNA-mAbs against B/Phuket/3073/13 (Y) and B/Brisbane/60/08 (V) measured by plaque reduction assay. See also FIG. 3.1. Data are representative of two experiments. See also FIGS. 1.1, 2.1, 3.1.

FIG. 2.1: BNA-mAbs exhibit broadly cross-reactive virus inhibition activity (Related to FIG. 2). (A-I) NI cur in ELLA assays. (J-M) NI curves 1G05 and 2E01 against wild-type (J, L) and oseltamivir-resistant (K LLA assays. Symbols represent mean±SD. Data representative of 2 experiments.

FIG. 3: BNA-mAbs are broadly protective in vivo. Protective efficacy of the BNA-mAbs in a mouse model against challenge with two IBV strains. (A and B) Animals were injected with each of the mAbs (5 mg/kg) intraperitoneally two hours before the intranasal virus challenge with B/New York/PV00094/17 (Y). Five animals per mAb were used. (C and D) Lung titers of animals treated prophylactically with mAbs [as described for (A)] on day 3 and day 6 after infection. Three mice per group were used. (E and F) Protective efficacy of the BNA-mAbs against B/New York/PV01181/18 (V). Same experimental setup as described for (A). (G and H) Protective capacity of the BNA-mAbs as tested in a therapeutic setting. 5 mg/kg of each of the mAbs were injected 72 hours after infection with B/New York/PV00094/17 (Y). Five animals were used per group. For (A), (C) and (G), survival is indicated. For (B), (F) and (H), percent weight loss is shown. Weight loss was monitored daily.

FIG. 3.1: BNA-mAbs exhibit protection in vitro and in vivo (Related to FIG. 2 and FIG. 3). (A-B) Virus ne B/Phuket/3073/13 (Y) and B/Brisbane/60/08 (V) as measured by plaque reduction assay. (C-D) ADCC activity of BNA-mAbs against B/Phuket/3073/13 (Y) and B/Brisbane/60/08 (V) as measured by ADCC bioreporter assay. (E-F) Survival (E) and percent original weight (F) of mice challenged with B/New York/PV000/17 (Y) 2 h after administration of the indicated mAb. Five mice per mAb were used. Symbols represent mean±SD. * P<0.05, Mantel-Cox log rank test between each mAb and isotype. (G-I) Binding of BNA-mAbs 1G05 and 2E01 and broadly binding mAb 1G01 to the indicated IAV (G, H) and I As. A-D, G-I, Data representative of 2 experiments. Symbols represent mean±SD.

FIG. 4: Cryo-EM reconstruction of NA-1G05 and NA-2E01 particles. (A) Reference model-free 2D-classification of NA-1G05. (B) 2D-classification of NA-2E01. (C) Fourier Shell Correlation (FSC) curves after post-process with Relion-3. (D) FSC curves of NA-2E01. (E) Cryo-EM reconstruction of Fab 1G05 in complex with NA at 2.5 Å resolution. It is shown that one NA tetramer bound with four Fabs. (F) Cryo-EM reconstruction of Fab 2E01 in complex with NA at 2.8 Å resolution.

FIG. 4.1: Comparison of 1G05 and 2E01 with inferred germline ancestors (Related to FIG. 4). (A, B) Alignment of the amino acid sequences of 1G05 (A) and 2E01 (B) heavy chain (top) and light chain (bottom) to their inferred germline immunoglobulin genes. (C-F) Quantitative analysis of Fabs tors (E, F) binding to B/Phuket/3073/13 NA by BLI.

FIG. 5: Atomic models of NA-1G05 complex and NA-2E01 complex. (A) Local resolution of the map of one monomeric subunit of NA bound with one 1G05 molecule. (B) Local resolution of the map of one NA protomer bound with one 2E01 molecule. (C) Ribbon diagram of one monomeric subunit of NA bound with 1G05, in the same orientation as in (A). NA is shown in gray, the mAb heavy chain is in cyan and the mAb light chain is in teal. N-linked glycan moieties are shown as sticks in yellow. (D) Ribbon diagram of NA protomer bound with 2E01, in the same orientation as in (B). NA is shown in gray, the mAb heavy chain is in green and the mAb light chain is in dark green. N-linked glycan moieties are shown as sticks in yellow. (E) Electron density map and atomic model of 1G05 CDR-H3 (contour level at 5.0 σ). (F) Electron density map and atomic model of 2E01 CDR-H3 (contour level at 5.0σ).

FIG. 5.1: Local resolution analyses of NA-Fabs reconstruction and interface (Related to FIG. 5). (A, B) Local resolutions of NA-1G05 (A) and NA-2E01 (B) reconstructions. (C, D) Atomic models of NA-1G05 (C) and NA-2E01 (D) tetramers. (E, F) Local resolution analysis of NA-Fabs interface. (E) In NA-1G05, the resolution of the paratope is within 2.6 Å, while the resolution of H3 and the active site of NA are within 2.5 Å. (F) In NA-2E01, the resolution of the paratope is within 3.0 Å, while H3 is within 2.8 Å and the active site of NA are within 3 Å.

FIG. 6: Epitope analysis of 1G05 and 2E01. (A) The epitope to 1G05 HC is shown as a pink-colored surface. The 1G05 HC and LC are shown as spheres in cyan and teal, respectively. (B) Epitope residues making either polar or hydrophobic interactions via side chains with 1G05 are labeled in black. Crucial contacting residues on CDRs are shown as sticks in cyan. (C) Conservation analysis of epitope to 1G05 with amino acid sequences from all influenza B strains (upper panel), and all influenza B and influenza A strains (lower panel) tested in the paper. (D) The epitope of 2E01 HC and LC is shown as pink- and dark pink-colored surfaces, respectively. The 2E01 HC and LC are shown as spheres in green and dark green. (E) Epitope residues making polar interactions via side chains with 2E01 are labeled in black. Crucial contacting residues on CDRs are shown as sticks in green and dark green, respectively. (F) Conservation analysis of epitope to 2E01 with amino acid sequences from all influenza B strains (left panel), and all influenza B and influenza A strains (right panel) tested in the paper.

FIG. 6.1: Interactions of 1G05 and 2E01 CDRs with NA (Related to FIG. 5 and FIG. 6). (A-D) Interaction of 1G05 CDRs with NA. (E-G) Interaction of 2E01 CDRs with NA.

FIG. 7: Comparison of NA active site blocked by H3 from Fabs and oseltamivir. (A) Close-up view of the interaction between NA and 1G05. H3 is shown as cartoon loops in cyan with crucial interacting residues shown as sticks. Residues on NA are shown as sticks in gray. Polar interactions are shown with dashed lines. (B) Interactions between NA and 2E01 in the same orientation as (A). H3 is shown as cartoon loops in green with crucial interacting residues shown as sticks. Residues on NA are shown as sticks in gray. Polar interactions are shown with dashed lines. (C) Interaction of sialic acid and NA from B/Beijing/1/1987 virus (PDB ID 1NSC). Sialic acid is shown as orange sticks. Residues on NA shown as gray sticks. Polar interactions are shown as dashed lines. (D) Interaction of oseltamivir and NA from B/Brisbane/60/2008 virus (PDB ID 4CPM). Oseltamivir is shown as sticks in blue. Residues on NA are shown as sticks in gray. Polar interactions are shown with dashed lines.

FIG. 7.1: Conservation of key residues in epitopes recognized by 1G05 and 2E01 among NAs (Related to FIG. 6 and FIG. 7). (A) Amino acid alignment of NAs of IBV and IAV strains used in the study. Each epitope residue is labeled with the total number of contacts if it makes van der Waals contact within 3.90-Å distance with Fab 1G05 or 2E01. Crucial epitope residues for 1G05 labeled in FIG. 6, B are indicated in cyan triangles. Crucial epitope residues for 2E01 labeled in FIG. 6, E are indicated in green triangles. (B) Primary sequence alignment of NAs form all influenza strains used in the study. Each epitope residue is labeled with the total number of contacts if it makes van der Waals contact within 3.90-Å distance with Fab 1G05 or 2E01. Crucial epitope residues for 2E01 labeled in FIG. 6, B are indicated in cyan triangles. Crucial epitope residues for 2E01 labeled in FIG. 6, E are indicated in green triangles. Catalytic residues on NA are indicated with stars.

FIG. 8: General structure of an IgG antibody.

### DETAILED DESCRIPTION

Aspects of the present invention relates to various antibodies and antigen-binding fragments thereof that show specificity to influenza B viruses. Antibodies and antigen-binding fragments thereof described herein can neutralize the virus, mediate effector functions, can be protective in vivo, and bind and inhibit NA activity in a similar mode to that of other NA inhibitors (e.g., oseltamivir) by blocking the active pocket using long CDR-H3 loops. In various embodiments, the antibodies and antigen-binding fragments can comprise an immunoglobulin heavy chain variable region comprising an amino acid sequence having at least 70% identity to any one of SEQ ID NOs: 1-21 and 93-99; or an immunoglobulin light chain variable region comprising an amino acid sequence having at least 70% identity to any one of SEQ ID NOs: 22-41 and 100-106. Specific light and heavy chains of various antibodies and antigen-binding fragments are described in more detail herein.

### Influenza Type B Specificity and Antibody Properties

Applicants have discovered highly active anti-NA antibodies that show high specificity for influenza B viruses. Accordingly, in various embodiments, the antibody or antigen-binding fragment thereof can selectively bind to an Influenza B virus. The antibodies and antigen-binding fragments described herein can have important applications, for both therapeutic and prophylactic treatment of influenza infections.

### Therapeutic Use to Manage Acute Infections

Neuraminidase is a validated drug target and several small molecules that inhibit its activity are licensed as influenza therapeutics. Like the mAbs described herein, these small molecules target the active site of the NA. Therefore, and because of the extensive breadth of these mAbs, they could potentially be used as antivirals for treatment of seasonal, pandemic and zoonotic influenza virus infection in humans. While small molecules certainly have advantages, the therapeutic window of these drugs is restricted to 48 hours post-onset of symptoms. In the mouse model, our mAbs showed solid protection when administered as late as 72 hours following a lethal influenza virus challenge, suggesting that they might have a longer therapeutic window. The basis for this effect might be their strong IBV NA activity combined with effector function and potentially modulation of the immune response to infection (30).

In summary, mAbs were synthesized that are clonally related and bind to the influenza type B neuraminidase by inserting a long CDR H3 into the enzymatic active site, taking up the space usually occupied by sialic acid. The mAbs show broad binding across different strains of influenza B viruses, making these mAbs suitable for therapeutic development. These antibodies are highly active inhibitors of NA activity in vitro and provide broad protection from mortality and morbidity in vivo. The discovery of these mAbs raises the hope that similar antibodies can be induced in the population if the right vaccination regimen is given. Knowledge about the binding mode and epitope of these mAbs may then guide the development of NA-based universal influenza B virus vaccines.

### Antibody Structure and Sequences Thereof

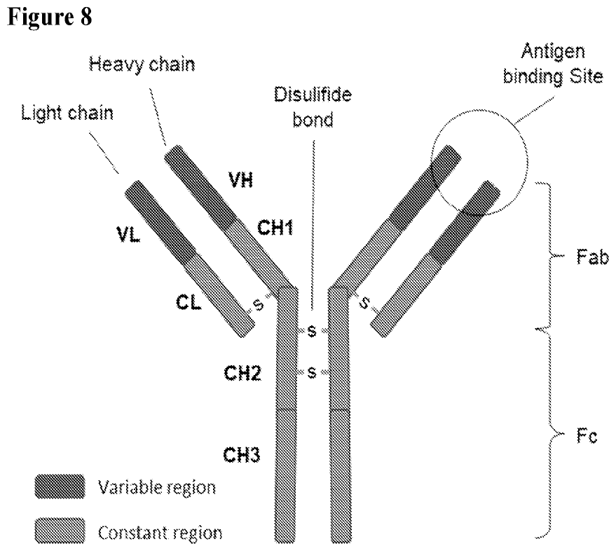

The general structure of an IgG antibody is shown in FIG. 8. Briefly, there are two major subunits: the heavy chain and the light chain connected via disulfide bonds. Each heavy chain and light chain is further divided into a variable or a constant region. The variable regions interact most directly with the antigen and further comprise three hyper variable regions (complementary determining domains, CDRs). Thus, a single antibody comprising two heavy chains and two light chains comprises a total of twelve CDRs (three for each heavy chain and each light chain). However, each of the variable regions, particularly the CDRs, possess some degree of affinity for the antigen and maximum affinity can be achieved with a single heavy chain coupled to a single light chain. For this reason, a typical IgG antibody is considered divalent and can potentially target two different antigens simultaneously depending on the identity of the heavy and light chains. The variable region of the antibody (both the heavy and light chains) is collectively known as the Fab fragment and can be cleaved from the constant region (known as the Fc portion) to form an antigen-binding fragment. In addition, as noted each of the CDRs possess some degree of affinity for the antigen, and can each be considered an antigen-binding fragment. An antibody fragment can have an equivalent binding affinity for the target as the parent antibody. Both divalent and monovalent antibody fragments are included in the present invention.

Therefore, in various embodiments, the antibody or antibody binding fragment comprises a heavy chain variable region (or fragment thereof) and/or a light chain variable region (or fragment thereof). The heavy chain variable region comprises three complementary defining regions (CDRs) classified as CDR_{H1}, CDR_{H2}, and CDR_{H3}. Likewise, the light chain variable region comprises three complementarity determining regions (CDRs) classified as CDR_{L1}, CDR_{L2}, and CDR_{L3}.

For ease of reference, illustrative CDRs of the antibodies of the present invention are shown below in Table 1.

TABLE 1
Illustrative CDR sequences for
Anti-IBV NA antibodies

| Complementary | | SEQ | Antibody |
|---|---|---|---|
| Determining | AMINO ACID | ID | of |
| Region | SEQUENCE | NO: | Origin |
| CDR_{H1} | GDSIGGSY | 1 | 1G05 |
| | GYTFINHA | 2 | 2E01 |
| | GGSISSGGNY | 3 | 1A03 |
| | GDSISGSSYY | 4 | 2D10 |
| | GFSFSAYG | 5 | 1D05 |
| | GVTLDNYW | 6 | 2H09 |
| | GFPFSHYY | 7 | 3C01 |
| CDR_{H2} | IYYTGIT | 8 | 1G05 |
| | IIPIFGLA | 9 | 2E01 |
| | ISYSGST | 10 | 1A03 |
| | IYYSGIT | 11 | 2D10 |
| | LGYDGTDQ | 12 | 1D05 |
| | INADGTSK | 13 | 2H09 |
| | IYSDGTSS | 14 | 3C01 |
| CDR_{H3} | ARGDYSGYDRDVQ | 15 | 1G05 |
| | VELMDV | | |
| | ARDTVAVYEDFD | 16 | 2E01 |
| | WSSPYFFYMDV | | |
| | ARGRGYCSRGAT | 17 | 1A03 |
| | CYNFYMDV | | |
| | ARLYTKSSNANY | 18 | 2D10 |
| | ARGARPYYTDYR | 19 | 1D05 |
| | DHRPSYFYYHMDV | | |
| | ARGGLYSSDAFDV | 20 | 2H09 |
| | CRGGYYSLDGFDF | 21 | 3C01 |
| CDR_{L1} | QTISIF | 22 | 1G05 |
| | QSAGSKS | 23 | 2E01 |
| | SGDIGGYNL | 24 | 1A03 |
| | QSISSW | 25 | 2D10 |
| | SSNIGNNY | 26 | 1D05 |
| | SWDVGRYNH | 27 | 2H09 |
| | QGIGND | 28 | 3C01 |
| CDR_{L2} | AAS | 29 | 1G05 |
| | GAS | 30 | 2E01, |
| | | | 3C01 |
| | EDS | 31 | 1A03 |
| | DAS | 32 | 2D10 |
| | DSD | 33 | 1D05 |
| | EVN | 34 | 2H09 |
| CDR_{L3} | QQSYSAPWT | 35 | 1G05 |
| | QRYGTSLVT | 36 | 2E01 |
| | CSHAGSVV | 37 | 1A03 |
| | QQYHSYSGT | 38 | 2D10 |
| | GTWDNSLNVLV | 39 | 1D05 |
| | SSYTGNNVAV | 40 | 2H09 |
| | LQHSSFPYT | 41 | 3C01 |

The CDRs are spaced out along the light and heavy chains and are flanked by four relatively conserved regions known as framework regions (FRs). Thus, the heavy chain variable region comprises four framework regions (FRs) classified as FR_{H1}, FR_{H2}, FR_{H3}, and FR_{H4} and the light chain variable region comprises four framework regions (FRs) classified as FR_{L1}, FR_{L2}, FR_{L3}, and FR_{L4}. Illustrative sequences for the framework regions in the antibodies described herein are shown in Table 2 below.

TABLE 2
Illustrative FR sequences
for Anti-NA antibodies

| | | SEQ | |
|---|---|---|---|
| Framing | | ID | Anti- |
| Region | AMINO ACID SEQUENCE | NO: | body |
| FR_{H1} | QVQLQESGPGLVRPSETLSLTCTVS | 42 | 1G05 |
| | EVQLVQSGAEVKKPGSSVKVSCKAS | 43 | 2E01 |
| | QVQLQESGPGLVKPSQTLSLTCTVS | 44 | 1A03 |
| | QVQLQESGPGLVKPSETLSLTCTVS | 45 | 2D10 |
| | EVQLVESGGSVVQPGRSLRLSCAAF | 46 | 1D05 |
| | EVQLVESGGGLVQPGGSLRLSCAAS | 47 | 2HO9, |
| | | | 3C01 |
| FR_{H2} | WNWIRQPPGKGLQWIGY | 48 | 1G05 |
| | LSWVRQAPGQGLEWVGG | 49 | 2E01 |
| | WSWIRQHPGKGLEWIGF | 50 | 1A03 |
| | WGWIRQPPGKGLEWIGS | 51 | 2D10 |
| | MHWVRQAPGKGLEWVTL | 52 | 1D05 |
| | VHWVRQVPGKGLVWVSR | 53 | 2H09 |
| | MHWVRRAPGKGLVWVSR | 54 | 3C01 |
| FR_{H3} | NYNPSLKSRVTMSLDTSKN | 55 | 1G05 |
| | QISLKMDSVTAADTALYFC | | |
| | KYGQKFQDRVTITADESTK | 56 | 2E01 |
| | TAYMDLRSLRSDDTAVYYC | | |
| | YFTPSLNSRLTISVDTTNN | 57 | 1A03 |
| | HFSLKLSSVTGADTAVYYC | | |
| | YYNPSLKSRVTIYVDTSKN | 58 | 2D10 |
| | QFSLKLNSATAADTAVYYC | | |
| | WVAESVKGRFTVSRDNSRN | 59 | 1D05 |
| | TVILQMDSLRAEDTAVYFC | | |
| | TYADSVKGRFTISRDTTRNT | 60 | 2H09 |
| | LFLQMNSLRGDDTALYFC | | |
| | SYGDSVKGRFTISRDNAKNI | 61 | 3C01 |
| | LYLQMNSLRAEDSATYYC | | |
| FR_{H4} | WGKGTTVTVSS | 62 | 1G05, |
| | | | 2E01 |
| | WGVGTTVTVSS | 63 | 1A03 |
| | WGQGTLVTVSS | 64 | 2D10 |
| | WGAGTTVTVSS | 65 | 1D05 |
| | WGQGTMVTVSS | 66 | 2HO9, |
| | | | 3C01 |
| FR_{L1} | DIQMTQSPSSLSASVRDKVTFVCRAS | 67 | 1G05 |
| | EIVLTQSPATLSLFPGERATLSCRAS | 68 | 2E01 |
| | QSALTQPASVSGSPGQSITISCTGT | 69 | 1A03 |
| | DIQMTQSPSTLSASVGDRVTITCRAS | 70 | 2D10 |
| | QSVLTQPPSVSAAPGQKVTISCSGS | 71 | 1D05 |
| | QSALTQPPSASGSPGQSVTISCTGT | 72 | 2H09 |
| | DIQMTQSPLSLSVSEGDRVTITCRAS | 73 | 3C01 |
| FR_{L2} | LNWYQHKPGEAPKLLIY | 74 | 1G05 |
| | LAWYQHKVGQPPRLLIN | 75 | 2E01 |
| | VSWYQHHPGRVPKLIIY | 76 | 1A03 |
| | LAWYQQKPGKAPKLLIY | 77 | 2D10 |
| | VSWYQQLPGTAPKLLIY | 78 | 1D05 |
| | VSWYQHHPGKAHKLIIY | 79 | 2H09 |
| | LGWYQLKPGKAPKRLIY | 80 | 3C01 |
| FR_{L3} | RLQSGVPSRFSGSGSGTDFTLTI | 81 | 1G05 |
| | SGLQPEDFATYYC | | |
| | SRATGIPDRFSGSGSGPDFNLTI | 82 | 2E01 |
| | SRLEPEDFAVYYC | | |
| | KRPSGFLNRFSGSKSGNTASLTI | 83 | 1A03 |
| | SGLQAEDVADYYC | | |
| | SLESGVPSRFSGSGSGTEFTLTI | 84 | 2D10 |
| | SSLQSDDFAIYYC | | |
| | KRPSGIPARFSGSKSGASATLAI | 85 | 1D05 |
| | TGLQTGDEADYYC | | |
| | RRPSGVPDRFSGSKSANTASLTV | 86 | 2H09 |
| | SGLQAEDEADYYC | | |
| | SLQSGVPSRFSGSGSGTEFTLTI | 87 | 3C01 |
| | SSLQPEDFATYFC | | |
| FR_{L4} | FGQGTKVEIK | 88 | 1G05, |
| | | | 2D10, |
| | | | 3C01 |
| | FGGGTKVEIK | 89 | 2E01 |
| | FGGGTRLTVQ | 90 | 1A03 |
| | FGGGTKLAVL | 91 | 1D05 |
| | FGGGTKLTVL | 92 | 2H09 |

Any of the CDR_{H} regions may be combined with one or more of the FR_{H} sequences described above to form a heavy chain variable region. In various embodiments, suitable heavy chain variable regions can comprise any one of SEQ ID NOs: 93-99. Moreover, since many conservative substitutions may be envisioned by one of ordinary skill in the art, the antibody or antibody binding fragment can comprise a heavy chain variable region comprising at least about 70% sequence identity to any one of SEQ ID NOs: 93-99.

For example, the antibody or antibody binding fragment can comprise a heavy chain variable region comprising at least about 75% sequence identity to any one of SEQ ID NOs: 93-99.

For example, the antibody or antibody binding fragment can comprise a heavy chain variable region comprising at least about 80% sequence identity to any one of SEQ ID NOs: 93-99.

For example, the antibody or antibody binding fragment can comprise a heavy chain variable region comprising at least about 85% sequence identity to any one of SEQ ID NOs: 93-99.

For example, the antibody or antibody binding fragment can comprise a heavy chain variable region comprising at least about 90% sequence identity to any one of SEQ ID NOs: 93-99.

For example, the antibody or antibody binding fragment can comprise a heavy chain variable region comprising at least about 95% sequence identity to any one of SEQ ID NOs: 93-99.

For example, the antibody or antibody binding fragment can comprise a heavy chain variable region comprising at least about 96% sequence identity to any one of SEQ ID NOs: 93-99.

For example, the antibody or antibody binding fragment can comprise a heavy chain variable region comprising at least about 97% sequence identity to any one of SEQ ID NOs: 93-99.

For example, the antibody or antibody binding fragment can comprise a heavy chain variable region comprising at least about 98% sequence identity to any one of SEQ ID NOs: 93-99.

For example, the antibody or antibody binding fragment can comprise a heavy chain variable region comprising at least about 99% sequence identity to any one of SEQ ID NOs: 93-99.

For example, the antibody or antibody binding fragment can comprise a heavy chain variable region comprising at least about 99.5% sequence identity to any one of SEQ ID NOs: 93-99.

For example, the antibody or antibody binding fragment can comprise a heavy chain variable region comprising at least about 99.9% sequence identity to any one of SEQ ID NOs: 93-99.

Likewise, any of the CDR_{L} regions may be combined with one or more of the FR_{L} sequences described above to form a light chain variable region. In various embodiments, suitable light chain variable regions can comprise any one of SEQ ID NOs: 100-106. Moreover, since many conservative substitutions may be envisioned by one of ordinary skill in the art without affecting the activity of the antibody, the antibody or antibody binding fragment can comprise a light chain variable region comprising at least about 70% sequence identity of any one of SEQ ID NOs: 100-106.

For example, the antibody or antibody binding fragment can comprise a light chain variable region comprising at least about 75% sequence identity to any one of SEQ ID NOs: 100-106.

For example, the antibody or antibody binding fragment can comprise a light chain variable region comprising at least about 80% sequence identity to any one of SEQ ID NOs: 100-106.

For example, the antibody or antibody binding fragment can comprise a light chain variable region comprising at least about 85% sequence identity to any one of SEQ ID NOs: 100-106.

For example, the antibody or antibody binding fragment can comprise a light chain variable region comprising at least about 90% sequence identity to any one of SEQ ID NOs: 100-106.

For example, the antibody or antibody binding fragment can comprise a light chain variable region comprising at least about 95% sequence identity to any one of SEQ ID NOs: 100-106.

For example, the antibody or antibody binding fragment can comprise a light chain variable region comprising at least about 96% sequence identity to any one of SEQ ID NOs: 100-106.

For example, the antibody or antibody binding fragment can comprise a light chain variable region comprising at least about 97% sequence identity to any one of SEQ ID NOs: 100-106.

For example, the antibody or antibody binding fragment can comprise a light chain variable region comprising at least about 98% sequence identity to any one of SEQ ID NOs: 100-106.

For example, the antibody or antibody binding fragment can comprise a light chain variable region comprising at least about 99% sequence identity to any one of SEQ ID NOs: 100-106.

For example, the antibody or antibody binding fragment can comprise a light chain variable region comprising at least about 99.5% sequence identity to any one of SEQ ID NOs: 100-106.

For example, the antibody or antibody binding fragment can comprise a light chain variable region comprising at least about 99.9% sequence identity to any one of SEQ ID NOs: 100-106.

For ease of reference, sequences for SEQ IDs 93-106 are described in Table 3 below. In the table, CDR sequences within each chain are bolded and underlined.

TABLE 3
Illustrative Heavy Chain or Light Chain
Variable Regions for Anti-IBV NA Antibodies

| | Antibody | | SEQ |
|---|---|---|---|
| Chain | of | | ID |
| Type | Origin | AMINO ACID SEQUENCE | NO: |
| Heavy | 1G05 | QVQLQESGPGLVRPSETLSLTCTVSGDSIGGSYWN | 93 |
| Chain | | WIRQPPGKGLQWIGYIYYTGITNYNPSLKSRVTMS | |
| | | LDTSKNQISLKMDSVTAADTALYFCARGDYSGYD | |
| | | RDVQVELMDVWGKGTTVTVSS | |
| | 2E01 | EVQLVQSGAEVKKPGSSVKVSCKASGYTFINHALS | 94 |
| | | WVRQAPGQGLEWVGGIIPIFGLAKYGQKFQDRVT | |
| | | ITADESTKTAYMDLRSLRSDDTAVYYCARDTVAV | |
| | | YEDFDWSSPYFFYMDVWGKGTTVTVSS | |
| Variable | 1A03 | QVQLQESGPGLVKPSQTLSLTCTVSGGSISSGGNY | 95 |
| Region | | WSWIRQHPGKGLEWIGFISYSGSTYFTPSLNSRLTI | |
| | | SVDTTNNHFSLKLSSVTGADTAVYYCARGRGYCS | |
| | | RGATCYNFYMDVWGVGTTVTVSS | |
| | 2D10 | QVQLQESGPGLVKPSETLSLTCTVSGDSISGSSYY | 96 |
| | | WGWIRQPPGKGLEWIGSIYYSGITYYNPSLKSRVTI | |
| | | YVDTSKNQFSLKLNSATAADTAVYYCARLYTKSS | |
| | | NANYWGQGTLVTVSS | |
| | 1D05 | EVQLVESGGSVVQPGRSLRLSCAAFGFSFSAYGM | 97 |
| | | HWVRQAPGKGLEWVTLLGYDGTDQWVAESVKG | |
| | | RFTVSRDNSRNTVILQMDSLRAEDTAVYFCARGA | |
| | | RPYYTDYRDHRPSYFYYHMDVWGAGTTVTVSS | |
| | 2H09 | EVQLVESGGGLVQPGGSLRLSCAASGVTLDNYWV | 98 |
| | | HWVRQVPGKGLVWVSRINADGTSKTYADSVKGR | |
| | | FTISRDTTRNTLFLQMNSLRGDDTALYFCARGGLY | |
| | | SSDAFDVWGQGTMVTVSS | |
| | 3CO1 | EVQLVESGGGLVQPGGSLRLSCAASGFPFSHYYM | 99 |
| | | HWVRRAPGKGLVWVSRIYSDGTSSSYGDSVKGRF | |
| | | TISRDNAKNILYLQMNSLRAEDSATYYCCRGGYY | |
| | | SLDGFDFWGQGTMVTVSS | |
| Light | 1G05 | DIQMTQSPSSLSASVRDKVTFVCRASQTISIFLNWY | 100 |
| Chain | | QHKPGEAPKLLIYAASRLQSGVPSRFSGSGSGTDFT | |
| | | LTISGLQPEDFATYYCQQSYSAPWTFGQGTKVEIK | |
| | 2E01 | EIVLTQSPATLSLFPGERATLSCRASQSAGSKSLAW | 101 |
| | | YQHKVGQPPRLLINGASSRATGIPDRFSGSGSGPDF | |
| | | NLTISRLEPEDFAVYYCQRYGTSLVTFGGGTKVEI | |
| | | K | |
| Variable | 1A03 | QSALTQPASVSGSPGQSITISCTGTSGDIGGYNLVS | 102 |
| Region | | WYQHHPGRVPKLIIYEDSKRPSGFLNRFSGSKSGNT | |
| | | ASLTISGLQAEDVADYYCCSHAGSVVFGGGTRLT | |
| | | VQ | |
| | 2D10 | DIQMTQSPSTLSASVGDRVTITCRASQSISSWLAW | 103 |
| | | YQQKPGKAPKLLIYDASSLESGVPSRFSGSGSGTEF | |
| | | TLTISSLQSDDFAIYYCQQYHSYSGTFGQGTKVEIK | |
| | 1D05 | QSVLTQPPSVSAAPGQKVTISCSGSSSNIGNNYVSW | 104 |
| | | YQQLPGTAPKLLIYDSDKRPSGIPARFSGSKSGASA | |
| | | TLAITGLQTGDEADYYCGTWDNSLNVLVFGGGTK | |
| | | LAVL | |
| | 2H09 | QSALTQPPSASGSPGQSVTISCTGTSWDVGRYNHV | 105 |
| | | SWYQHHPGKAHKLIIYEVNRRPSGVPDRFSGSKSA | |
| | | NTASLTVSGLQAEDEADYYCSSYTGNNVAVFGGG | |
| | | TKLTVL | |
| | 3C01 | DIQMTQSPLSLSVSEGDRVTITCRASQGIGNDLGW | 106 |
| | | YQLKPGKAPKRLIYGASSLQSGVPSRFSGSGSGTEF | |
| | | TLTISSLQPEDFATYFCLQHSSFPYTFGQGTKVEIK | |

As may be envisioned by one of ordinary skill in the art, the various CDR sequences and FR sequences may be combined in various ways to form new antibodies. Specific combinations of the CDR sequences within or exclusive of the full heavy or light chain variable regions of Table 3, are described in more detail below.

The antibody or antigen-binding fragment thereof can comprise an immunoglobulin heavy chain variable region comprising an amino acid sequence having at least 70% identity to any one of SEQ ID NO: 1-21 and 93-99.

The antibody or antigen-binding fragment thereof can comprise an immunoglobulin light chain variable region comprising an amino acid sequence having at least 70% identity to any one of SEQ ID NOs: 22-41 and 100-106.

The antibody or antigen-binding fragment thereof can comprise (a) an immunoglobulin heavy chain variable region comprising a CDR_{H1} having an amino acid sequence comprising any one of SEQ ID NOs: 1-7, a CDR_{H2} having an amino acid sequence comprising any one of SEQ ID NOs: 8-14, a CDR_{H3} having an amino acid sequence comprising any one of SEQ ID NOs: 15-21, or a combination of any thereof; (b) an immunoglobulin light chain variable region comprising a CDR_{L1} having an amino acid sequence comprising any one of SEQ ID NOs: 22-28, a CDR_{L2} having an amino acid sequence comprising any one of SEQ ID NOs: 29-34, a CDR_{L3} having an amino acid sequence comprising any one of SEQ ID NOs: 35-41, or a combination of any thereof; or (c) a combination thereof

For example, the antibody or antigen-binding fragment thereof can comprise (a) an immunoglobulin heavy chain variable region comprising a CDR_{H1} having an amino acid sequence comprising any one of SEQ ID NOs: 1-7, a CDR_{H2} having an amino acid sequence comprising any one of SEQ ID NOs: 8-14, a CDR_{H3} having an amino acid sequence comprising any one of SEQ ID NOs: 15-21, or a combination thereof; and (b) an immunoglobulin light chain variable region comprising a CDR_{L1} having an amino acid sequence comprising any one of SEQ ID NOs: 22-28, a CDR_{L2} having an amino acid sequence comprising any one of SEQ ID NOs: 29-34, a CDR_{L3} having an amino acid sequence comprising any one of SEQ ID NOs: 35-41, or a combination thereof.

In various embodiments, the antibody or antigen-binding fragment comprises an immunoglobulin heavy chain variable region comprises a CDR_{H1} having an amino acid sequence comprising any one of SEQ ID NOs: 1-7, a CDR_{H2} having an amino acid sequence comprising any one of SEQ ID NOs: 8-14, or a CDR_{H3} having an amino acid sequence comprising any one of SEQ ID NOs: 15-21.

In various embodiments, the antibody or antigen-binding fragment comprises an immunoglobulin heavy chain variable region comprising a CDR_{H1} having an amino acid sequence comprising any one of SEQ ID NOs: 1-7.

The antibody or antigen-binding fragment thereof can comprise an immunoglobulin heavy chain variable region comprising a CDR_{H1} having an amino acid sequence comprising SEQ ID NO: 1.

The antibody or antigen-binding fragment thereof can comprise an immunoglobulin heavy chain variable region comprising a CDR_{H1} having an amino acid sequence comprising SEQ ID NO: 2.

The antibody or antigen-binding fragment thereof can comprise an immunoglobulin heavy chain variable region comprising a CDR_{H1} having an amino acid sequence comprising SEQ ID NO: 3.

The antibody or antigen-binding fragment thereof can comprise an immunoglobulin heavy chain variable region comprising a CDR_{H1} having an amino acid sequence comprising SEQ ID NO: 4.

The antibody or antigen-binding fragment thereof can comprise an immunoglobulin heavy chain variable region comprising a CDR_{H1} having an amino acid sequence comprising SEQ ID NO: 5.

The antibody or antigen-binding fragment thereof can comprise an immunoglobulin heavy chain variable region comprising a CDR_{H1} having an amino acid sequence comprising SEQ ID NO: 6.

The antibody or antigen-binding fragment thereof can comprise an immunoglobulin heavy chain variable region comprising a CDR_{H1} having an amino acid sequence comprising SEQ ID NO: 7.

In various embodiments, the antibody or antigen-binding fragment comprises an immunoglobulin heavy chain variable region comprising a CDR_{H2} having an amino acid sequence comprising any one of SEQ ID NOs: 8-14.

The antibody or antigen-binding fragment thereof can comprise an immunoglobulin heavy chain variable region comprising a CDR_{H2} having an amino acid sequence comprising SEQ ID NO: 8.

The antibody or antigen-binding fragment thereof can comprise an immunoglobulin heavy chain variable region comprising a CDR_{H2} having an amino acid sequence comprising SEQ ID NO: 9.

The antibody or antigen-binding fragment thereof can comprise an immunoglobulin heavy chain variable region comprising a CDR_{H2} having an amino acid sequence comprising SEQ ID NO: 10.

The antibody or antigen-binding fragment thereof can comprise an immunoglobulin heavy chain variable region comprising a CDR_{H2} having an amino acid sequence comprising SEQ ID NO: 11.

The antibody or antigen-binding fragment thereof can comprise an immunoglobulin heavy chain variable region comprising a CDR_{H2} having an amino acid sequence comprising SEQ ID NO: 12.

The antibody or antigen-binding fragment thereof can comprise an immunoglobulin heavy chain variable region comprising a CDR_{H2} having an amino acid sequence comprising SEQ ID NO: 13.

The antibody or antigen-binding fragment thereof can comprise an immunoglobulin heavy chain variable region comprising a CDR_{H2} having an amino acid sequence comprising SEQ ID NO: 14.

In various embodiments, the antibody or antigen-binding fragment comprises an immunoglobulin heavy chain variable region comprising a CDR_{H3} having an amino acid sequence comprising any one of SEQ ID NOs: 15-21.

The antibody or antigen-binding fragment thereof can comprise an immunoglobulin heavy chain variable region comprising a CDR_{H3} having an amino acid sequence comprising SEQ ID NO: 15.

The antibody or antigen-binding fragment thereof can comprise an immunoglobulin heavy chain variable region comprising a CDR_{H3} having an amino acid sequence comprising SEQ ID NO: 16.

The antibody or antigen-binding fragment thereof can comprise an immunoglobulin heavy chain variable region comprising a CDR_{H3} having an amino acid sequence comprising SEQ ID NO: 17.

The antibody or antigen-binding fragment thereof can comprise an immunoglobulin heavy chain variable region comprising a CDR_{H3} having an amino acid sequence comprising SEQ ID NO: 18.

The antibody or antigen-binding fragment thereof can comprise an immunoglobulin heavy chain variable region comprising a CDR_{H3} having an amino acid sequence comprising SEQ ID NO: 19.

The antibody or antigen-binding fragment thereof can comprise an immunoglobulin heavy chain variable region comprising a CDR_{H3} having an amino acid sequence comprising SEQ ID NO: 20.

The antibody or antigen-binding fragment thereof can comprise an immunoglobulin heavy chain variable region comprising a CDR_{H3} having an amino acid sequence comprising SEQ ID NO: 21.

In various embodiments, the antibody or antigen-binding fragment can comprise an immunoglobulin heavy chain variable region comprising a CDR_{L1} having an amino acid sequence comprising any one of SEQ ID NOs: 1-7, a CDR_{H2} having an amino acid sequence comprising any one of SEQ ID NOs: 8-14, and a CDR_{H3} having an amino acid sequence comprising any one of SEQ ID NOs: 15-21.

In various embodiments, the antibody or antigen-binding fragment can comprise an immunoglobulin light chain variable region comprising a CDR_{L1} having an amino acid sequence comprising any one of SEQ ID NOs: 22-28, a CDR_{L2} having an amino acid sequence comprising any one of SEQ ID NOs: 29-34, or a CDR_{L3} having an amino acid sequence comprising any one of SEQ ID NOs: 35-41.

For example, the antibody or antigen-binding fragment can comprise an immunoglobulin light chain variable region comprising a CDR_{L1} having an amino acid sequence comprising any one of SEQ ID NOs: 22-28.

The antibody or antigen-binding fragment can comprise an immunoglobulin light chain variable region comprising a CDR_{L1} having an amino acid sequence comprising SEQ ID NO: 22.

The antibody or antigen-binding fragment can comprise an immunoglobulin light chain variable region comprising a CDR_{L1} having an amino acid sequence comprising SEQ ID NO: 23.

The antibody or antigen-binding fragment can comprise an immunoglobulin light chain variable region comprising a CDR_{L1} having an amino acid sequence comprising SEQ ID NO: 24.

The antibody or antigen-binding fragment can comprise an immunoglobulin light chain variable region comprising a CDR_{L1} having an amino acid sequence comprising SEQ ID NO: 25.

The antibody or antigen-binding fragment can comprise an immunoglobulin light chain variable region comprising a CDR_{L1} having an amino acid sequence comprising SEQ ID NO: 26.

The antibody or antigen-binding fragment can comprise an immunoglobulin light chain variable region comprising a CDR_{L1} having an amino acid sequence comprising SEQ ID NO: 27.

The antibody or antigen-binding fragment can comprise an immunoglobulin light chain variable region comprising a CDR_{L1} having an amino acid sequence comprising SEQ ID NO: 28.

In various embodiments, the antibody or antigen-binding fragment can comprise an immunoglobulin light chain variable region comprising a CDR_{L2} having an amino acid sequence comprising any one of SEQ ID NOs: 29-34.

The antibody or antigen-binding fragment can comprise an immunoglobulin light chain variable region comprising a CDR_{L2} having an amino acid sequence comprising SEQ ID NO: 29.

The antibody or antigen-binding fragment can comprise an immunoglobulin light chain variable region comprising a CDR_{L2} having an amino acid sequence comprising SEQ ID NO: 30.

The antibody or antigen-binding fragment can comprise an immunoglobulin light chain variable region comprising a CDR_{L2} having an amino acid sequence comprising SEQ ID NO: 31.

The antibody or antigen-binding fragment can comprise an immunoglobulin light chain variable region comprising a CDR_{L2} having an amino acid sequence comprising SEQ ID NO: 32.

The antibody or antigen-binding fragment can comprise an immunoglobulin light chain variable region comprising a CDR_{L2} having an amino acid sequence comprising SEQ ID NO: 33.

The antibody or antigen-binding fragment can comprise an immunoglobulin light chain variable region comprising a CDR_{L2} having an amino acid sequence comprising SEQ ID NO: 34.

In various embodiments, the antibody or antigen-binding fragment can comprise an immunoglobulin light chain variable region comprising a CDR_{L3} having an amino acid sequence comprising any one of SEQ ID NOs: 35-41.

The antibody or antigen-binding fragment can comprise an immunoglobulin light chain variable region comprising a CDR_{L3} having an amino acid sequence comprising SEQ ID NO: 35.

The antibody or antigen-binding fragment can comprise an immunoglobulin light chain variable region comprising a CDR_{L3} having an amino acid sequence comprising SEQ ID NO: 36.

The antibody or antigen-binding fragment can comprise an immunoglobulin light chain variable region comprising a CDR_{L3} having an amino acid sequence comprising SEQ ID NO: 37.

The antibody or antigen-binding fragment can comprise an immunoglobulin light chain variable region comprising a CDR_{L3} having an amino acid sequence comprising SEQ ID NO: 38.

The antibody or antigen-binding fragment can comprise an immunoglobulin light chain variable region comprising a CDR_{L3} having an amino acid sequence comprising SEQ ID NO: 39.

The antibody or antigen-binding fragment can comprise an immunoglobulin light chain variable region comprising a CDR_{L3} having an amino acid sequence comprising SEQ ID NO: 40.

The antibody or antigen-binding fragment can comprise an immunoglobulin light chain variable region comprising a CDR_{L3} having an amino acid sequence comprising SEQ ID NO: 41.

In various embodiments, the antibody or antigen-binding fragment can comprise an immunoglobulin light chain variable region comprising a CDR_{L1} having an amino acid sequence comprising any one of SEQ ID NOs: 22-28, a CDR_{L2} having an amino acid sequence comprising any one of SEQ ID NOs: 29-34, and a CDR_{L3} having an amino acid sequence comprising any one of SEQ ID NOs: 35-41.

In various embodiments, the antibody or antigen-binding fragment can comprise an immunoglobulin heavy chain variable region comprising a CDR_{H1} having an amino acid sequence comprising any one of SEQ ID NOs: 1-7, a CDR_{H2} having an amino acid sequence comprising any one of SEQ ID NOs: 8-14, and a CDR_{H3} having an amino acid sequence comprising any one of SEQ ID NOs: 15-21; and an immunoglobulin light chain variable region comprising a CDR_{L1} having an amino acid sequence comprising any one of SEQ ID NOs: 22-28, a CDR_{L2} having an amino acid sequence comprising any one of SEQ ID NOs: 29-34, and a CDR_{L3} having an amino acid sequence comprising any one of SEQ ID NOs: 35-41.

In various embodiments, the antibody or antigen-binding fragment can comprise an immunoglobulin heavy chain variable region comprising: (a) a CDR_{L1} having an amino acid sequence comprising SEQ ID NO: 1, a CDR_{H2} having an amino acid sequence comprising SEQ ID NO: 8, and a CDR_{H3} having an amino acid sequence comprising SEQ ID NO: 15; (b) a CDR_{L1} having an amino acid sequence comprising SEQ ID NO: 2, a CDR_{H2} having an amino acid sequence comprising SEQ ID NO: 9, and a CDR_{H3} having an amino acid sequence comprising SEQ ID NO: 16; (c) a CDR_{H1} having an amino acid sequence comprising SEQ ID NO: 3, a CDR_{H2} having an amino acid sequence comprising SEQ ID NO: 10, and a CDR_{H3} having an amino acid sequence comprising SEQ ID NO: 17; (d) a CDR_{H1} having an amino acid sequence comprising SEQ ID NO: 4, a CDR_{H2} having an amino acid sequence comprising SEQ ID NO: 11, and a CDR_{H3} having an amino acid sequence comprising SEQ ID NO: 18; (e) a CDR_{H1} having an amino acid sequence comprising SEQ ID NO: 5, a CDR_{H2} having an amino acid sequence comprising SEQ ID NO: 12, and a CDR_{H3} having an amino acid sequence comprising SEQ ID NO: 19; (f) a CDR_{H1} having an amino acid sequence comprising SEQ ID NO: 6, a CDR_{H2} having an amino acid sequence comprising SEQ ID NO: 13, and a CDR_{H3} having an amino acid sequence comprising SEQ ID NO: 20; or (g) a CDR_{H1} having an amino acid sequence comprising SEQ ID NO: 7, a CDR_{H2} having an amino acid sequence comprising SEQ ID NO: 14, and a CDR_{H3} having an amino acid sequence comprising SEQ ID NO: 21.

In various embodiments, the antibody or antigen-binding fragment can comprise an immunoglobulin light chain variable region comprising: (a) a CDR_{L1} having an amino acid sequence comprising SEQ ID NO: 22, a CDR_{L2} having an amino acid sequence comprising SEQ ID NO: 29, and a CDR_{L3} having an amino acid sequence comprising SEQ ID NO: 35; (b) a CDR_{L1} having an amino acid sequence comprising SEQ ID NO: 23, a CDR_{L2} having an amino acid sequence comprising SEQ ID NO: 30, and a CDR_{L3} having an amino acid sequence comprising SEQ ID NO: 36; (c) a CDR_{L1} having an amino acid sequence comprising SEQ ID NO: 24, a CDR_{L2} having an amino acid sequence comprising SEQ ID NO: 31, and a CDR_{L3} having an amino acid sequence comprising SEQ ID NO: 37; (d) a CDR_{L1} having an amino acid sequence comprising SEQ ID NO: 25, a CDR_{L2} having an amino acid sequence comprising SEQ ID NO: 32, and a CDR_{L3} having an amino acid sequence comprising SEQ ID NO: 38; (e) a CDR_{L1} having an amino acid sequence comprising SEQ ID NO: 26, a CDR_{L2} having an amino acid sequence comprising SEQ ID NO: 33, and a CDR_{L3} having an amino acid sequence comprising SEQ ID NO: 39; (f) a CDR_{L1} having an amino acid sequence comprising SEQ ID NO: 27, a CDR_{L2} having an amino acid sequence comprising SEQ ID NO: 34, and a CDR_{L3} having an amino acid sequence comprising SEQ ID NO: 40; or (g) a CDR_{L1} having an amino acid sequence comprising SEQ ID NO: 28, a CDR_{L2} having an amino acid sequence comprising SEQ ID NO: 30, and a CDR_{L3} having an amino acid sequence comprising SEQ ID NO: 41.

An illustrative antibody of the present invention can comprise (a) an immunoglobulin heavy chain variable region comprising a CDR_{H1} having an amino acid sequence comprising SEQ ID NO: 1, a CDR_{H2} having an amino acid sequence comprising SEQ ID NO: 8, and a CDR_{H3} having an amino acid sequence comprising SEQ ID NO: 15; and (b) an immunoglobulin light chain variable region comprising a CDR_{L1} having an amino acid sequence comprising SEQ ID NO: 22, a CDR_{L2} having an amino acid sequence comprising SEQ ID NO: 29, and a CDR_{L3} having an amino acid sequence comprising SEQ ID NO: 35.

A second illustrative antibody of the present invention can comprise (a) an immunoglobulin heavy chain variable region comprising a CDR_{H1} having an amino acid sequence comprising any one of SEQ ID NO: 2, a CDR_{H2} having an amino acid sequence comprising SEQ ID NO: 9 and a CDR_{H3} having an amino acid sequence comprising SEQ ID NO: 16; and (b) an immunoglobulin light chain variable region comprising a CDR_{L1} having an amino acid sequence comprising SEQ ID NO: 23 a CDR_{L2} having an amino acid sequence comprising SEQ ID NO: 30, and a CDR_{L3} having an amino acid sequence comprising SEQ ID NO: 36.

A third illustrative antibody of the present invention can comprise an (a) an immunoglobulin heavy chain variable region comprising a CDR_{H1} having an amino acid sequence comprising SEQ ID NO: 3, a CDR_{H2} having an amino acid sequence comprising SEQ ID NO: 10, and a CDR_{H3} having an amino acid sequence comprising SEQ ID NO: 17; and (b) an immunoglobulin light chain variable region comprising a CDR_{L1} having an amino acid sequence comprising SEQ ID NO: 24, a CDR_{L2} having an amino acid sequence comprising SEQ ID NO: 31, and a CDR_{L3} having an amino acid sequence comprising SEQ ID NO: 37.

A fourth illustrative antibody of the present invention can comprise an (a) an immunoglobulin heavy chain variable region comprising a CDR_{H1} having an amino acid sequence comprising SEQ ID NO: 4, a CDR_{H2} having an amino acid sequence comprising SEQ ID NO: 11, and a CDR_{H3} having an amino acid sequence comprising SEQ ID NO: 18; and (b) an immunoglobulin light chain variable region comprising a CDR_{L1} having an amino acid sequence comprising SEQ ID NO: 25, a CDR_{L2} having an amino acid sequence comprising SEQ ID NO: 32, and a CDR_{L3} having an amino acid sequence comprising SEQ ID NO: 38.

A fifth illustrative antibody of the present invention can comprise an (a) an immunoglobulin heavy chain variable region comprising a CDR_{H1} having an amino acid sequence comprising SEQ ID NO: 5, a CDR_{H2} having an amino acid sequence comprising SEQ ID NO: 12, and a CDR_{H3} having an amino acid sequence comprising SEQ ID NO: 19; and (b) an immunoglobulin light chain variable region comprising a CDR_{L1} having an amino acid sequence comprising SEQ ID NO: 26, a CDR_{L2} having an amino acid sequence comprising SEQ ID NO: 33, and a CDR_{L3} having an amino acid sequence comprising SEQ ID NO: 39.

A sixth illustrative antibody of the present invention can comprise an (a) an immunoglobulin heavy chain variable region comprising a CDR_{H1} having an amino acid sequence comprising SEQ ID NO: 6, a CDR_{H2} having an amino acid sequence comprising SEQ ID NO: 13, and a CDR_{H3} having an amino acid sequence comprising SEQ ID NO: 20; and (b) an immunoglobulin light chain variable region comprising a CDR_{L1} having an amino acid sequence comprising SEQ ID NO: 27, a CDR_{L2} having an amino acid sequence comprising SEQ ID NO: 34, and a CDR_{L3} having an amino acid sequence comprising SEQ ID NO: 40.

A seventh illustrative antibody of the present invention can comprise an (a) an immunoglobulin heavy chain variable region comprising a CDR_{L1} having an amino acid sequence comprising SEQ ID NO: 7, a CDR_{H2} having an amino acid sequence comprising SEQ ID NO: 14, and a CDR_{H3} having an amino acid sequence comprising SEQ ID NO: 21; and (b) an immunoglobulin light chain variable region comprising a CDR_{L1} having an amino acid sequence comprising SEQ ID NO: 28, a CDR_{L2} having an amino acid sequence comprising SEQ ID NO: 30, and a CDR_{L3} having an amino acid sequence comprising SEQ ID NO: 41.

As noted above, in various embodiments, the antibody or antigen-binding fragment comprises an immunoglobulin heavy chain variable region having at least about 70% sequence identity to SEQ ID NO: 93-99. For example, in various embodiments, the antibody or antigen-binding fragment can comprise an immunoglobulin heavy chain variable region having at least about 75%, at least about 80%, at least about 85%, at least about 90%, at least about 95%, at least about 96%, at least about 97%, at least about 98%, at least about 99%, at least about 99.5% or at least about 99.9% sequence identity to SEQ ID NOs: 93-99.

In any of the embodiments described herein, the immunoglobulin heavy chain variable region can comprise at least one of SEQ ID NOs: 1-21.

In some embodiments, the immunoglobulin heavy chain variable region comprises at least one of SEQ ID NO: 1, 8, or 15. For example, the immunoglobulin heavy chain variable region can comprise SEQ ID NOs: 1, 8, and 15.

In some embodiments, immunoglobulin heavy chain variable region comprises at least one of SEQ ID NOs: 2, 9, or 16. For example, the immunoglobulin heavy chain variable region can comprise SEQ ID NOs: 2, 9, and 16.

In some embodiments, the immunoglobulin heavy chain variable region comprises at least one of SEQ ID NOs: 3, 10, or 17. For example, the immunoglobulin heavy chain variable region can comprise SEQ ID NOs: 3, 10, and 17.

In some embodiments, the immunoglobulin heavy chain variable region comprises at least one of SEQ ID NOs: 4, 11, or 18. For example, the immunoglobulin heavy chain variable region can comprise SEQ ID NOs: 4, 11, and 18.

In some embodiments, the immunoglobulin heavy chain variable region comprises at least one of SEQ ID NOs: 5, 12, or 19. For example, the immunoglobulin heavy chain variable region can comprise SEQ ID NOs: 5, 12 and 19.

In some embodiments, the immunoglobulin heavy chain variable region comprises at least one of SEQ ID NOs: 6, 13, or 20. For example, the immunoglobulin heavy chain variable region can comprise SEQ ID NOs: 6, 13 and 20.

In some embodiments, the immunoglobulin heavy chain variable region comprises at least one of SEQ ID NOs: 7, 14, or 21. For example, the immunoglobulin heavy chain variable region can comprise SEQ ID NOs: 7, 14, or 21.

As noted above, in various embodiments, the antibody or antigen-binding fragment can comprise an immunoglobulin light chain variable region comprising at least about 70% sequence identity to any one of SEQ ID NOs: 22-41. For example, in various embodiments, the antibody or antigen-binding fragment can comprise an immunoglobulin light chain variable region comprising at least about 75%, at least about 80%, at least about 85%, at least about 90%, or at least about 95%, at least about 96%, at least about 97%, at least about 98%, at least about 99%, at least about 99.5%, or at least about 99.9% sequence identity to any one of SEQ ID NOs: 22-41.

In any of the embodiments described herein, the immunoglobulin light chain variable region can comprise at least one of SEQ ID NOs: 22-41.

In some embodiments, the immunoglobulin light chain variable region comprises at least one of SEQ ID NO: 22, 29, or 35. For example, the immunoglobulin light chain variable region comprises SEQ ID NO: 22, 29 and 35.

In some embodiments, the immunoglobulin light chain variable region comprises at least one of SEQ ID NOs: 23, 30, or 36. For example, the immunoglobulin light chain variable region can comprise SEQ ID NOs: 23, 30 and 36.

In some embodiments, the immunoglobulin light chain variable region comprises at least one of SEQ ID NOs: 24, 31, and 37. For example, the immunoglobulin light chain variable region can comprise SEQ ID NOs: 24, 31, and 37.

In some embodiments, the immunoglobulin light chain variable region comprises at least one of SEQ ID NOs: 25, 32, or 38. For example, the immunoglobulin light chain variable region can comprise SEQ ID NOs: 25, 32, and 38.

In some embodiments, the immunoglobulin light chain variable region comprises at least one of SEQ ID NOs: 26, 33, or 39. For example, the immunoglobulin light chain variable region can comprise SEQ ID NOs: 26, 33, and 39.

In some embodiments, the immunoglobulin light chain variable region comprises at least one of SEQ ID NOs: 27, 34, or 40. For example, the immunoglobulin light chain variable region can comprise SEQ ID NOs: 27, 34, and 40.

In some embodiments, the immunoglobulin light chain variable region comprises at least one of SEQ ID NOs: 28, 30, or 41. For example, the immunoglobulin light chain variable region can comprise SEQ ID NOs: 28, 30 and 40.

In some embodiments, the antibody or antigen-binding fragment can comprise an immunoglobulin heavy chain variable region comprising at least about 70%, at least about 75%, at least about 80%, at least about 85%, at least about 90%, or at least about 95%, at least about 96%, at least about 97%, at least about 98%, at least about 99% or at least about 99.5% sequence identity to any one of SEQ ID NOs: 93-99 and an immunoglobulin light chain variable region comprising at least about 70%, at least about 75%, at least about 80%, at least about 85%, at least about 90%, or at least about 95%, at least about 96%, at least about 97%, at least about 98%, at least about 99% or at least about 99.5% sequence identity to any one of SEQ ID NOs: 100-106.

In some embodiments, the antibody or antibody-binding fragment can comprise an immunoglobulin heavy chain variable region comprising any one of SEQ ID NOs: 93-99 and an immunoglobulin light chain variable region comprising any one of SEQ ID NOs: 100-106.

For example, the antibody or antibody-binding fragment can comprise an immunoglobulin heavy chain variable region comprising SEQ ID NO: 93 and an immunoglobulin light chain variable region comprising any one of SEQ ID NOs: 100-106.

For example, the antibody or antibody-binding fragment can comprise an immunoglobulin heavy chain variable region comprising SEQ ID NO: 94 and an immunoglobulin light chain variable region comprising any one of SEQ ID NOs: 100-106.

For example, the antibody or antibody-binding fragment can comprise an immunoglobulin heavy chain variable region comprising SEQ ID NO: 95 and an immunoglobulin light chain variable region comprising any one of SEQ ID NOs: 100-106.

For example, the antibody or antibody-binding fragment can comprise an immunoglobulin heavy chain variable region comprising SEQ ID NO: 96 and an immunoglobulin light chain variable region comprising any one of SEQ ID NOs: 100-106.

For example, the antibody or antibody-binding fragment can comprise an immunoglobulin heavy chain variable region comprising SEQ ID NO: 97 and an immunoglobulin light chain variable region comprising any one of SEQ ID NOs: 100-106.

For example, the antibody or antibody-binding fragment can comprise an immunoglobulin heavy chain variable region comprising SEQ ID NO: 98 and an immunoglobulin light chain variable region comprising any one of SEQ ID NOs: 100-106.

For example, the antibody or antibody-binding fragment can comprise an immunoglobulin heavy chain variable region comprising SEQ ID NO: 99 and an immunoglobulin light chain variable region comprising any one of SEQ ID NOs: 100-106.

For example, the antibody or antibody-binding fragment can comprise an immunoglobulin heavy chain variable region comprising any one of SEQ ID NOs: 93-99 and an immunoglobulin light chain variable region comprising SEQ ID NO: 100.

For example, the antibody or antibody-binding fragment can comprise an immunoglobulin heavy chain variable region comprising any one of SEQ ID NOs: 93-99 and an immunoglobulin light chain variable region comprising SEQ ID NO: 101.

For example, the antibody or antibody-binding fragment can comprise an immunoglobulin heavy chain variable region comprising any one of SEQ ID NOs: 93-99 and an immunoglobulin light chain variable region comprising SEQ ID NO: 102.

For example, the antibody or antibody-binding fragment can comprise an immunoglobulin heavy chain variable region comprising any one of SEQ ID NOs: 93-99 and an immunoglobulin light chain variable region comprising SEQ ID NO: 103.

For example, the antibody or antibody-binding fragment can comprise an immunoglobulin heavy chain variable region comprising any one of SEQ ID NOs: 93-99 and an immunoglobulin light chain variable region comprising SEQ ID NO: 104.

For example, the antibody or antibody-binding fragment can comprise an immunoglobulin heavy chain variable region comprising any one of SEQ ID NOs: 93-99 and an immunoglobulin light chain variable region comprising SEQ ID NO: 105.

For example, the antibody or antibody-binding fragment can comprise an immunoglobulin heavy chain variable region comprising any one of SEQ ID NOs: 93-99 and an immunoglobulin light chain variable region comprising SEQ ID NO: 106.

An illustrative antibody or antibody binding fragment provided herein can comprise an immunoglobulin heavy chain variable region comprising SEQ ID NO: 93 and an immunoglobulin light chain variable region comprising SEQ ID NO: 100.

A second illustrative antibody or antibody binding fragment provided herein can comprise an immunoglobulin heavy chain variable region comprising SEQ ID NO: 94 and an immunoglobulin light chain variable region comprising SEQ ID NO: 101.

A third illustrative antibody or antibody binding fragment provided herein can comprise an immunoglobulin heavy chain variable region comprising SEQ ID NO: 95 and an immunoglobulin light chain variable region comprising SEQ ID NO: 102.

A fourth illustrative antibody or antibody binding fragment provided herein can comprise an immunoglobulin heavy chain variable region comprising SEQ ID NO: 96 and an immunoglobulin light chain variable region comprising SEQ ID NO: 103.

A fifth illustrative antibody or antibody binding fragment provided herein can comprise an immunoglobulin heavy chain variable region comprising SEQ ID NO: 97 and an immunoglobulin light chain variable region comprising SEQ ID NO: 104.

A sixth illustrative antibody or antibody binding fragment provided herein can comprise an immunoglobulin heavy chain variable region comprising SEQ ID NO: 98 and an immunoglobulin light chain variable region comprising SEQ ID NO: 105.

A seventh illustrative antibody or antibody binding fragment provided herein can comprise an immunoglobulin heavy chain variable region comprising SEQ ID NO: 99 and an immunoglobulin light chain variable region comprising SEQ ID NO: 106.

### Additional Sequences Comprising the Inventive Antibodies

The antibodies described herein are preferably monoclonal antibodies. Accordingly, they can be further characterized by the “V”, “J” and “Junction” amino acid sequences translated from the corresponding “V”, “J”, and in the case of the heavy chains, “D” genes that control the expression of a given antibody. To provide context with the sequences above, each immunoglobulin heavy chain is generated from a recombined V-D-J gene and each immunoglobulin light chain is generated from a recombined V-J gene. The region spanning the V and J segments is called the “junction”. It is the region of highest variability and usually comprises the CDR3 region in both the heavy and light chains (e.g., the CDR_{H3} or CDR_{L3}).

Illustrative V, J and Junction sequences obtained from the antibody sequences described above are provided in Tables 4-6 below, along with their SEQ ID NOs.

TABLE 4
Illustrative “V” sequences of antibodies
specific to IBV-NA.

| | | SEQ | |
|---|---|---|---|
| V-Se- | | ID | Anti- |
| quence | AMINO ACID SEQUENCE | NO: | body |
| Heavy | QVQLQESGPGLVRPSETLSLTCTVSGDSIGGSYW | 107 | 1G05 |
| Chain | NWIRQPPGKGLQWIGYIYYTGITNYNPSLKSRVT | | |
| | MSLDTSKNQISLKMDSVTAADTALYFCAR | | |
| | EVQLVQSGAEVKKPGSSVKVSCKASGYTFINHA | 108 | 2E01 |
| | LSWVRQAPGQGLEWVGGIIPIFGLAKYGQKFQD | | |
| | RVTITADESTKTAYMDLRSLRSDDTAVYYCAR | | |
| | QVQLQESGPGLVKPSQTLSLTCTVSGGSISSGGN | 109 | 1A03 |
| | YWSWIRQHPGKGLEWIGFISYSGSTYFTPSLNSR | | |
| | LTISVDTTNNHFSLKLSSVTGADTAVYYCAR | | |
| | QVQLQESGPGLVKPSETLSLTCTVSGDSISGSSY | 110 | 2D10 |
| | YWGWIRQPPGKGLEWIGSIYYSGITYYNPSLKSR | | |
| | VTIYVDTSKNQFSLKLNSATAADTAVYYCAR | | |
| | EVQLVESGGSVVQPGRSLRLSCAAFGFSFSAYG | 111 | 1D05 |
| | MHWVRQAPGKGLEWVTLLGYDGTDQWVAES | | |
| | VKGRFTVSRDNSRNTVILQMDSLRAEDTAVYFC | | |
| | AR | | |
| | EVQLVESGGGLVQPGGSLRLSCAASGVTLDNY | 112 | 2H09 |
| | WVHWVRQVPGKGLVWVSRINADGTSKTYADS | | |
| | VKGRFTISRDTTRNTLFLQMNSLRGDDTALYFC | | |
| | AR | | |
| | EVQLVESGGGLVQPGGSLRLSCAASGFPFSHYY | 113 | 3C01 |
| | MHWVRRAPGKGLVWVSRIYSDGTSSSYGDSVK | | |
| | GRFTISRDNAKNILYLQMNSLRAEDSATYYC | | |
| Light | DIQMTQSPSSLSASVRDKVTFVCRASQTISIFLN | 114 | 1G05 |
| Chain | WYQHKPGEAPKLLIYAASRLQSGVPSRFSGSGS | | |
| | GTDFTLTISGLQPEDFATYYCQQSYSA | | |
| | EIVLTQSPATLSLFPGERATLSCRASQSAGSKSLA | 115 | 2E01 |
| | WYQHKVGQPPRLLINGASSRATGIPDRFSGSGS | | |
| | GPDFNLTISRLEPEDFAVYYCQRYGTS | | |
| | QSALTQPASVSGSPGQSITISCTGTSGDIGGYNLV | 116 | 1A03 |
| | SWYQHHPGRVPKLIIYEDSKRPSGFLNRFSGSKS | | |
| | GNTASLTISGLQAEDVADYYCCSHAGSVV | | |
| | DIQMTQSPSTLSASVGDRVTITCRASQSISSWLA | 117 | 2D10 |
| | WYQQKPGKAPKLLIYDASSLESGVPSRFSGSGS | | |
| | GTEFTLTISSLQSDDFAIYYCQQYHSY | | |
| | QSVLTQPPSVSAAPGQKVTISCSGSSSNIGNNYV | 118 | 1D05 |
| | SWYQQLPGTAPKLLIYDSDKRPSGIPARFSGSKS | | |
| | GASATLAITGLQTGDEADYYCGTWDNSLN | | |
| | QSALTQPPSASGSPGQSVTISCTGTSWDVGRYN | 119 | 2H09 |
| | HVSWYQHHPGKAHKLIIYEVNRRPSGVPDRFSG | | |
| | SKSANTASLTVSGLQAEDEADYYCSSYTGNN | | |
| | DIQMTQSPLSLSVSEGDRVTITCRASQGIGNDLG | 120 | 3C01 |
| | WYQLKPGKAPKRLIYGASSLQSGVPSRFSGSGS | | |
| | GTEFTLTISSLQPEDFATYFCLQHSSF | | |

TABLE 5
Illustrative “Junction” sequences of
antibodies specific to IBV-NA.

| | | SEQ | |
|---|---|---|---|
| | | ID | |
| Junction | AMINO ACID SEQUENCE | NO: | Antibody |
| Heavy | CARGDYSGYDRDVQVEL | 121 | 1G05 |
| Chain | MDVW | | |
| | CARDTVAVYEDFDWSSPY | 122 | 2E01 |
| | DVW | | |
| FFYM | | | |
| | CARGRGYCSRGATCY | 123 | 1A03 |
| | NFYMDVW | | |
| | CARLYTKSSNANYW | 124 | 2D10 |
| | CARGARPYYTDYRDHRP | 125 | 1D05 |
| | SYFYYHMDVW | | |
| | CARGGLYSSDAFDVW | 126 | 2H09 |
| | CCRGGYYSLDGFDFW | 127 | 3C01 |
| Light | CQQSYSAPWTF | 128 | 1G05 |
| Chain | | | |
| | CQRYGTSLVTF | 129 | 2E01 |
| | CCSHAGSVVF | 130 | 1A03 |
| | CQQYHSYSGTF | 131 | 2D10 |
| | CGTWDNSLNVLVF | 132 | 1D05 |
| | CSSYTGNNVAVF | 133 | 2H09 |
| | CLQHSSFPYTF | 134 | 3C01 |

TABLE 6
Illustrative “J” sequences of
antibodies specific to IBV-NA

| | | SEQ | |
|---|---|---|---|
| | | ID | |
| J-sequence | AMINO ACID SEQUENCE | NO: | Antibody |
| Heavy Chain | MDVWGKGTTVTVSS | 135 | 1G05 |
| | YFFYMDVWGKGTTVTVSS | 136 | 2E01 |
| | YMDVWGVGTTVTVSS | 137 | 1A03 |
| | YWGQGTLVTVSS | 138 | 2D10 |
| | YYHMDVWGAGTTVTVSS | 139 | 1D05 |
| | DAFDVWGQGTMVTVSS | 140 | 2H09 |
| | DGFDFWGQGTMVTVSS | 141 | 3C01 |
| Light Chain | WTFGQGTKVEIK | 142 | 1G05 |
| | TFGGGTKVEIK | 143 | 2E01 |
| | GGGTRLTVQ | 144 | 1A03 |
| | TFGQGTKVEIK | 145 | 2D10 |
| | VFGGGTKLAVL | 146 | 1D05 |
| | AVFGGGTKLTVL | 147 | 2H09 |
| | YTFGQGTKVEIK | 148 | 3C01 |

Accordingly, the antibody or antigen-binding fragment described herein can comprise an amino acid sequence comprising at least one of SEQ ID NOs: 107-148. For example, the antibody or antigen-binding fragment can comprise a “V” region having an amino acid sequence comprising at least one of SEQ ID NOs: 107-120. As another example, the antibody or antigen-binding fragment can comprise a Junction region having an amino acid sequence comprising at least one of SEQ ID NOs: 121-134. As another example, the antibody or antigen-binding fragment can comprise a “J” region having an amino acid sequence comprising at least one of SEQ ID NOs: 135-148.

### Derivatives and Synthetically Synthesized Antibodies or Binding Moieties.

Also provided are peptides, polypeptides and/or proteins derived from any of the antibodies or antibody binding fragments described herein. Generally, as used herein, the derivatives provided here are substantially similar to the antibodies or antibody binding fragments described herein. For example, they may contain one or more conservative substitutions in their amino acid sequences or may contain a chemical modification. The derivatives and modified peptides/polypeptides/proteins all are considered “structurally similar” which means they retain the structure (e.g., the secondary, tertiary or quarternary structure) of the parent molecule and are expected to interact with the antigen in the same way as the parent molecule.

A class of synthetically derived antibodies or antigen-binding moieties can be generated by conservatively mutating resides on the parent molecule to generate a peptide, polypeptide or protein maintaining the same activity as the parent molecule. Representative conservative substitutions are known in the art and are also summarized here.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell

A second way to generate a functional peptide/polypeptide or protein based on the sequences provided herein is through the use of computational, “in-silico” design. For example, computationally designed antibodies or antigen-binding fragments may be designed using standard methods of the art. For example, see Strauch E M et al., (Nat Biotechnol. 2017 July; 35(7):667-671), Fleishman S J et al., (Science. 2011 May 13; 332(6031):816-21), and Koday M T et al., (PLoS Pathog. 2016 Feb. 4; 12(2):e1005409), each incorporated by reference in their entirety.

In various embodiments, an antibody or antibody binding fragment thereof is provided that binds an influenza B virus and is structurally similar to any of the antibodies described herein. That is it has the same secondary, tertiary or quaternary structure as the antibodies or antigen-binding fragments described herein. For example, the antibody or antigen-binding fragment can have a tertiary structure that is structurally similar to a single CDR loop. For example, the antibody or antigen-binding fragment can have a tertiary structure that is structurally similar to a CDR_{H3} loop, e.g., a loop comprising SEQ ID NOs: 15-21 or any combination thereof.

In various embodiments, the antibody can comprise at least one amino acid substitution, deletion, or insertion in a variable region, a hinge region or an Fc region t relative to the sequence of a wild-type variable region, hinge region or a wild-type Fc region.

For example, the antibody can comprise an Fc region that contains at least one amino acid substitution, deletion, or insertion relative to the sequence of a wild-type Fc region. In various embodiments, this substitution, deletion or insertion can prevent or reduce recycling of the antibody (e.g., in vivo).

In various embodiments, the antibody or antigen-binding fragment can comprise a heavy chain variable region and/or light chain variable region comprising at least one amino acid substitution, deletion, or insertion as compared to any one of SEQ ID NOs: 1-106.

Further, as described further below, the antibodies or antigen-binding fragments described herein can be expressed recombinantly (e.g., using a recombinant cell line or recombinant organism). Accordingly, the antibodies or antigen-binding fragments may comprise post-translational modifications (e.g., glycosylation profiles, methylation) that differs from naturally occurring antibodies.

### Binding and Function of the Antibodies and Antigen-Binding Fragments

The antibodies and antigen-binding fragments thereof described herein have some measure of binding affinity to an influenza B virus. Most preferably, the antibody or antigen-binding fragment binds to a neuraminidase. In various embodiments, the neuraminidase may be expressed on the surface of the influenza B virus (i.e., is an Influenza B Virus neuraminidase or IBV-NA). Further, the antibodies and antigen-binding fragments herein may have a certain affinity for a specific epitope on the neuraminidase. The epitope may comprise, for example, an active site of an IBV neuraminidase, such as provided herein as SEQ ID NO: 149.

In various embodiments, the antibody or antigen-binding fragment interacts with at least one active site residue of the neuraminidase. For example, the antibody or antigen-binding fragment can interact with at least one active site residue of an IBV-NA. A representative amino acid sequence (SEQ ID NO: 149) of an active site of an IBV neuraminidase (obtained from the IBV strain: B/Phuket/3073/2013) is shown in Table 7 below.

TABLE 7
Amino Acid Sequence of an IBV neuraminidase

| | |
|---|---|
| Influenza Virus | WTYPRLSCPGSTFQKALLIS |
| Neuraminidase | PHRFGETKGNSAPLIIREPF |
| B/Phuket/ | IACGPKECKHFALTHYAAQP |
| 3073/2013 | GGYYNGTREDRNKLRHLISV |
| SEQ ID | KLGKIPTVENSIFHMAAWSG |
| NO: 149 | SACHDGREWTYIGVDGPDSN |
| | ALLKIKYGEAYTDTYHSYAK |
| | NILRTQESACNCIGGDCYLM |
| | ITDGPASGISECRFLKIREG |
| | RIIKEIFPTGRVKHTEECTC |
| | GFASNKTIECACRDNSYTAK |
| | RPFVKLNVETDTAEIRLMCT |
| | KTYLDTPRPNDGSITGPCES |
| | DGDEGSGGIKGGFVHQRMAS |
| | KIGRWYSRTMSKTKRMGMGL |
| | YVKYDGDPWTDSEALALSGV |
| | MVSMEEPGWYSFGFEIKDKK |
| | CDVPCIGIEMVHDGGKTTWH |
| | SAATAIYCLMG SGQLLWDT |
| | VTGVNMTL |

In various embodiments, the antibody or antibody binding fragment described herein can interact with one or more residue selected from the group consisting of: R147, K435, R116, R292, R374, Y409, D149, and E226 according to the amino acid numbering of SEQ ID NO: 149. Preferably, when the antibody or antibody-binding fragment interacts with one or more of these residues, it comprises a CDR_{H3} region comprising SEQ ID NO: 15.

In various embodiments, the antibody or antibody binding fragment described herein can interact with one or more residue selected from the group consisting of: H134, R147, R116, and R374 according to the amino acid numbering of SEQ ID NO: 149. Preferably, when the antibody or antibody-binding fragment interacts with one or more of these residues, it comprises a CDR_{H3} region comprising SEQ ID NO: 16.

Therefore, an antibody or antigen-binding fragment having specific affinity for an IBV neuraminidase is provided, wherein the antibody or antigen-binding fragment binds to an active site residue of the neuraminidase, wherein the active site residue comprises at least one residue selected from R147, K435, R116, R292, R374, Y409, D149, E226 H134, R147, R116, and R374, according to the amino acid numbering of SEQ ID NO: 149.

The binding of the antibody or antigen-binding fragment can neutralize or inhibit the ability of the neuraminidase to do its normal function which is to cleave sialic acid receptors to facilitate the release of viral particles from infected cells. In various embodiments, the antibodies and/or binding fragment inhibit the function of the neuraminidase to cleave its substrate with an IC50 of about 0.0001 μg/ml to about 30 μg/ml. For example, the antibody or antigen-binding fragment can have an IC50 of about 0.001 μg/ml to about 30 μg/ml. The inhibitory function of the antibody or antigen-binding fragment can be determined by measuring, for example, the ability of the neuraminidase to cleave its substrate (sialic acid) in the presence or absence of the antibody or antigen-binding fragment.

### Humanized, Monoclonal and IgG Antibodies

In various embodiments, the antibody or antigen-binding fragment described herein is humanized. “Humanized” antibodies are generally chimeric or mutant monoclonal antibodies from mouse, rat, hamster, rabbit or other species, bearing human constant and/ir variable region domains or specific changes. Techniques for generating a so-called “humanized” antibody are well known to those of skill in the art.

In various embodiments, the antibody or antigen-binding fragment described herein is a monoclonal antibody. As used herein, the term “monoclonal antibodies” refer to antibodies or antigen-binding fragments that are expressed from the same genetic sequence or sequences and consist of identical antibody molecules.

In various embodiments, the antibody or antigen-binding fragment described herein is an IgG type antibody. For example, the antibody or antigen-binding fragment can be an IgG1, IgG2, IgG3, or an IgG4 type antibody.

### Antibody Production

Methods for producing antibodies of the invention are known in the art. For example, DNA molecules encoding light chain variable regions and/or heavy chain variable regions can be chemically synthesized. Synthetic DNA molecules can be ligated to other appropriate nucleotide sequences, including, e.g., constant region coding sequences, and expression control sequences, to produce conventional gene expression constructs encoding the desired antibody. Production of defined gene constructs is within routine skill in the art.

Nucleic acids encoding desired antibodies can be incorporated (ligated) into expression vectors, which can be introduced into host cells through conventional transfection or transformation techniques. Illustrative host cells are E. coli cells, Chinese hamster ovary (CHO) cells, HeLa cells, baby hamster kidney (BHK) cells, monkey kidney cells (COS), human hepatocellular carcinoma cells (e.g., Hep G2), human embryonal kidney (HEK) cells and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. If the engineered gene is to be expressed in eukaryotic host cells, e.g., CHO cells, it is first inserted into an expression vector containing a suitable eukaryotic promoter, a secretion signal, a poly A sequence, and a stop codon, and, optionally, may contain enhancers, and various introns. This expression vector optionally contains sequences encoding all or part of a constant region, enabling an entire, or a part of, a heavy or light chain to be expressed. The gene construct can be introduced into eukaryotic host cells using conventional techniques. The host cells express VL or VH fragments, VL-VH heterodimers, VH-VL or VL-VH single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e.g., a heavy chain variable region) or a light chain (e.g., a light chain variable region). In other embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In still other embodiments, a host cell is co-transfected with more than one expression vector (e.g., one expression vector encoding a polypeptide comprising an entire, or part of, a heavy chain or heavy chain variable region, and another expression vector encoding a polypeptide comprising an entire, or part of, a light chain or light chain variable region).

A polypeptide comprising an immunoglobulin heavy chain variable region or light chain variable region can be produced by growing (culturing) a host cell transfected with an expression vector encoding such variable region, under conditions that permit expression of the polypeptide. Following expression, the polypeptide can be harvested and purified or isolated using techniques known in the art, e.g., using affinity tags such as glutathione-S-transferase (GST) and histidine tags.

A monoclonal antibody, or an antigen-binding fragment of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The intact antibody (or antigen-binding fragment of the antibody) can be harvested and purified or isolated using techniques known in the art, e.g., Protein A, Protein G, affinity tags such as glutathione-S-transferase (GST) and histidine tags. It is within ordinary skill in the art to express the heavy chain and the light chain from a single expression vector or from two separate expression vectors.

Therefore, in various embodiments, a nucleic acid is provided, the nucleic acid comprising a nucleotide sequence encoding the antibody or antigen-binding fragment described herein. The skilled man will appreciate that functional variants of these nucleic acid molecules are also intended to be a part of the present invention. Functional variants are nucleic acid sequences that can be directly translated, using the standard genetic code, to provide an amino acid sequence identical to that translated from the parental nucleic acid molecules.

The nucleic acid can comprise, for example, a nucleotide sequence comprising any one of SEQ ID NOs 150-163 as described in the Table 8 below.

TABLE 8
Illustrative Nucleic Acid Sequences Encoding
Portions of Inventive Antibodies

| | | SEQ |
|---|---|---|
| | | ID |
| Name | NUCLEIC ACID SEQUENCE | NO: |
| 1G05H | CAGGTGCAGCTGCAGGAGTCGGGCCCAGGACTGG | 150 |
| Homo sapiens | TGAGGCCTTCGGAGACCCTGTCCCTCACCTGCACT | |
| [clone = 1G05H] | GTCTCTGGTGACTCCATCGGTGGTAGCTACTGGAA | |
| Human heavy chain | CTGGATCCGGCAGCCCCCAGGGAAGGGACTGCAG | |
| clone 1G01 | TGGATTGGCTACATTTACTATACTGGGATCACCAA | |
| immunoglobulin variable | CTACAACCCCTCCCTCAAGAGCCGAGTCACCATGT | |
| region, mRNA, partial | CACTCGACACGTCCAAGAACCAGATCTCCCTGAA | |
| CDS | AATGGACTCTGTGACCGCTGCGGACACGGCCCTTT | |
| | ATTTCTGTGCGAGAGGTGACTATAGTGGCTACGAT | |
| | CGGGATGTGCAAGTGGAACTCATGGACGTCTGGG | |
| | GCAAAGGGACCACGGTCACCGTCTCCTCA | |
| 2EO1H | GAGGTGCAGCTGGTGCAATCTGGGGCTGAGGTGA | 151 |
| Homo sapiens | AGAAGCCTGGGTCCTCGGTGAAGGTCTCCTGCAA | |
| [clone-2E01H] | GGCTTCTGGATACACCTTCATCAATCATGCTCTCA | |
| Human heavy chain | GCTGGGTGCGACAGGCCCCTGGGCAAGGGCTTGA | |
| clone 2E01 | GTGGGTGGGAGGGATCATCCCTATCTTTGGTCTGG | |
| immunoglobulin variable | CGAAGTATGGACAAAAGTTCCAGGACAGAGTCAC | |
| region, mRNA, partial | GATTACCGCGGACGAATCCACGAAGACAGCCTAC | |
| CDS | ATGGACCTGAGAAGCCTGAGATCTGACGACACGG | |
| | CCGTTTATTATTGTGCGAGAGACACTGTTGCGGTA | |
| | TACGAGGATTTTGACTGGTCATCACCATACTTCTT | |
| | CTACATGGACGTCTGGGGCAAAGGGACCACGGTC | |
| | ACCGTCTCCTCA | |
| 1A03H | CAGGTGCAGCTGCAGGAGTCGGGTCCAGGACTGG | 152 |
| Homo sapiens | TGAAGCCTTCACAGACCCTGTCCCTCACCTGCACT | |
| [clone = 1A03H] | GTCTCTGGTGGCTCCATCAGCAGTGGTGGTAACTA | |
| Human heavy chain | CTGGAGCTGGATCCGTCAACACCCAGGGAAGGGC | |
| clone 1A03 | CTGGAGTGGATTGGGTTCATCTCTTACAGTGGGAG | |
| immunoglobulin variable | TACCTACTTCACTCCGTCCCTCAACAGCCGACTGA | |
| region, mRNA, partial | CCATATCAGTAGACACGACTAACAACCACTTCTCC | |
| CDS | CTGAAGCTGAGCTCTGTGACTGGCGCGGACACGG | |
| | CCGTTTATTACTGTGCGAGAGGACGGGGATATTGT | |
| | AGTAGAGGTGCCACGTGCTACAATTTCTACATGG | |
| | ACGTCTGGGGCGTAGGGACCACGGTCACCGTCTC | |
| | CTCA | |
| 2D10H | CAGGTGCAGCTGCAGGAGTCGGGCCCAGGACTGG | 153 |
| Homo sapiens | TGAAGCCTTCGGAGACCCTGTCCCTCACCTGCACT | |
| [clone = 2D10H] | GTCTCTGGCGACTCCATCAGCGGTAGTAGTTATTA | |
| Human heavy chain | CTGGGGCTGGATCCGCCAGCCCCCAGGGAAGGGG | |
| clone 2D 10 | CTGGAGTGGATTGGGAGTATCTATTATAGTGGGAT | |
| immunoglobulin variable | CACCTACTACAACCCGTCCCTCAAGAGTCGAGTCA | |
| region, mRNA, partial | CCATATACGTTGACACGTCCAAGAACCAGTTCTCC | |
| CDS | CTGAAGCTGAACTCTGCGACCGCCGCAGACACGG | |
| | CTGTGTATTATTGTGCGAGACTATATACCAAGAGC | |
| | TCAAACGCCAACTACTGGGGCCAGGGAACCCTGG | |
| | TCACCGTCTCCTCA | |
| 1D05H | GAAGTGCAGCTGGTGGAGTCTGGGGGAAGCGTGG | 154 |
| Homo sapiens | TCCAGCCTGGGAGGTCCCTGAGACTCTCCTGTGCA | |
| [clone = 1D05H] | GCCTTTGGATTCAGCTTCAGTGCATATGGCATGCA | |
| Human heavy chain | CTGGGTCCGCCAGGCTCCAGGCAAGGGGCTGGAG | |
| clone 1D05 | TGGGTGACACTTCTAGGGTATGATGGGACTGATC | |
| immunoglobulin variable | AATGGGTTGCAGAGTCCGTGAAGGGCCGATTCAC | |
| region, mRNA, partial | CGTCTCCAGAGACAACTCCAGGAACACAGTAATT | |
| CDS | CTGCAAATGGACAGCCTGAGAGCCGAGGACACGG | |
| | CTGTTTATTTCTGTGCGAGAGGAGCGCGCCCCTAC | |
| | TACACTGACTACAGGGATCACCGACCCTCCTACTT | |
| | CTACTATCACATGGACGTCTGGGGCGCTGGGACC | |
| | ACGGTCACCGTCTCCTCA | |
| 2H09H | GAGGTGCAGCTGGTGGAGTCCGGGGGAGGCTTAG | 155 |
| Homo sapiens | TTCAGCCGGGGGGGTCCCTGAGACTCTCCTGTGCA | |
| [clone = 2H09H] | GCCTCTGGAGTCACCCTCGATAACTATTGGGTACA | |
| Human heavy chain | CTGGGTCCGCCAAGTTCCAGGGAAGGGGCTGGTG | |
| clone 2H09 | TGGGTCTCACGCATTAATGCTGATGGGACCAGTA | |
| immunoglobulin variable | AAACATACGCGGACTCCGTGAAGGGCCGATTCAC | |
| region, mRNA, partial | CATCTCCAGAGACACCACCAGGAACACTCTGTTTC | |
| CDS | TACAAATGAACAGTCTGAGAGGCGACGACACGGC | |
| | TCTGTATTTTTGTGCGAGAGGCGGGCTGTACAGTA | |
| | GTGATGCCTTTGATGTTTGGGGCCAAGGGACAAT | |
| | GGTCACCGTCTCTTCAG | |
| 3C01H | GAGGTGCAGCTGGTGGAGTCCGGGGGAGGCTTAG | 156 |
| Homo sapiens | TTCAGCCTGGGGGGTCCCTGAGACTCTCCTGTGCA | |
| [clone = 3C01H] | GCCTCTGGATTCCCCTTCAGTCACTACTACATGCA | |
| Human heavy chain | CTGGGTCCGCCGAGCTCCAGGGAAGGGGCTGGTT | |
| clone 3CO1 | TGGGTCTCACGTATTTACAGTGATGGGACTAGTTC | |
| immunoglobulin variable | CAGTTACGGGGACTCCGTGAAGGGCCGATTCACC | |
| region, mRNA, partial | ATCTCCAGAGACAACGCCAAGAACATTCTGTATCT | |
| CDS | GCAAATGAACAGTCTGAGAGCCGAAGACTCGGCT | |
| | ACTTACTACTGTTGCCGGGGTGGTTATTATTCTTT | |
| | GGATGGTTTTGATTTCTGGGGCCAAGGGACAATG | |
| | GTCACCGTCTCTTCAG | |
| 1G05L | GACATCCAGATGACCCAGTCTCCATCCTCCCTGTC | 157 |
| Homo sapiens | TGCATCTGTGCGAGACAAAGTCACCTTCGTTTGCC | |
| [clone = 1G05L] | GGGCAAGTCAGACCATAAGCATCTTTTTAAATTGG | |
| Human light chain clone | TATCAACACAAACCAGGGGAAGCCCCCAAGCTCC | |
| 1G05 immunoglobulin | TGATCTATGCTGCGTCCAGGTTGCAAAGTGGGGTC | |
| variable region, mRNA, | CCATCAAGGTTCAGTGGCAGTGGGTCTGGGACAG | |
| partial CDS | ATTTCACTCTCACCATCAGCGGTCTGCAGCCTGAG | |
| | GATTTTGCAACTTACTACTGTCAACAGAGTTACAG | |
| | TGCCCCGTGGACGTTCGGCCAAGGGACCAAGGTG | |
| | GAAATCAAAC | |
| 2E01L | GAAATTGTGTTGACGCAGTCTCCAGCCACCCTGTC | 158 |
| Homo sapiens | TCTGTTTCCAGGGGAGAGAGCCACCCTCTCATGCA | |
| [clone = 2E01L] | GGGCCAGTCAGAGTGCTGGCAGCAAGTCCTTAGC | |
| Human light chain clone | CTGGTACCAGCACAAAGTTGGCCAGCCTCCCAGG | |
| 2E01 immunoglobulin | CTCCTCATCAATGGTGCCTCCAGCAGGGCCACTGG | |
| variable region, mRNA, | CATCCCAGACAGGTTCAGTGGCAGCGGGTCTGGG | |
| partial CDS | CCAGACTTCAATCTAACCATCAGCAGACTGGAGC | |
| | CTGAAGATTTTGCAGTGTATTACTGTCAGCGATAT | |
| | GGTACCTCACTTGTCACCTTCGGCGGCGGGACCAA | |
| | GGTGGAAATCAAAC | |
| 1AO3L | CAGTCTGCCCTGACTCAGCCTGCCTCCGTGTCTGG | 159 |
| Homo sapiens | GTCTCCTGGACAGTCGATCACCATCTCCTGCACTG | |
| [clone = 1A03L] | GAACCAGCGGTGATATTGGGGGTTATAACCTTGTC | |
| Human light chain clone | TCCTGGTACCAACACCACCCAGGCAGAGTCCCCA | |
| 1A03 immunoglobulin | AACTCATAATTTATGAGGACAGTAAACGGCCCTC | |
| variable region, mRNA, | AGGGTTTTTAAATCGCTTCTCTGGCTCCAAGTCTG | |
| partial CDS | GCAACACGGCCTCCCTGACAATCTCTGGGCTCCAG | |
| | GCTGAGGACGTGGCAGATTATTACTGCTGCTCACA | |
| | TGCAGGTAGTGTGGTCTTCGGCGGAGGGACCAGG | |
| | CTGACCGTCCAAG | |
| 2D10L | GACATCCAGATGACCCAGTCTCCTTCCACCCTGTC | 160 |
| Homo sapiens | TGCATCTGTAGGAGACAGAGTCACCATCACTTGCC | |
| [clone = 2D10L] | GGGCCAGTCAGAGTATTAGTAGTTGGTTGGCCTG | |
| Human light chain clone | GTATCAGCAGAAACCAGGGAAAGCCCCTAAACTC | |
| 2D 10 immunoglobulin | CTGATCTATGACGCCTCCAGTTTGGAAAGTGGGGT | |
| variable region, mRNA, | CCCATCAAGGTTCAGCGGCAGTGGATCTGGGACA | |
| partial CDS | GAATTCACTCTCACCATCAGCAGCCTGCAGTCTGA | |
| | TGATTTCGCAATTTATTACTGCCAACAGTATCATA | |
| | GTTATTCAGGGACGTTCGGCCAAGGGACCAAGGT | |
| | GGAAATCAAAC | |
| 1D05L | CAGTCTGTGCTGACGCAGCCGCCCTCAGTGTCTGC | 161 |
| Homo sapiens | GGCCCCAGGACAGAAGGTCACCATCTCCTGCTCT | |
| [clone = 1D05L] | GGAAGCAGCTCCAACATTGGTAATAATTATGTATC | |
| Human light chain clone | CTGGTACCAACAACTCCCAGGAACAGCCCCCAAA | |
| 1D05 immunoglobulin | CTCCTCATTTATGACAGTGATAAGCGACCCTCAGG | |
| variable region, mRNA, | GATTCCTGCCCGATTCTCTGGCTCCAAGTCTGGCG | |
| partial CDS | CGTCAGCCACCCTGGCCATCACCGGACTCCAGACT | |
| | GGGGACGAGGCCGATTATTACTGCGGAACATGGG | |
| | ATAATAGCCTGAATGTTCTAGTATTCGGCGGAGG | |
| | GACCAAGCTGGCCGTCCTAG | |
| 2H09L | CAGTCTGCCCTGACTCAGCCTCCCTCCGCGTCCGG | 162 |
| Homo sapiens | GTCTCCTGGACAGTCAGTCACCATCTCCTGCACTG | |
| [clone = 2H09L] | GAACGAGTTGGGACGTTGGTCGTTATAACCATGTC | |
| Human light chain clone | TCCTGGTACCAACACCACCCAGGCAAAGCCCACA | |
| 2HO9 immunoglobulin | AACTCATCATTTATGAGGTCAATAGGCGGCCCTCA | |
| variable region, mRNA, | GGGGTCCCTGATCGCTTCTCTGGCTCCAAGTCTGC | |
| partial CDS | CAACACGGCCTCCCTGACCGTCTCTGGGCTCCAGG | |
| | CTGAGGATGAGGCTGATTATTACTGCAGCTCATAC | |
| | ACAGGCAACAACGTTGCCGTCTTCGGCGGAGGGA | |
| | CCAAGCTGACCGTCCTGC | |
| 3C01L | GACATCCAGATGACCCAGTCTCCACTCTCCCTGTC | 163 |
| Homo sapiens | TGTATCTGAAGGAGACAGAGTCACCATCACTTGC | |
| [clone = 3C01L] | CGGGCAAGTCAGGGCATTGGAAATGATTTAGGCT | |
| Human light chain clone | GGTATCAATTGAAACCAGGGAAAGCCCCTAAGCG | |
| 3C01 immunoglobulin | CCTGATCTATGGTGCATCCAGTTTGCAAAGTGGGG | |
| variable region, mRNA, | TCCCATCAAGGTTCAGCGGCTCTGGATCTGGGACA | |
| partial CDS | GAATTCACTCTCACAATCAGCAGCCTGCAGCCTGA | |
| | AGATTTTGCAACTTACTTCTGTCTACAGCATAGTA | |
| | GTTTCCCGTATACTTTTGGCCAGGGGACCAAGGTG | |
| | GAAATCAAAC | |

In various embodiments, the nucleic acid comprises a nucleotide sequence encoding an immunoglobulin heavy chain variable region of the antibody or antigen-binding fragment described herein. In various embodiments, the nucleic acid comprises a nucleotide sequence encoding an immunoglobulin light chain variable region of the antibody or antigen-binding fragment described herein. In some embodiments, the nucleic acids encode one or more complementary determining regions (CDR) having the amino acid sequences described herein. As described above, a single nucleic acid may be provided that encodes more than one protein product (e.g., the immunoglobulin light chain and the immunoglobulin heavy chain). Alternatively, two or more separate nucleic acids may be provided each encoding one component of the antibody and/or antigen-binding fragment (e.g., the light chain or the heavy chain).

In various embodiments, an expression vector is provided comprising one or more of the nucleic acids described herein. Vectors can be derived from plasmids such as: F, F1, RP1, Col, pBR322, TOL, Ti, etc; cosmids; phages such as lambda, lambdoid, M13, Mu, P1, P22, Qβ, T-even, T-odd, T2, T4, T7 etc; or plant viruses. Vectors can be used for cloning and/or expression of the binding molecules of the invention and might even be used for gene therapy purposes. Vectors comprising one or more nucleic acid molecules according to the invention operably linked to one or more expression-regulating nucleic acid molecules are also covered by the present invention. The choice of the vector is dependent on the recombinant procedures followed and the host used. Introduction of vectors in host cells can be affected by inter alia calcium phosphate transfection, virus infection, DEAE-dextran mediated transfection, lipofectamin transfection or electroporation. Vectors may be autonomously replicating or may replicate together with the chromosome into which they have been integrated. Preferably, the vectors contain one or more selection markers. The choice of the markers may depend on the host cells of choice. They include, but are not limited to, kanamycin, neomycin, puromycin, hygromycin, zeocin, thymidine kinase gene from Herpes simplex virus (HSV-TK), dihydrofolate reductase gene from mouse (dhfr). Vectors comprising one or more nucleic acid molecules encoding the human binding molecules as described above operably linked to one or more nucleic acid molecules encoding proteins or peptides that can be used to isolate the human binding molecules are also covered by the invention. These proteins or peptides include, but are not limited to, glutathione-S-transferase, maltose binding protein, metal-binding polyhistidine, green fluorescent protein, luciferase and beta-galactosidase.

The expression vector may be transfected into a host cell to induce the translation and expression of the nucleic acid into the heavy chain variable region and/or the light chain variable region. Therefore, a host cell is provided comprising any expression vector described herein. Host cells include, but are not limited to, cells of mammalian, plant, insect, fungal or bacterial origin. Bacterial cells include, but are not limited to, cells from Gram-positive bacteria or Gram-negative bacteria such as several species of the genera Escherichia, such as E. coli, and Pseudomonas. In the group of fungal cells preferably yeast cells are used. Expression in yeast can be achieved by using yeast strains such as inter alia Pichia pastoris, Saccharomyces cerevisiae and Hansenula polymorpha. Furthermore, insect cells such as cells from Drosophila and Sf9 can be used as host cells. Besides that, the host cells can be plant cells such as inter alia cells from crop plants such as forestry plants, or cells from plants providing food and raw materials such as cereal plants, or medicinal plants, or cells from ornamentals, or cells from flower bulb crops. Transformed (transgenic) plants or plant cells are produced by known methods, for example, Agrobacterium-mediated gene transfer, transformation of leaf discs, protoplast transformation by polyethylene glycol-induced DNA transfer, electroporation, sonication, microinjection or bolistic gene transfer. Additionally, a suitable expression system can be a baculovirus system. Expression systems using mammalian cells, such as Chinese Hamster Ovary (CHO) cells, COS cells, BHK cells, NSO cells or Bowes melanoma cells are preferred in the present invention. Since the present invention deals with molecules that may have to be administered to humans, a completely human expression system would be particularly preferred. Therefore, even more preferably, the host cells are human cells. Examples of human cells are, inter alia, HeLa, 911, AT1080, A549, HEK293, 293F and HEK293T cells.

Accordingly, the antibody or antigen-binding fragment can be expressed using a recombinant cell line or recombinant organism.

Further a method is provided for producing an antibody or antigen-binding fragment that binds an influenza B virus, the method comprising growing a host cell as described herein under conditions so that the host cell expresses a polypeptide or polypeptides comprising the immunoglobulin heavy chain variable region and the immunoglobulin light chain variable region, thereby producing the antibody or antigen-binding fragment and purifying the antibody or antigen-binding fragment.

### Pharmaceutical Compositions

Also provided are pharmaceutical compositions comprising at least one antibody or antigen-binding fragment described herein.

Pharmaceutical compositions containing one or more of the antibodies or antigen-binding fragments described herein can be formulated in any conventional manner. Proper formulation is dependent in part upon the route of administration selected. Routes of administration include, but are not limited to parenteral (e.g., intravenous, intra-arterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration. Preferably, the composition is administered parenterally or is inhaled (e.g., intranasal).

The pharmaceutical compositions can also be formulated for parenteral administration, e.g., formulated for injection via intravenous, intra-arterial, subcutaneous, rectal, subcutaneous, intramuscular, intraorbital, intracapsular, intraspinal, intraperitoneal, or intrasternal routes. Dosage forms suitable for parenteral administration include solutions, suspensions, dispersions, emulsions or any other dosage form that can be administered parenterally.

The pharmaceutical composition can be formulated without blood, plasma or a major component of blood or plasma (e.g., blood cells, fibrin, hemoglobin, albumin, etc.).

The pharmaceutical composition can comprise from about 0.001 to about 99.99 wt. % of the antibody or antigen-binding fragment according to the total weight of the composition. For example, the pharmaceutical composition can comprise from about 0.001 to about 1%, about 0.001 to about 5%, about 0.001 to about 10%, about 0.001 to about 15%, about 0.001 to about 20%, about 0.001 to about 25%, about 0.001 to about 30%, about 1 to about 10%, about 1 to about 20%, about 1 to about 30%, about 10 to about 20%, about 10 to about 30%, about 10 to about 40%, about 10 to about 50%, about 20 to about 30%, about 20 to about 40%, about 20 to about 50%, about 20 to about 60%, about 20 to about 70%, about 20 to about 80%, about 20 to about 90%, about 30 to about 40%, about 30 to about 50%, about 30 to about 60%, about 30 to about 70%, about 30 to about 80%, about 30 to about 90%, about 40 to about 50%, about 40 to about 60%, about 40 to about 70%, about 40 to about 80%, about 40 to about 90%, about 50 to about 99.99%, about 50 to about 99%, about 60 to about 99%, about 70 to about 99%, about 80 to about 99%, about 90 to about 99%, about 50 to about 95%, about 60 to about 95%, about 70 to about 95%, about 80 to about 95%, about 90 to about 95%, about 50 to about 90%, about 60 to about 90%, about 70 to about 90%, about 80 to about 90%, about 85 to about 90%, about 50 to about 80%, about 60 to about 80%, about 70 to about 80%, about 75 to about 80%, about 50 to about 70%, about 60 to about 70%, or from about 50 to about 60% of the antibody or antigen-binding fragment by weight according to the total weight of the composition.

The compositions described herein can also comprise one or more pharmaceutically acceptable excipients and/or carriers. The pharmaceutically acceptable excipients and/or carriers for use in the compositions of the present invention can be selected based upon a number of factors including the particular compound used, and its concentration, stability and intended bioavailability; the subject, its age, size and general condition; and the route of administration.

Some examples of materials which can serve as pharmaceutically acceptable carriers in the compositions described herein are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil; and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring, and perfuming agents, preservatives and antioxidants can also be present in the composition, according to the judgment of the formulator based on the desired route of administration.

Pharmaceutically acceptable excipients are identified, for example, in The Handbook of Pharmaceutical Excipients, (American Pharmaceutical Association, Washington, D.C., and The Pharmaceutical Society of Great Britain, London, England, 1968). Additional excipients can be included in the pharmaceutical compositions of the invention for a variety of purposes. These excipients can impart properties which enhance retention of the compound at the site of administration, protect the stability of the composition, control the pH, facilitate processing of the compound into pharmaceutical compositions, and so on. Other excipients include, for example, fillers or diluents, surface active, wetting or emulsifying agents, preservatives, agents for adjusting pH or buffering agents, thickeners, colorants, dyes, flow aids, non-volatile silicones, adhesives, bulking agents, flavorings, sweeteners, adsorbents, binders, disintegrating agents, lubricants, coating agents, and antioxidants.

In various embodiments, the pharmaceutical composition according to the invention can comprise at least one additional antibody or antigen-binding fragment targeting the influenza virus. In these embodiments, the pharmaceutical composition comprises a combination or a mixture of antibodies. The additional antibodies or antigen-binding fragments thereof may be selective for a hemagglutinin (HA) protein or different immunogenic structure present on the influenza virus (such as M2). The additional antibodies or antigen-binding fragments thereof may selectively bind the head or the stalk of the hemagglutinin protein. The additional antibodies or antigen-binding fragments thereof may also be selective for Influenza A viruses, including those selective for Influenza A hemagglutinin and/or neuraminidase proteins.

In some embodiments, the composition further comprises at least one other therapeutic, prophylactic and/or diagnostic agent. Preferably, the therapeutic and/or prophylactic agents are capable of preventing and/or treating an influenza virus infection and/or a condition resulting from such an infection. Therapeutic and/or prophylactic agents include, but are not limited to, anti-viral agents. Such agents can be binding molecules, small molecules, organic or inorganic compounds, enzymes, polynucleotide sequences, anti-viral peptides, etc. The therapeutic and/or prophylactic agent can comprise an M2 inhibitor (e.g., amantadine, rimantadine) and/or a neuraminidase inhibitor (e.g., zanamivir, oseltamivir). In various embodiments, the anti-viral agent can comprise baloxavir, oseltamivir, zanamivir, peramivir, remdesivir or any combination thereof.

The additional antibodies or therapeutic/prophylactic and/or diagnostic agents may be used in combination with the antibodies and antigen-binding fragments of the present invention. “In combination” herein, means simultaneously, as separate formulations, or as one single combined formulation or according to a sequential administration regiment as separate formulations, in any order. Agents capable of preventing and/or treating an infection with influenza virus and/or a condition resulting from such an infection that are in the experimental phase might also be used as other therapeutic and/or prophylactic agents useful in the present invention.

### Influenza B Vaccine

In various embodiments a vaccine is provided for preventing an influenza infection. Advantageously the vaccine can provide protection from Influenza B viruses. In various embodiments, the vaccine may comprise an Influenza B virus (IBV) neuraminidase epitope, such as, for example a polypeptide comprising the residues targeted by the antibodies or antigen-binding fragments described herein. For example, the epitope can comprise an amino acid sequence comprising at least about 70% sequence identity to SEQ ID NO: 149 and containing at least one of the residues selected from the group consisting of: R147, K435, R116, R292, R374, Y409, D149, E226 H134, R147, R116, and R374. In some cases, the epitope comprises at least about 70% identity to SEQ ID NO: 149 and comprises the following residues: R147, K435, R116, 8292, R374, Y409, D149, E226. In some cases, the epitope comprises at least about 70% identity to SEQ ID NO: 149 and comprises the following residues H134, R147, R116, and R374. In all of the epitopes described herein, residues are numbered according to the amino acid numbering of SEQ ID NO: 149.

In various embodiments, the vaccine further comprises an adjuvant to stimulate an immune response. Suitable adjuvants are known in the art and can include, for example, alum, aluminum hydroxide, monophosphoryl lipid A (MPL) or combinations thereof. Further, the vaccine may be prepared using suitable carriers and excipients according to pharmaceutical compositions described herein above.

In various embodiments, the vaccine can elicit an immunological response to prevent an influenza infection. The influenza infection may be caused by an influenza B virus. In various embodiments, the influenza B virus belongs to the B/Yamagata/16/88-like lineage or the B/Victoria/2/87-like lineage.

### Methods of Treating

In various embodiments, a method of preventing or treating influenza in a subject in need thereof is provided. The method can comprise administering any antibody or antigen-binding fragment (including any nucleic acid or expression vector that encodes the antibody or antigen-binding fragment), any vaccine, or any composition as described herein to the subject.

In various embodiments, the composition is administered parentally (e.g., systemically). In other embodiments, the composition is inhaled orally (e.g., intranasally). In both cases the composition is formulated (e.g., with carriers/excipients) according to its mode of administration as described above.

In various embodiments the composition is administered via intranasal, intramuscular, intravenous, and/or intradermal routes. In some embodiments, the composition is provided as an aerosol (e.g., for nasal administration).

Dosing regiments can be adjusted to provide the optimum desired response (e.g., a prophylactic or therapeutic response). Therefore, the dose used in the methods herein can vary depended on the intended use (e.g., for prophylactic vs. therapeutic use). Nevertheless, the compositions described herein may be administered at a dose of about 1 to about 100 mg/kg body weight, or from about 1 to about 70 mg/kg body weight. Furthermore, a single bolus may be administered, several divided doses may be administered over time, or the dose may be proportionally reduced or increased as indicated by the exigencies of the therapeutic of the therapeutic situation.

In various embodiments, the antibody or antigen-binding fragment is delivered using a gene therapy technique. Such techniques are well known in the art and generally comprise administering a viral vector comprising a nucleic acid that codes for a gene product of interest to a subject in need thereof. Therefore, in certain embodiments, the antibody or antigen-binding fragment described herein is delivered to a subject in need thereof by administering a viral vector or vectors (e.g., an adenovirus) containing one or more of the necessary nucleic acids (such as, for example, the nucleic acids provided herein) for expressing the antibody or antibody binding fragment in vivo. Similar delivery methods have successfully lead to the expression of protective antibodies in other disease contexts. For example, see Sofer-Podesta C. et al., “Adenovirus-mediated delivery of an Anti-V Antigen Monoclonal Antibody Protects Mice against a Lethal Yersinia pestis Challenge” Infection and Immunity March 2009, 77 (4) 1561-1568, the entire disclosure of which is incorporated herein by reference.

In various embodiments, the influenza to be treated is an influenza B virus. In some embodiments, the influenza B virus to be treated belongs to the B/Yamagata/16/88-like lineage or the B/Victoria/2/87-like lineage. For example, the influenza B virus can be a virus of the following strains: B/Phuket/3073/13(Y), B/Brisbane/60/08/(V), B/New York/PV0094/17(Y), B/New York/PB01181/18(V) or any other strain belonging to these lineages.

### Definitions

As used herein, the term “antigen-binding fragment” means any antigen-binding fragment of an antibody, including an intact antibody or antigen-binding fragment that has been modified, engineered or chemically conjugated. Examples of antibodies that have been modified or engineered are chimeric antibodies, humanized antibodies, and multispecific antibodies (e.g., bispecific antibodies. Antigen-binding fragments include, inter alia, Fab, F(ab′), F(ab′)2, Fv, dAb, Fd, complementarity determining region (CDR) fragments, single-chain antibodies (scFv), bivalent single-chain antibodies, single-chain phage antibodies, diabodies, triabodies, tetrabodies, (poly)peptides that contain at least a fragment of an immunoglobulin that is sufficient to confer specific antigen binding to the (poly)peptide, etc.). Regardless of structure, the antigen-binding fragment binds with the same antigen that is recognized by the intact immunoglobulin. An antigen-binding fragment can comprise a peptide or polypeptide comprising an amino acid sequence of at least 2, 5, 10, 15, 20, 25, 30, 35, 40, 50, 60, 70, 80, 90, 100, 125, 150, 175, 200, or 250 contiguous amino acid residues of the amino acid sequence of the binding molecule. The above fragments may be produced synthetically or by enzymatic or chemical cleavage of intact immunoglobulins or they may be genetically engineered by recombinant DNA techniques. The methods of production are well known in the art and are described, for example, in Antibodies: A Laboratory Manual, Edited by: E. Harlow and D, Lane (1988), Cold Spring Harbor Laboratory, Cold Spring Harbor, N.Y.

The term “complementarity determining regions” (CDR) as used herein means sequences within the variable regions of antibodies that usually contribute to a large extent to the antigen binding site which is complementary in shape and charge distribution to the epitope recognized on the antigen. The CDR regions can be specific for linear epitopes, discontinuous epitopes, or conformational epitopes of proteins or protein fragments, either as present on the protein in its native conformation or, in some cases, as present on the proteins as denatured, e.g., by solubilization in SDS. Epitopes may also consist of posttranslational modifications of proteins.

“Influenza A virus” as used herein refers to a type of influenza virus that can be further characterized into different “subtypes” that are characterized by various combinations of the hemagglutinin (H) and neuraminidase (N) viral surface proteins. There are 18 different hemagglutinin subtypes and 11 different neuraminidase subtypes (H1 through H18 and N1 through N11) of Influenza A viruses. Influenza A virus subtypes can be referred to by their H number, such as for example “influenza virus comprising HA of the H1 or H5 subtype”, or “H1 influenza virus” “H5 influenza virus”, or by reference to their N number, such as for example “influenza virus comprising NA of the N1 or N2 subtype”, or by a combination of a H number and an N number, such as for example “influenza virus subtype “H5N1 or H3N2”. The term influenza virus “subtype” specifically includes all individual influenza virus “strains” within each subtype, which usually result from mutations and show different pathogenic profiles. Such strains may also be referred to as various “isolates” of a viral subtype. Accordingly, as used herein, the terms “strains” and “isolates” may be used interchangeably. The current nomenclature for human influenza virus strains or isolates includes the geographical location of the first isolation, strain number and year of isolation, usually with the antigenic description of HA and NA given in brackets, e.g. A/Moscow/10/00 (H3N2). Non-human strains also include the host of origin in the nomenclature.

“Influenza B virus” as used herein, refers to a second category (type) of influenza virus. Unlike influenza A, influenza B viruses are not divided into subtypes but can be broken down into lineages and strains (e.g., B/Yamagata and B/Victoria). However, influenza B viruses do contain hemagglutinin and neuraminidase proteins which are classified herein as “Type B hemagglutinin” or “Influenza B virus hemagglutin” (IBV HA) and “Type B neuraminidase” or “Influenza B virus neuraminidase” (IBV NA), respectively.

The term “host”, as used herein, is intended to refer to an organism or a cell into which a vector such as a cloning vector or an expression vector has been introduced. The organism or cell can be prokaryotic or eukaryotic. Preferably, the hosts are isolated host cells, e.g. host cells in culture. The term “host cells” merely signifies that the cells are modified for the (over)-expression of the antibodies of the invention and include B-cells that originally express these antibodies and which cells have been modified to over-express the binding molecule by immortalization, amplification, enhancement of expression etc.

Amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

The term “operably linked” refers to two or more nucleic acid sequence elements that are usually physically linked and are in a functional relationship with each other. For instance, a promoter is operably linked to a coding sequence, if the promoter is able to initiate or regulate the transcription or expression of a coding sequence, in which case the coding sequence should be understood as being “under the control of” the promoter.

By “pharmaceutically acceptable excipient” is meant any inert substance that is combined with an active molecule such as a drug, agent, or antibody and that facilitate processing of the active compounds into preparations which can be used pharmaceutically. The “pharmaceutically acceptable excipient” is an excipient that is non-toxic to recipients at the used dosages and concentrations, and is compatible with other ingredients of the formulation comprising the drug, agent or binding molecule. Pharmaceutically acceptable excipients are widely applied and known in the art.

As used herein, the term “pharmaceutically acceptable carrier” means a non-toxic, inert solid, semi-solid or liquid filler, diluent, encapsulating material, or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil; and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring, and perfuming agents, preservatives and antioxidants can also be present in the composition, according to the judgment of the formulator based on the desired route of administration.

The term “specifically binding”, as used herein, in reference to the interaction of an antibody, and its binding partner, e.g. an antigen, means that the interaction is dependent upon the presence of a particular structure, e.g. an antigenic determinant or epitope, on the binding partner. In other words, the antibody preferentially binds or recognizes the binding partner even when the binding partner is present in a mixture of other molecules or organisms. The binding may be mediated by covalent or non-covalent interactions or a combination of both. In yet other words, the term “specifically binding” means immunospecifically binding to an antigenic determinant or epitope and not immunospecifically binding to other antigenic determinants or epitopes. An antibody that immunospecifically binds to an antigen may bind to other peptides or polypeptides with lower affinity as determined by, e.g., radioimmunoassays (RIA), enzyme-linked immunosorbent assays (ELISA), BIACORE, or other assays known in the art. Antibodies or fragments thereof that immunospecifically bind to an antigen may be cross-reactive with related antigens, carrying the same epitope. Preferably, antibodies or fragments thereof that immunospecifically bind to an antigen do not cross-react with other antigens.

The term “neutralizing” as used herein in relation to the antibodies of the invention refers to antibodies that inhibit an influenza virus from replication, in vitro and/or in vivo, regardless of the mechanism by which neutralization is achieved, or assay that is used to measure the neutralization activity.

The term “therapeutically effective amount” refers to an amount of the antibodies as defined herein that is effective for preventing, ameliorating and/or treating a condition resulting from infection with an influenza virus (e.g., influenza B). Amelioration as used herein may refer to the reduction of visible or perceptible disease symptoms, viremia, or any other measurable manifestation of influenza infection.

The term “treatment” refers to therapeutic treatment as well as prophylactic or preventative measures to cure or halt or at least retard disease progress. Those in need of treatment include those already inflicted with a condition resulting from infection with influenza virus as well as those in which infection with influenza virus is to be prevented. Subjects partially or totally recovered from infection with influenza virus might also be in need of treatment. Prevention encompasses inhibiting or reducing the spread of influenza virus or inhibiting or reducing the onset, development or progression of one or more of the symptoms associated with infection with influenza virus.

The term “vector” denotes a nucleic acid molecule into which a second nucleic acid molecule can be inserted for introduction into a host where it will be replicated, and in some cases expressed. In other words, a vector is capable of transporting a nucleic acid molecule to which it has been linked. Cloning as well as expression vectors are contemplated by the term “vector”, as used herein. Vectors include, but are not limited to, plasmids, cosmids, bacterial artificial chromosomes (BAC) and yeast artificial chromosomes (YAC) and vectors derived from bacteriophages or plant or animal (including human) viruses. Vectors comprise an origin of replication recognized by the proposed host and in case of expression vectors, promoter and other regulatory regions recognized by the host. A vector containing a second nucleic acid molecule is introduced into a cell by transformation, transfection, or by making use of viral entry mechanisms. Certain vectors are capable of autonomous replication in a host into which they are introduced (e.g., vectors having a bacterial origin of replication can replicate in bacteria). Other vectors can be integrated into the genome of a host upon introduction into the host, and thereby are replicated along with the host genome.

The term “structurally similar” as it relates to a polypeptide (e.g., an antibody or antigen-binding fragment thereof) refers to a polypeptide or protein that has one or more conservative substitutions and/or chemical modifications relative to the reference polypeptide but that retains the overall secondary, tertiary and/or quaternary structure of the reference polypeptide or protein. A polypeptide or protein “structurally similar” to another polypeptide or protein would be expected to have similar binding affinity to the reference protein's binding target.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

Having described the invention in detail, it will be apparent that modifications and variations are possible without departing from the scope of the invention defined in the appended claims.

### EXAMPLES

The following non-limiting examples are provided to further illustrate the present invention.

Materials and Methods

### Data and Code

The following materials and methods were used to perform the experiments described in the following Examples. The accession numbers for the mAbs generated in these experiments are GenBank: MN888992-MN889005, MT200637-MT200664. The accession numbers for the NA-1G05 structure are PDB: 6V4N and EMDB: EMD-21042. The accession numbers for the NA-2E01 structure are PDB: 6V40 and EMDB: EMD-21043. The data and code associated with these accession numbers are incorporated herein by reference.

Mice: Six- to eight-week-old female BALB/c mice were used for all animal experiments.

Patient: Human peripheral blood mononuclear cells (PBMCs) were obtained from a single subject enrolled into the Barnes Jewish Hospital Emergency Depaitment Influenza—EDFLU—a prospective observational cohort study in St. Louis, Mo. The patient was a 51 years old male recruited during the 2017-2018 influenza season and PBMCs were obtained on the 4th day of symptomatic illness following presentation for medical attention to the Barnes Jewish Hospital Emergency Department. The subject did not receive the 2017-2018 seasonal influenza virus vaccine but had received other seasonal influenza virus vaccines in previous influenza seasons. The subject was briefly admitted to the hospital and discharged 2½ days after admission without complications.

Cell Lines: Expi293F cells were grown in Expi293™ Expression Medium (#A1435102, Gibco). Madin Darby canine kidney (MDCK) cells were grown in Dulbecco's modified Eagle's medium (DMEM) supplemented with 5% FBS, penicillin (100 U/mL), and streptomycin (100 mg/mL). ADCC bioeffector FcgRIIIa cells (Promega) were thawed according to the manufacturer's protocol and used directly. Sf9 cells (#12659017, Gibco) were cultured in Sf-900 III SFM (#12658019, Gibco) supplemented with 0.5% penicillin-streptomycin (#15070063, GIBCO). High Five™ cells (#B85502, Gibco) were cultured in Express Five SFM (#10486025, Gibco) supplemented with 18 mM L-glutamine (#25030081, Gibco), 10 U/mL heparin (#H3149, Sigma-Aldrich), and 0.25% penicillin-streptomycin. Insect cells were maintained in an incubator at 28° C.

PBMC isolation: Blood was collected in ethylenediaminetetraacetic acid (EDTA)-anticoagulated sample tubes using standard phlebotomy techniques. PBMCs were prepared within 8 hours of collection by layering blood over Ficoll and centrifuging at 400 g for 30 minutes. The PBMC layer at the Ficoll interface was collected, washed with 1× phosphate-buffered saline (PBS) and resuspended in Roswell Park Memorial Institute (RPMI)-1640 media. Cell counts were obtained, and cells were cryogenically preserved in RPMI-1640 media supplemented with 10% dimethyl sulfoxide (DMSO) and 40% fetal bovine serum (FBS).

ELISpot: Direct ex vivo enzyme linked immunospot (ELISpot) was used to enumerate the number of IgG-secreting, recombinant HA-specific plasmablasts present in the PBMC sample. Basically, dilutions of washed PBMCs incubated in RPMI-1640 media [supplemented with 10% FBS, penicillin (100 U/mL) and streptomycin (100 μg/mL)] were incubated over 96-well ELISpot plates for 18 hours. After washing the plates with PBS supplemented with 0.05 Tween, secreted antibodies were detected with anti-human IgG-biotin (Jackson ImmunoResearch) and avidin-D-horseradish peroxidase (HRP) (Vector Laboratories) and developed with 3-amino-9-ethylcarbazole (AEC) substrate (Sigma) before analysis on an ELISpot counter (Cellular Technologies Ltd.).

Cell sorting: Staining for sorting was performed using cryo-preserved PBMCs resuspended in PBS supplemented with 2% FBS and 1 mM EDTA. Cells were stained for 30 minutes at 4° C. with CD71-FITC (clone CY1G4), CD19-PE (clone HIB19), CD38-BV605 (clone HIT2), CD20-APC-Fire750 (clone 2H7) and Zombie Aqua; all from Biolegend. Cells were then washed twice and single antibody secreting cells (ASCs) (live singlet CD19+CD38+CD71+) were sorted using a MoFlo (Beckman-Coulter) into 96-well plates containing 10 μL 10 mM Tris supplemented with 1 U/μL RNase inhibitor (Promega) and immediately frozen on dry ice.

Monoclonal antibody generation: Antibodies were cloned as previously described (Wrammert, et al. 2011, JEM 208 (1): 181). In brief, V_{H}, V_{λ}, and V_{κ} genes were amplified by reverse transcription polymerase chain reaction (RT-PCR) and nested PCR reactions from single-sorted ASCs using cocktails of primers specific for IgG, Igλ, and Igκ using primer sets detailed in (Smith et al. 2009, Nat. Protoc. 4:372-384) and then sequenced. To generate recombinant antibodies, PCR was performed with variable and junction gene primers containing short extensions to match the cloning site of the antibody expression vectors (overlap extension PCR) and the amplified fragments were cloned by Gibson assembly, as previously described (Ho et al. 2016, J. Imm Meth. 438:67-70). Heavy and light chain plasmids were co-transfected into Expi293F cells (Gibco) for expression and antibody was purified with protein A agarose (Invitrogen).

Cells, viruses and recombinant proteins: Expi293F cells were grown in Expi293 Expression medium (Gibco). Madin Darby canine kidney (MDCK) cells were grown in Dulbecco's modified Eagle's medium (DMEM) with 5% FBS, penicillin (100 U/mL) and streptomycin (100 μg/mL). ADCC bioeffector FcγRIIIa cells (Promega) were thawed according to the manufacturer's protocol and used directly. Influenza viruses were grown in 8- to 10-day old embryonated chicken eggs for 3 days at 37° C. (influenza A viruses) or 33° C. (influenza B viruses). Recombinant NA and HA proteins were expressed in the baculovirus expression system as previously described (Margine et al., 2013).

Enzyme-linked immunosorbent assay (ELISA): Ninety-six-well microtiter plates (Thermo Fisher Scientific) were coated with 100 mL inactivated virus diluted 1:100 in PBS or recombinant NA or HA proteins at a concentration of 1 mg/mL in PBS at 4° C. overnight. Wells were blocked with 280 mL PBS supplemented with 0.05% Tween-20 and 10% FBS, and plates were incubated for 1.5 h at room temperature (RT). The blocking solution was removed, and 1:30 and 1:90 dilutions of mAb transfection culture supernatant or 3-fold serial dilutions of purified mAbs were added. After incubating at RT for 1 h, the plates were washed 3 times with T-PBS. HRP-conjugated anti-human IgG (Jackson ImmunoResearch) was diluted 1:2500 in blocking solution and added to all wells (100 mL/well). The plates were incubated at RT for 1 h, and washed 3 times with T-PBS and 3 times with PBS. Then, 100 mL substrate solution [phosphate-citrate buffer with 0.1% H₂O₂ and 0.4 mg/mL o-phenylenediamine dihydrochloride (OPD, Sigma)] was added to all wells and incubated for 5 min. The reaction was stopped with 1 M hydrochloric acid (HCl) (100 mL/well). The plates were read at a wavelength of 490 nm with a microtiter plate reader (Bio-Tek). The data were analyzed using Microsoft Excel and GraphPad Prism 7.

Passive transfer experiments in mice: All animal experiments were conducted in accordance with institutional guidelines. Mouse passive transfer experiments were performed as previously described (Stadlbauer et al., 2018). In the prophylactic settings, 6- to 8-week-old female BALB/c mice were given 100 μL of each BNA-mAb individually at a concentration of 5 mg/kg or 1 mg/kg intraperitoneally (n=5 mice/mAb). Negative control mice received an irrelevant human IgG control mAb at the same dose. The mice were challenged intranasally with 5×LD₅₀ challenge virus 2 hours after the mAb transfer, while being deeply anesthetized with a ketamine/xylazine mixture. Survival and weight loss were monitored daily for 14 days, and animals that lost 25% or more of their initial body weight were euthanized. In the therapeutic setting, mice were infected with 5×LD₅₀ of B/New York/PV00094/17 and given a 5 mg/kg dose of mAb 72 hours post infection (n=5 mice/mAb). Survival and weight loss were monitored daily for 14 days, and animals that lost 25% or more of their initial body weight were euthanized. For determining the virus lung titers, mice received a 5 mg/kg dose of mAb. After two hours, mice were infected with 0.1×LD₅₀ of B/New York/PV00094/17. Lungs were harvested at day 3 (n=3 mice/mAb) and day 6 (n=3 mice/mAb) post infection. Lung virus titers were assessed by standard plaque assay (McMahon et al., 2019) and analyzed in Microsoft Excel and GraphPad Prism 7.

Enzyme-linked lectin assay: Ninety-six-well flat bottom microtiter plates (Thermo Fisher Scientific) were coated with 100 mL/well fetuin (Sigma) at a concentration of 25 mg/mL in 1×3 coating buffer (KPL coating solution, SeraCare) at 4° C. overnight. The next day, plates were washed 3 times with T-PBS. In a separate 96-well plate, mAbs were 2-fold serially diluted in sample diluent [PBS with 1% bovine serum albumin (BSA, Sigma) and 0.5% Tween-20 (Sigma)] starting at 30 mg/mL. Fifty microliters of each mAb dilution were transferred to the fetuin-coated plate in duplicate wells. Next, 50 mL of virus at a predetermined 90% maximal effective concentration (EC₉₀) were added to the plates and incubated at 37° C. for 18 h. The following day, the plates were washed 6 times, and 100 ml/well peanut agglutinin (PNA)-HRP (Sigma) was added at a concentration of 1 mg/mL in PBS with 1% BSA. After a 2-h incubation at RT, the plates were developed with 100 mL SigmaFast OPD. The reaction was stopped after 10 min by adding 50 mL 3M HCl (Thermo Fisher Scientific), and the plates were read at a wavelength of 490 nm with a microtiter plate reader (Bio-Tek). The data were analyzed using Microsoft Excel and GraphPad Prism 7, and the 50% inhibition concentration (IC₅₀) was defined as the concentration of mAb at which 50% of the NA activity was inhibited compared to the negative control (virus with no mAb).

Neuraminidase inhibition by NA-Star assay: The NA-Star® Influenza Neuraminidase Inhibitor Resistance Detection Kit (Applied Biosystems) was used to quantify the inhibition of NA activity (cleavage of a small chemiluminescent substrate) in the presence of NA-mAbs. The experiments were preformed according to the manufacturer's protocol. In short, mAbs were diluted in NA-Star Assay Buffer to a concentration of 100 μg/mL and subsequently serially diluted 1:3. Twenty-five microliters from each dilution were transferred to a white, flat bottom 96-well cell culture plate and mixed with 25 μL/well of B/Phuket/3073/2013 virus at a predetermined 2×EC₅₀ for 20 minutes at 37° C. NA-Star Substrate (10 μL/well) was added after the incubation and the plates were incubated at RT for 30 minutes. NA-Star accelerator solution (60 μL/well) was added to the plates immediately before the readout. The chemiluminescent signal was detected by a microtiter plate reader (Bio-Tek) and analyzed using Microsoft Excel and GraphPad Prism 7.

Plaque reduction: NA plaque reduction assays were performed as described previously (Wohlbold et al., 2017). In short, mAbs were 5-fold serially diluted in 1× Minimum Essential Medium (MEM) starting at 100 μg/ml and incubated with 50 μl of B/Phuket/3073 or B/Brisbane/60/08 virus at 2000 plaque forming units (PFU) per mL for 1 hour at RT on a shaker. The virus and mAb mixtures were plagued on a monolayer of MDCK cells in a 12-well plate and incubated at 33° C. for 3 days. After the incubation, cells were fixed with 3.7% formaldehyde for 1 hour at 4° C. and blocked with 3% milk in PBS for 1 hour at RT. The cells were then incubated with anti-IBV guinea pig sera diluted to 1:500 in PBS with 1% milk for 1 hour at RT. The plates were washed with PBS and incubated with secondary donkey anti-guinea pig IgG antibody conjugated to HRP (Millipore) for 1 hour at RT. The plates were washed, and plaques were visualized by staining with KPL True-Blue peroxidase (Sera Care). The plaques were counted at each dilution and compared to a no-antibody control. The data were analyzed in Microsoft Excel and GraphPad Prism 7.

ADCC reporter assay (Promega): A commercial ADCC reporter assay kit (Promega) was used to assess the ability of the mAbs to activate ADCC pathways. Briefly, 100 pt/well MDCK cells (2×105 cells/mL) in RPMI 1640 media were seeded into white, flat bottom, 96-well cell culture plates (Corning) and incubated overnight at 37° C. The next day, cells were infected with B/Phuket/3073/13 or B/Brisbane/60/08 virus at a multiplicity of infection (MOI) of 3 and incubated at 33° C. After 16 hours, the media was replaced with human ADCC bioeffector FcγRIIIa cells (Promega) and 3-fold serial dilutions of mAbs in assay buffer (starting at 30 μg/mL). After a 6-hour incubation at 37° C., Bio-Glo™ luciferase (Promega) was added to each well and incubated for 10 minutes in the dark at RT. The luminescence was measured by a microtiter plate reader (Bio-Tek), and the data were analyzed using Microsoft Excel and GraphPad Prism 7.

Competition ELISA: Ninety-six-well flat bottom microtiter plates (Thermo Fisher) were coated with 50 μL of 5 μg/mL purified B/New York/PV00094/17 (Y) virus diluted in coating solution (KLP) and incubated at 4° C. overnight. On the following day, the plates were washed three times with T-PBS and incubated for 1 hour at RT with 200 μL/well blocking solution (PBS-T with 3% goat serum (Life Technologies, Inc.) and 0.5% milk powder). Next, the blocking solution was discarded and unbiotinylated competing mAbs (100 μL/well) were added to the plates at a concentration of 20 μg/mL in blocking solution. Blocking solution with no mAbs was used as a negative control. The plates were incubated for 2 hours at RT and subsequently washed 3 times with T-PBS. A second set of the mAbs (target mAbs) was labeled with biotin using the EZ-Link NHS-PEG4-Biotin kit (Thermo Fisher Scientific) according to the manufacturer's instructions. The biotinylated target mAbs were serially diluted 1:3 starting at 30 μg/mL in blocking solution and transferred to the 96-well plate with the competing mAbs (100 pt/well). The plates were incubated for 2 hours at RT and washed 3 times with T-PBS. The plates were subsequently incubated for 1 hour at RT with 50 μL/well streptavidin conjugated to HRP (Thermo Fisher Scientific) diluted 1:3000 in blocking solution. After the incubation, the plates were washed 4 times with T-PBS and developed with 100 μL SigmaFast OPD. The reaction was stopped after 10 minutes by adding 50 μL 3M HCl (Thermo Fisher) and the plates were read at a wavelength of 490 nm with a microtiter plate reader (Bio-Tek). The data were analyzed using Microsoft Excel and GraphPad Prism 7. The level of binding was measured as area under the curve. The percent competition for each mAb was calculated as the reduction in binding relative to the level of inhibition of any particular mAb against itself.

Cloning, expression and purification of 1G05, 1D05 and 2E01 Fabs: The VDJ region of the antibody sequences were subcloned with AgeI and SalI restriction endonucleases from pAbVec6W-hIgG1 to a modified pAbVec6W vector for Fab expression in which the encoded C-terminus of the hIgG1 constant region was replaced with a thrombin cleavage site and 6×HIS tag. After 6 days of transfection, the cell culture supernatant was harvested and dialyzed against buffer 20 mM Tris-Cl, 150 mM NaCl, pH 8.0. Fabs were captured by passaging over Ni2+ affinity resin and eluted in 500 mM imidazole. The elute was then sized with HiLoad 16/600 Superdex 200 column (GE healthcare) in 20 mM Hepes, 150 mM NaCl, pH 7.4 with Fab fractions pooled and concentrated.

Cloning, expression and purification of B/Phuket/3073/2013 NA for structural studies: The ectodomain of NA from B/Phuket/3073/2013 (EPI529344) was expressed using the flashBAC baculovirus expression system (Minis) according to the manufacture's protocol. Briefly, NA ectodomain residues W80-L466 were fused with an N-terminal gp67 signal peptide, a His-tag, and the human vasodilator-stimulated phosphoprotein tetramerization domain with a thrombin cleavage site (Xu et al., 2008). This construct was cloned into a modified pOET1 transfer vector containing green fluorescent protein as an indicator. The construct was co-transfected with flashBAC DNA into sf9 insect cells to generate the corresponding baculovirus. Suspension cultured Hi5 cells were infected at a density of 1.5×106 cells/ml with P2 virus at MOI of 1-5. The cell culture supernatant was harvested 72-hr post-infection and secreted NA protein was further purified by Ni²⁺ affinity chromatography and size exclusion chromatography.

Binding Affinity Measurement with Bio-Layer Interferometry: The binding affinity of NA with 1G05 and 2E01 Fabs was measured by BLI with Octet-Red96 instrument (ForteBio) as described previously (Ellebedy et al., 2020). The NA tetramer was randomly biotinylated (EZ-Link-NHS-PEG4-Biotin, Thermo Fisher), and excess biotin was removed by a desalting column (0.5 mL Zeba Spin 7K MWCO, Thermo Fisher). Briefly, for BLI monitoring, the biotinylated NA protein were loaded onto streptavidin biosensors (ForteBio), at 5 mg/mL for 2 min in HBS-EP buffer (10 mM HEPES pH 7.4, 150 mM NaCl, 3 mM EDTA, and 0.005% P20 surfactant) with 1% BSA. Five 3-fold serial dilutions of Fab samples were used per kinetics assay. The real-time data of BLI were recorded at 25° C. and processed using Biaevaluation 3.1 (GE Healthcare). The 1:1 binding model was employed for the association and dissociation rate constants analyses and steady-state equilibrium concentration curves fitting. The t_{1/2} value of each Fab was then calculated using the formula t_{1/2}=ln 2/K_{d}, where K_{d} represents the dissociation rate constant.

Cryo-EM data acquisition and image processing: Purified NA at 1 mg/ml was incubated with Fabs at 2 mg/ml at a molar ratio of 1:1 in buffer 25 mM Hepes pH 7.4, 150 mM. For both samples, a 3 μl volume of the mixture was then applied to glow-discharged holey carbon-coated grids (Quantifoil) and flash frozen in liquid ethane using a FEI Vitrobot (Thermo Fisher). The grids were imaged on a Titan Krios (Thermo Fisher) microscope operating at 300 keV using Gatan K2 Summit detector with a total electron dose of 66 e-Å-2. Images were collected with 200 ms frame time over 40 frames in counting mode with a calibrated pixel size of 1.1 Å and a slit width of 20 eV at 105,000× magnification. Cryo-EM data processing was mainly carried out using RELION 3.0-beta-2 (Zivanov et al., 2018). In detail, frame alignment was carried out using MotionCor2 (Zheng et al., 2017) with dose weighting and contrast transfer function (CTF) estimation performed using Gctf (Zhang, 2016). For dataset NA-1G05, a total of 385,417 particles were extracted from 1653 micrographs using crYOLO (Wagner et al., 2019). Reference-free two-dimensional (2D) classification was used to select 13 classes containing 240,330 good particles. 3D classification with good 2D-classes was then carried out using a H11N9 NA-single chain antibody complex structure (PDB code 1A14) low pass filtered to 60 Å as a reference. The best classes containing 159,589 particles were then used in 3D consensus refinement, followed by CTF parameter refinement, Bayesian polishing and further 3D consensus refinement. A final “gold standard” refinement produced the final map with a resolution of 2.5 Å after PostProcess masking (B factor sharpening) (Chen et al., 2013). Local resolution estimates were calculated with ResMap (Kucukelbir et al., 2014). The data processing of dataset NA-2E01 was similar to that of NA-1G05. Briefly, a total of 459,004 particles were extracted from 1660 micrographs using crYOLO. After 2D-classification, 37 classes of 301,384 particles were selected for 3D-classification. Then the two best classes containing 150,730 particles were used for further refinement, leading to a final map with a resolution of 2.8 Å after PostProcess masking.

Atomic model building, refinement and analysis: For NA-1G05, crystal structure B/Beijing/1/1987 NA (PDB code: 1NSC) and a Fab that binds to HA (PDB code: 6BTJ) were fitted into the Cryo-EM map using UCSF Chimera (Pettersen et al., 2004), followed by rigid-body refinement using Phenix (Adams et al., 2010). Model building was carried out manually in Coot (Emsley and Cowtan, 2004). The NA-1G05 model was refined to a final resolution of 2.5 Å using real space refinement in PHENIX and was assessed using MolProbity (Chen et al., 2010). The final model has a real space correlation coefficient of 0.83 and contains residues W80-L466 for NA and 182 solvent molecules. For NA-2E01, the model of NA-1G05 was fit into the Cryo-EM map, and model building and refinement were performed using Coot and Phenix. The final model of NA-2E01 has a real space correlation coefficient of 0.83 and four regions from NA were unable to be built, residues E105-S110, G140-Y143, G433-T436 and L455-L466, due to high flexibility. Structural figures were prepared using UCSF Chimera, UCSF ChimeraX (Goddard et al., 2018) and PyMOL (Schrödinger).

BNA-mAb Sequences: Sequences were obtained from PCR reaction products and annotated using the IMGT/V-QUEST database tool (www.imgt.org/IMGT_vquest/input) (Brochet et al., 2008; Giudicelli et al., 2011).

NA Sequences: The NA sequences for generating the phylogenetic tree were downloaded from the Global Initiative on Sharing Avian Influenza Data (www.gisaid.org). The amino acid sequences were aligned in Clustal Omega (Sievers and Higgins, 2014), and the phylogenetic tree was generated using MEGA 6.06 (Tamura et al., 2013).

### Example 1: Isolation of Broadly Cross-Reactive Anti-NA Monoclonal Antibodies

Peripheral blood samples were obtained from a hospitalized patient with confirmed IBV infection on day 4 after onset of symptomatic illness during the 2017-18 influenza season.

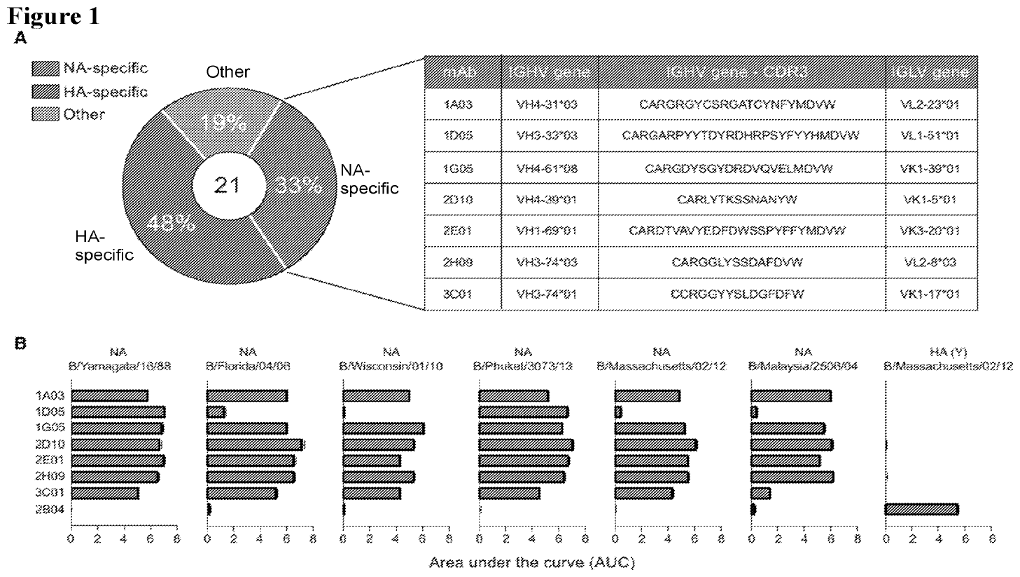

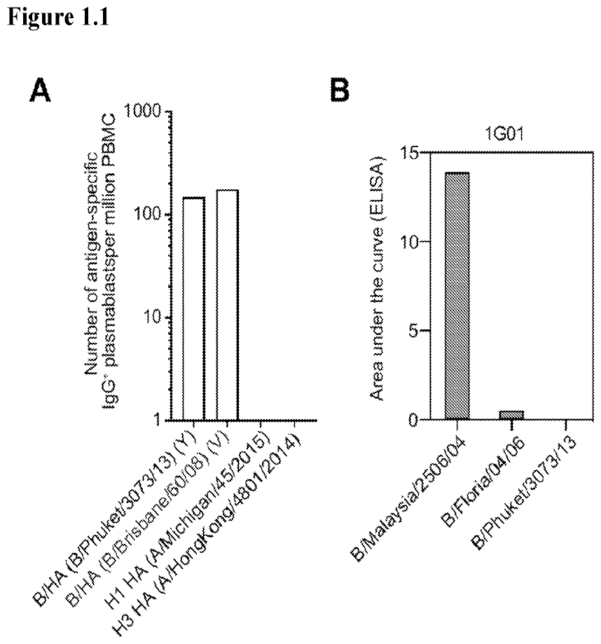

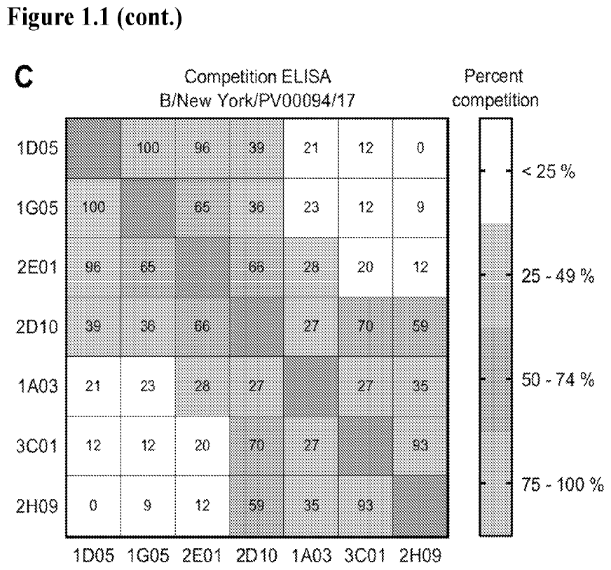

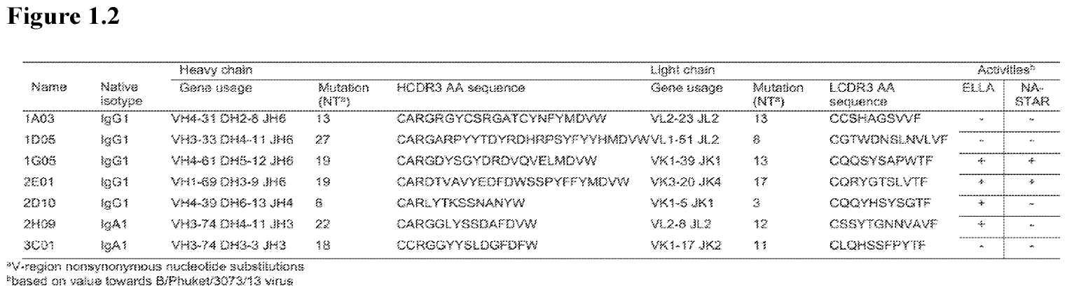

IBV infection was consistent with an HA-specific plasmablast response that was exclusively directed against IBV HAs rather than HAs derived from IAV H1N1 or H3N2 influenza virus strains as measured by enzyme-linked immunospot (ELISpot) assay (FIG. 1.1, A). Plasmablasts (defined as CD19⁺ IgD⁻ CD38⁺ CD20⁻ CD71^{hi}) were single-cell sorted, and the corresponding mAbs were expressed (Ellebedy et al., 2016; Wrammert et al., 2011). A total of 21 recombinant clonally distinct mAbs specific against IBV were generated (FIG. 1, A). Further screening revealed that ten of these mAbs recognized recombinant IBV HA, seven were IBV NA-specific, and the remaining four were directed against IBV NP and M1 proteins (FIG. 1, A). The seven anti-NA mAbs were derived from distinct B cell clones (Table 9, FIG. 1.2). Further evaluation of the anti-NA-mAbs revealed broad cross-reactivity to recombinant NA proteins from both the B/Yamagata/16/88-like and the B/Victoria/2/87-like lineages (FIG. 1, B; Table 10). By contrast, the broadly neutralizing anti-IAV NA-mAb 1G01 (Stadlbauer et al., 2019) displayed binding to a very limited number of BNA, such as NA from B/Malaysia/2506/04 (FIG. 1.1, B). Competition ELISA among the BNA-mAbs indicated that 1G05, 1D05, and 2E01 recognized potentially overlapping epitopes within the B/New York/PV00094/17 NA (FIG. 1.1, C). Similarly, mAbs 3C01 and 2H09 strongly inhibited each other's binding (>90%), indicating a potentially common epitope. The mAbs 3C01 and 2H09 share the same heavy chain variable gene (VH3-74), but not the light chain variable gene (FIG. 1, A; Table 9; FIG. 1.2). These data indicated that IBV infection elicited a robust and cross-reactive plasmablast response to NA.

TABLE 9
Immunoglobulin gene usage of the BNA-mAbs

| | | | D- | CDR- | | SEQ |
|---|---|---|---|---|---|---|
| | V- | J- | gene | lengths | | ID |
| Name | gene | gene | | (1.2.3) | AA junction | NO: |
| 1A03- | IGHV4- | IGHJ6* | IGHD2- | 10.7.20 | CARGRGYCSRGA | 123 |
| HC | 31*03 | 03 | 8*01 | | TCYNFYMDVW | |
| 1A03- | IGKV3- | IGKJ3* | | 6.3.8 | CCSHAGSVVF | 130 |
| LC | 11*01 | 01 | | | | |
| ID05- | IGHV3- | IGHJ6* | IGHD4- | 8.8.25 | CARGARPYYTDYRD | 125 |
| HC | 33*03 | 03 | 11*01 | | HRPSYFYYHMDVW | |
| 1D05- | IGLV1- | IGLJ2* | | 8.3.11 | CGTWDNSLNVLVF | 132 |
| LC | 51*02 | 01 | | | | |
| 1G05- | IGHV4- | IGHJ6* | IGHD5- | 8.7.19 | CARGDYSGYDRDV | 121 |
| HC | 61*08 | 03 | 12*01 | | QVELMDVW | |
| 1G05- | IGKV1- | IGKJl* | | 6.3.9 | CQQSYSAPWTF | 128 |
| LC | 39*01 | 01 | | | | |
| 2D10- | IGHV1- | IGHJ6* | IGHD3- | 8.8.23 | CARDTVAVYEDFD | 122 |
| HC | 69*01 | 03 | 9*01 | | WSSPYFFYMDVW | |
| 2D10- | IGKV3- | IGKJ4* | | 7.3.9 | CQRYGTSLVTF | 129 |
| LC | 20*01 | 01 | | | | |
| 2E01- | IGHV4- | IGHJ4* | IGHD6- | 10.7.12 | CARLYTKSS | 124 |
| HC | 39*01 | 02 | 13*01 | | NANYW | |
| 2E01- | IGKV1- | IGKJl* | | 6.3.9 | CQQYHSYSGTF | 131 |
| LC | 5*01 | 01 | | | | |
| 2H09- | IGHV3- | IGHJ3* | IGHD4- | 8.8.13 | CARGGLYSSDAFD | 126 |
| HC | 74*03 | 01 | 11*01 | | VW | |
| 2H09- | IGLV2- | IGLJ2* | | 9.3.10 | CSSYTGNNVAVF | 133 |
| LC | 8*03 | 01 | | | | |
| 3C01- | IGHV3- | IGHJ3* | IGHD3- | 8.8.13 | CCRGGYYSLDGFD | 127 |
| HC | 74*01 | 01 | 3*01 | | FW | |
| 3C01- | IGKV1- | IGKJ2* | | 6.3.9 | CLQHSSFPYTF | 134 |
| LC | 17*01 | 01 | | | | |

TABLE 10
Information on the influenza virus strains
and recombinant proteins used in the study.

| Virus strain names | Subtype |
|---|---|
| B/Lee/1940 | B |
| B/Yamagata/16/88 | B (Y) |
| B/Florida/4/06 | B (Y) |
| B/Wisconsin/01/10 | B (Y) |
| B/Massachusets/02/12 | B (Y) |
| B/Phuket/3073/13 | B (Y) |
| B/New York/PV00094/17 | B (Y) |
| B/Victoria/02/1987 | B (V) |
| B/Malaysia/2506/04 | B (V) |
| B/Brisbane/60/08 | B (V) |
| B/New York/PV00081/18 | B (V) |
| NA (B/Yamagata/16/88) | B (Y) |
| NA (B/Florida/4/06) | B (Y) |
| NA (B/Wisconsin/01/10) | B (Y) |
| NA (B/Massachusets/02/12) | B (Y) |
| NA (B/Phuket/3073/13) | B (Y) |
| NA (B/Malaysia/2506/04) | B (V) |
| HA (B/Massachusets/02/12) | B (Y) |
| HA (B/Phuket/3073/13) | B (Y) |
| HA (B/Brisbane/60/08) | B (V) |
| HA (A/Michigan/45/15) | H1N1 |
| HA (A/HongKong/4801/14) | H3N2 |
| NA (B/Colorado/06/17) | B (V) |
| NP (B/Brisbane/60/08) | B (V) |
| M1 (B/Brisbane/60/08) | B (V) |

### Example 2: Anti-BNA mAbs Exhibit Broad Enzyme Inhibition and IBV Neutralization In Vitro

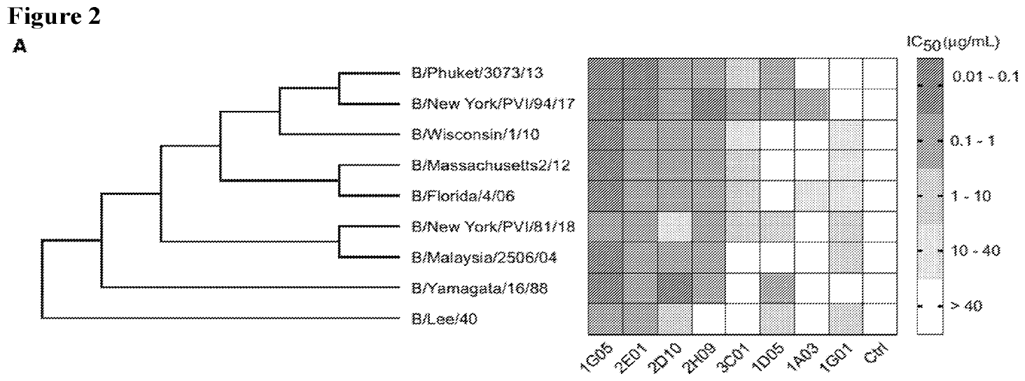

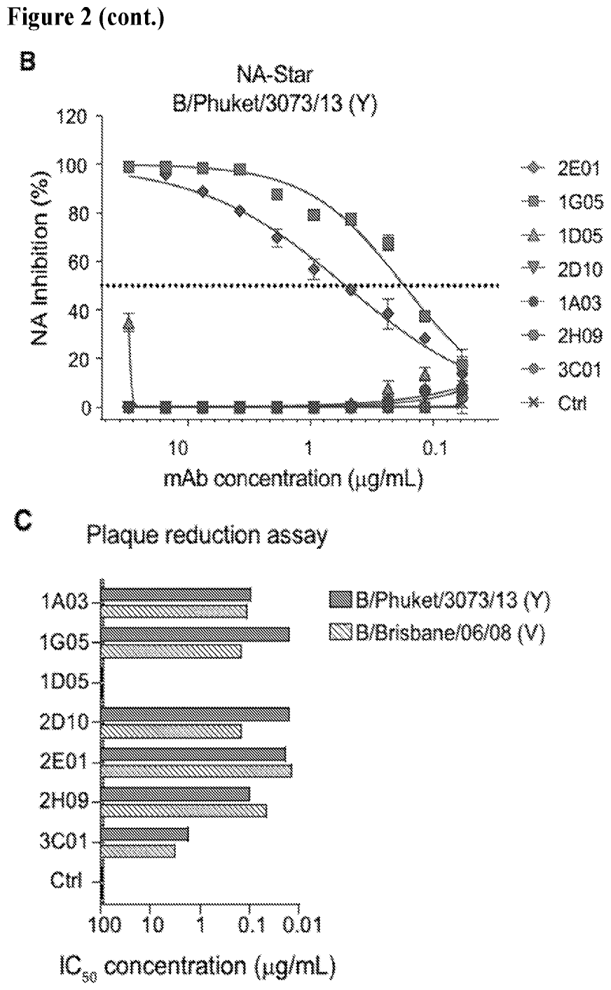

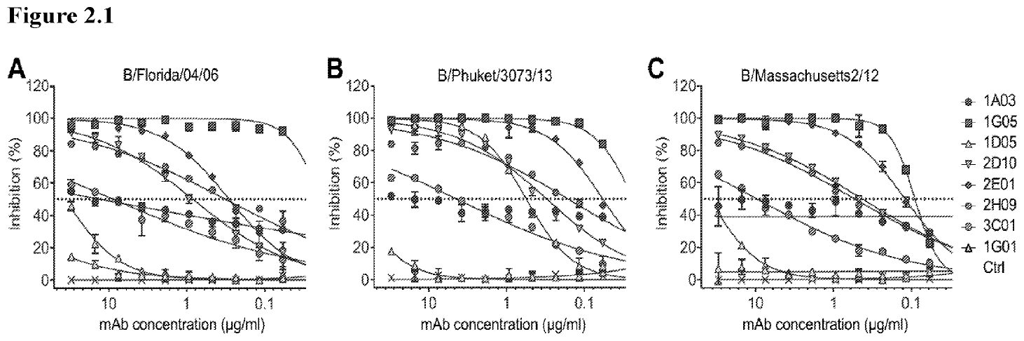

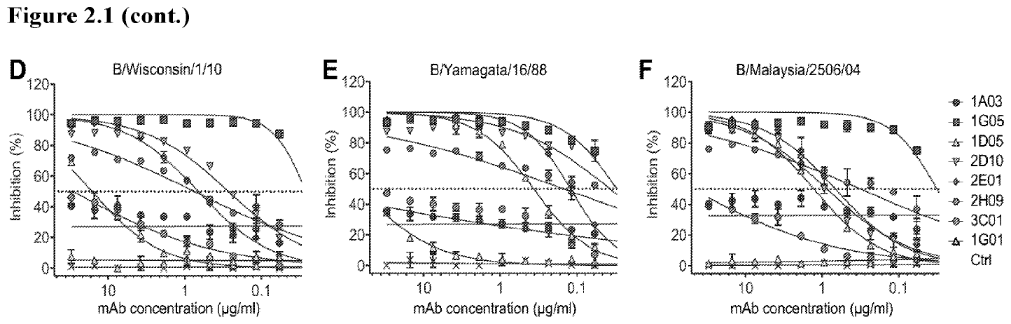

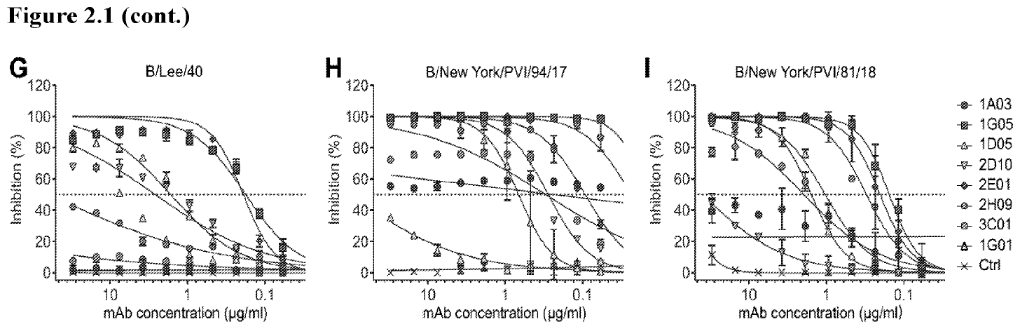

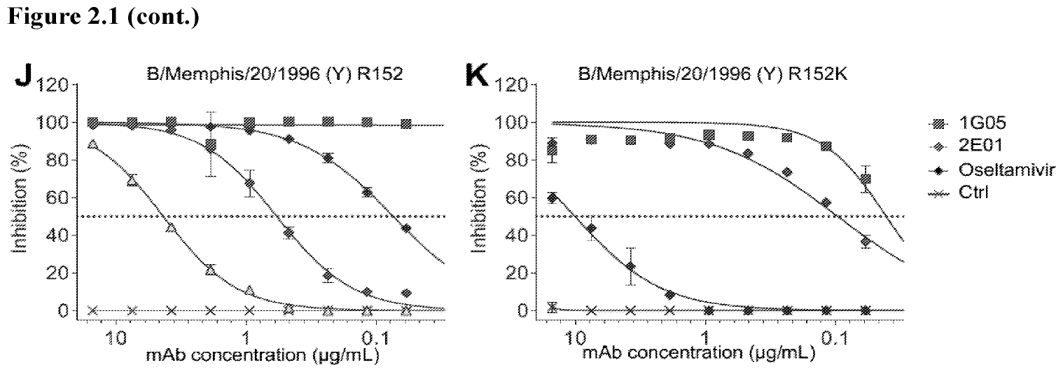

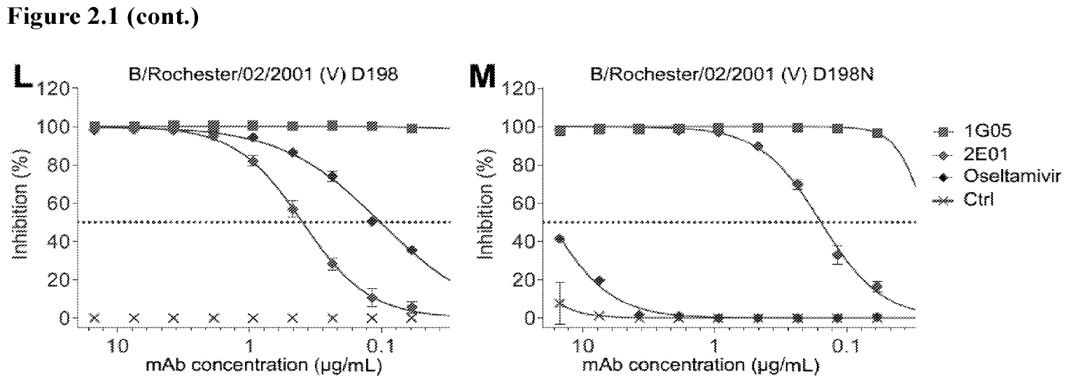

The BNA-mAbs were further characterized in an enzyme-linked lectin assay (ELLA) to better assess their potential to inhibit the enzymatic activity of NA (FIG. 2, A; FIGS. 2.1, A-I). All mAbs showed some NA inhibition (NI) activity, and 1G05 and 2E01 demonstrated remarkable NI activities against viruses belonging to the B/Yamagata/16/88-like and B/Victoria/2/87-like lineages and the ancestral B/Lee/1940 strain, which cumulatively span more than 70 years of antigenic drift (FIG. 2, A). We further examined the NI capacity of 1G05 and 2E01 against two zanamivir- and oseltamivir-resistant IBV strains, B/Memphis/20/1996 (Y) R152K (Gubareva et al., 1998) and B/Rochester/02/2001 (V) D198N (Ison et al., 2006) and their wild-type counterparts, using oseltamivir as a control. The NI activity of oseltamivir was severely diminished against the resistant mutants, whereas the NI activities of both 1G05 and 2E01 were minimally impacted (FIG. 2.1, J-M). NA enzymatic activity can be inhibited by mAbs binding directly or proximal to the enzymatic active site through steric hindrance. ELLA uses a large substrate (fetuin), which can be blocked by steric hindrance. The NA-Star assay uses a smaller substrate, and enzymatic activity is inhibited only by mAbs that bind directly to the enzymatic active site (Chen et al., 2018; Wohlbold et al., 2017). Only BNA-mAbs 1G05 and 2E01 exhibited NI activity in the NA-Star assay (FIG. 2, B).

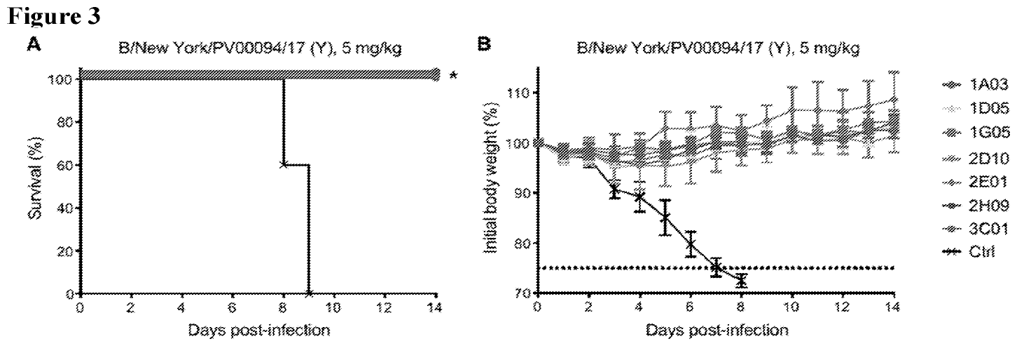

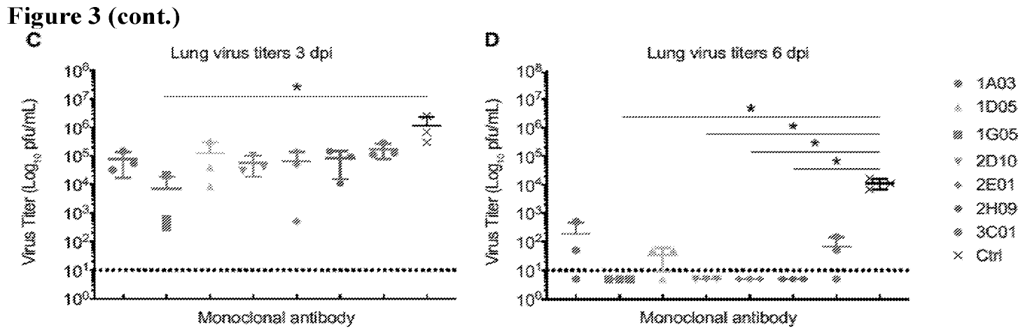

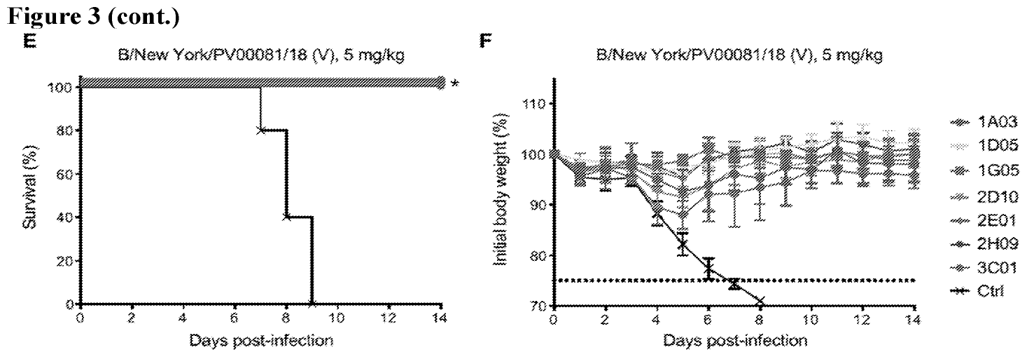

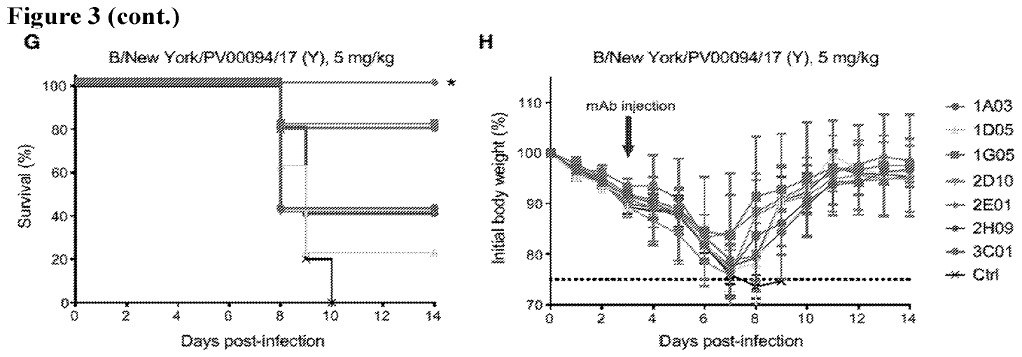

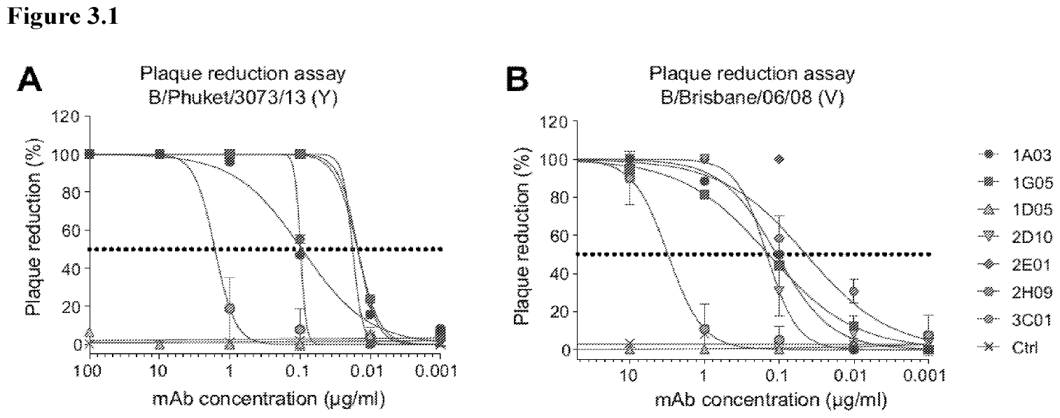

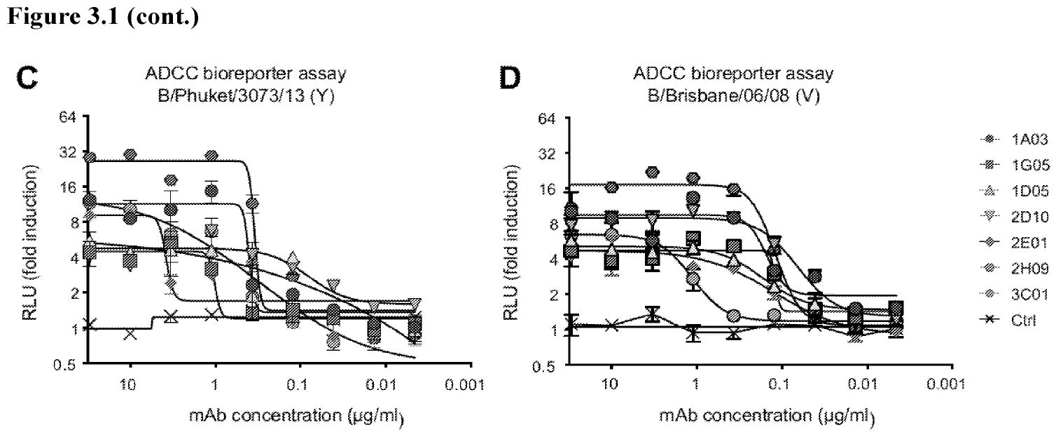

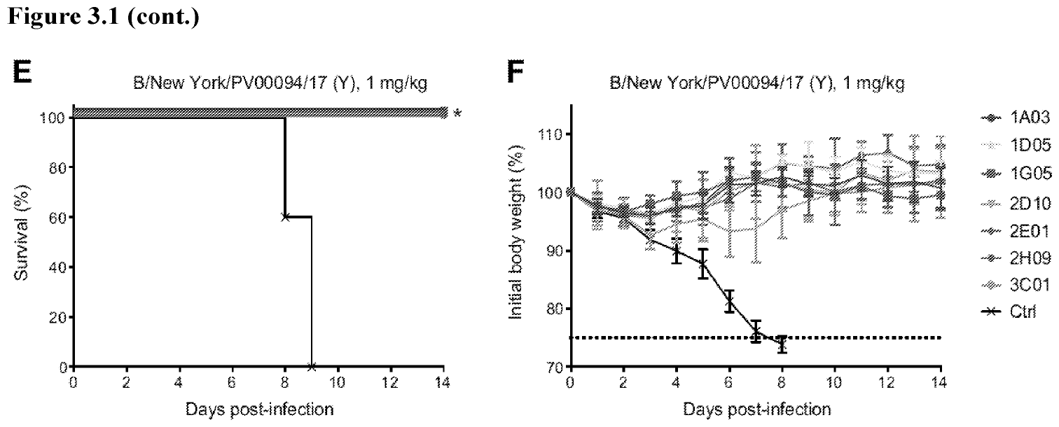

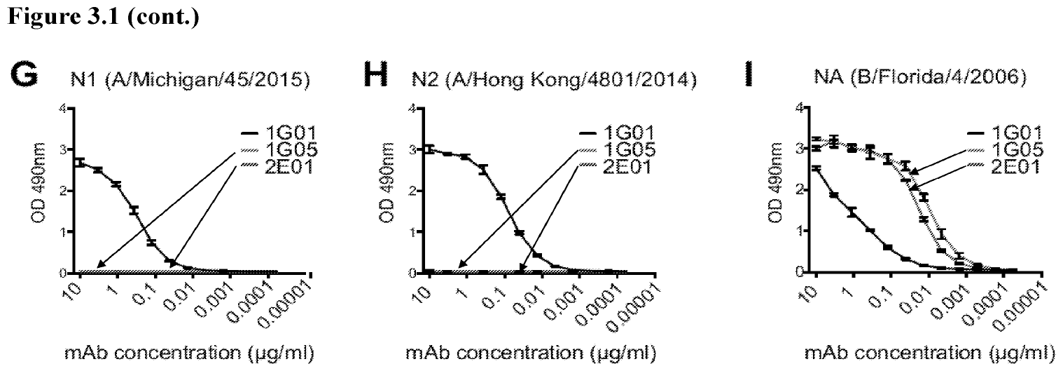

We tested whether the BNA-mAbs inhibited virus replication in vitro using a plaque reduction neutralization assay (PRNA). All BNA-mAbs except for 1D05 exhibited, to varying degrees, neutralizing activity against B/Phuket/3073/13 (Y) and B/Brisbane/60/08 (V) viruses (FIG. 2, C; FIG. 3.1, A; and FIGS. 3.1, B), and 1G05 and 2E01 were the most potent. Anti-influenza virus antibodies can mediate protection through Fc-receptor-mediated effector functions [e.g., antibody-dependent cellular cytotoxicity (ADCC)] (DiLillo et al., 2014; Wohlbold et al., 2017). All BNA-mAbs displayed activity in an ADCC reporter assay against B/Phuket/3073/13 (Y) and B/Brisbane/60/08 (V) viruses (FIG. 3.1, C and D). These combined data indicated that the BNA-mAbs blocked virus replication in vitro by inhibiting NA activity and suggested that mAbs directly targeting the NA enzymatic active site had potentially more potent virus neutralization capacities in vitro.

### Example 3: BNA-mAbs are Broadly Protective in a Lethal Murine Model of IBV Infection

Next, we tested the protective capacities of the BNA-mAbs in vivo using a lethal murine model of IBV infection. The mAbs were tested in both prophylactic and therapeutic settings against IBVs that were isolated at the Mount Sinai Medical Center and were representative of currently circulating IBVs. All BNA-mAbs conferred robust protection (100% survival) against B/New York/PV00094/17 (Y) when 5 mg/kg was injected intraperitoneally 2 h before intranasal virus challenge (FIG. 3, A and B). Remarkably, robust protection was maintained even when the mAb dose was reduced to 1 mg/kg (FIG. 3.1, E and F). Lung viral load was assessed at 3 and 6 days post-challenge. Mean lung titers trended lower in all groups treated with BNA-mAbs compared to the control-treated group by 3 days post-infection, with the 1G05-treated animals showing an almost two-log decrease in viral load (FIG. 3, C). By 6 days post-infection, viral replication was markedly lower or undetectable in all anti-NA treated animals compared to those injected with the negative control mAb (FIG. 3, D). Robust prophylactic protective capacity of the BNA-mAbs also was observed when animals were challenged with a different IBV belonging to the B/Victoria/2/87-like lineage, B/New York/PV00081/18 (V) (FIG. 3, E and F).

We assessed the therapeutic potential of the BNA-mAbs by infecting mice with a lethal dose of B/New York/PV00094/17 (Y), and then treating with 5 mg/kg BNA-mAbs after 72 h. All animals experienced weight loss in this setting. Only 2E01 provided 100% protection against mortality (FIGS. 3, G and H), and 1G05 and 3C01 protected 80% of the mice against lethality (FIG. 3, G and H). These data indicated that the BNA-mAbs protected against lethal IBV infection in vivo and confirmed the superiority of some (1G05 and 2E01) anti-NA mAbs that potentially targeted the NA enzymatic active site in affording protection.

### Example 4: Overall Structure of Influenza B Virus NA-1G05 and NA-2E01 Complexes

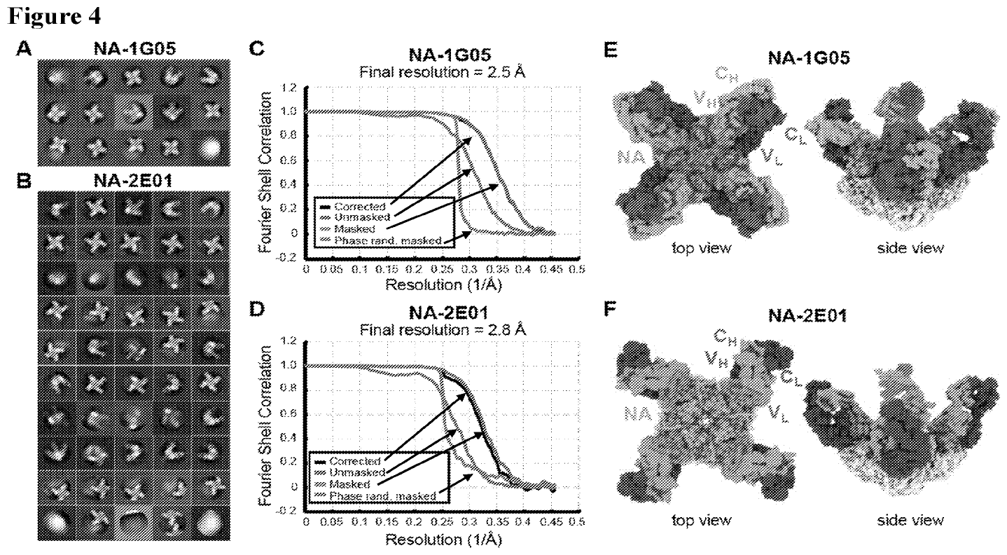

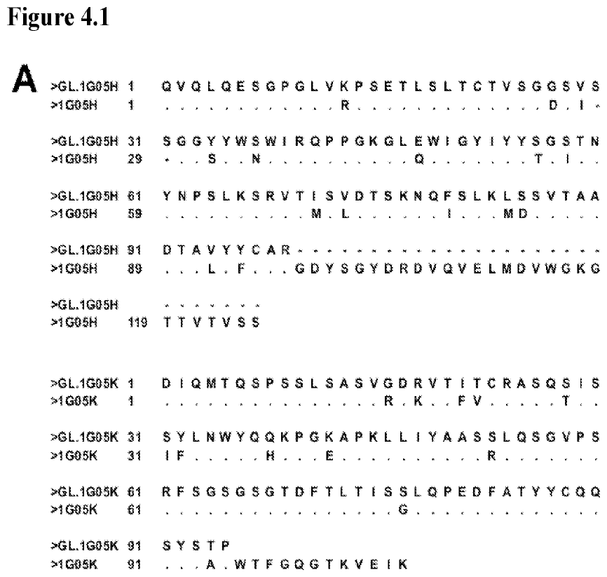

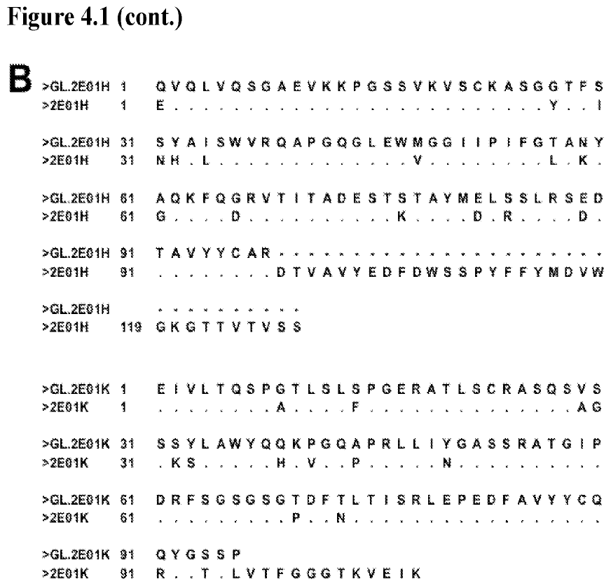

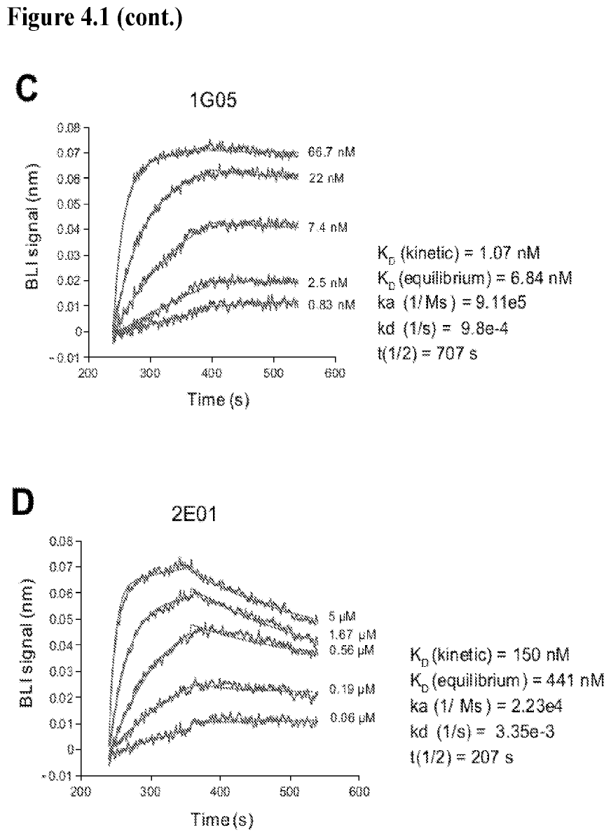

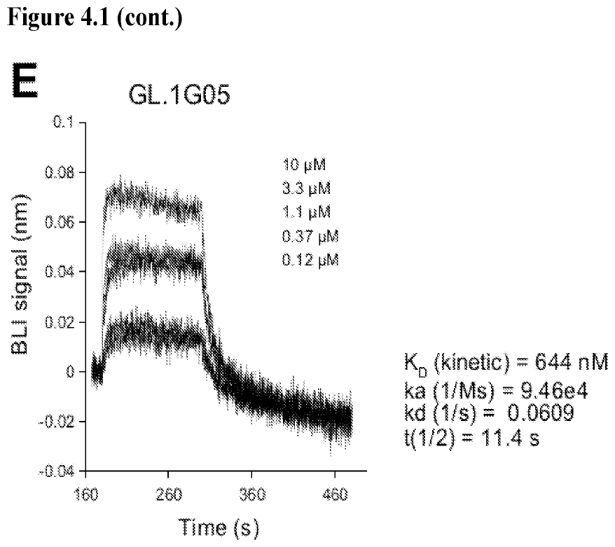

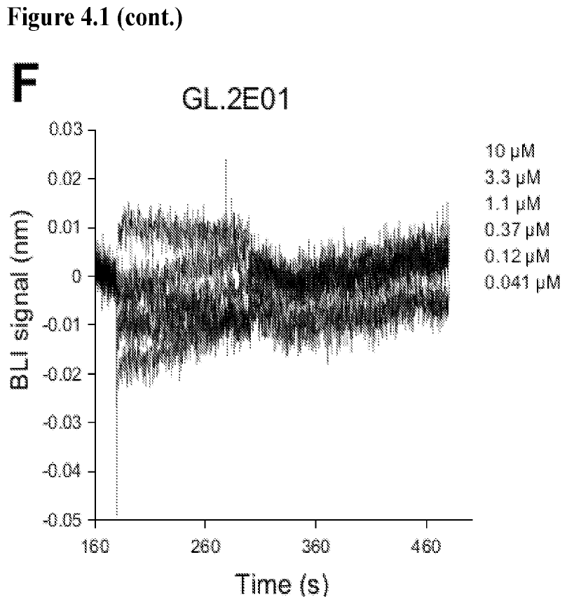

Both 1G05 and 2E01 inhibited the enzymatic activity of NAs from a broad range of IBV strains and blocked IBV infection in vitro and in vivo. Both mAbs specifically bound to IBV NA and did not bind IVA NA (FIG. 3.1, G-I). Primary sequence alignments of 1G05 and 2E01 with their inferred germline ancestors showed that both had accumulated substantial mutations (FIG. 4.1, A and B). To determine how these mutations altered their binding to BNA, we expressed monomeric antigen-binding fragments (Fabs) of mAbs 1G05 and 2E01 and their corresponding inferred germline ancestors and performed biolayer interferometry (BLI) to measure their binding kinetics to B/Phuket/3073/2013 NA. The mAb 1G05 had higher binding affinity and longer half-life (t_{1/2}) than 2E01 (FIG. 4.1, C and D). The inferred germline ancestor of 1G05 had substantially lower NA binding, whereas that of 2E01 displayed no detectable NA binding (FIG. 4.1, E and F). We performed single particle cryo-electron microscopy (cryo-EM) to investigate the epitopes targeted by these mAbs by solving their Fab structures in a complex with B/Phuket/3073/2013 NA, designated as NA-1G05 and NA-2E01 (Table 11). For both datasets, 2D classification showed that particle orientations on grids were well-distributed (FIG. 4, A and B). The 3D reconstruction of both NA-Fab complexes identified tetrameric NA decorated by one Fab per NA promoter (FIG. 4, C and D). The final maps were interpreted at 2.5 Å and 2.8 Å resolution for NA-1G05 and NA-2E01, respectively (FIG. 4, E and F).

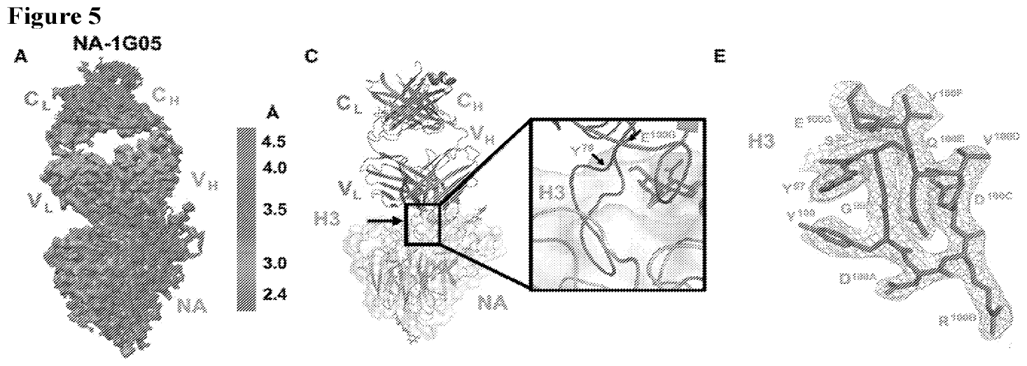

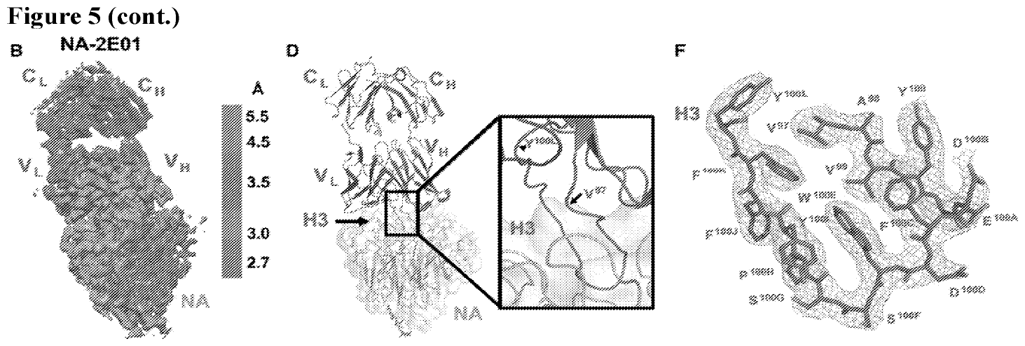

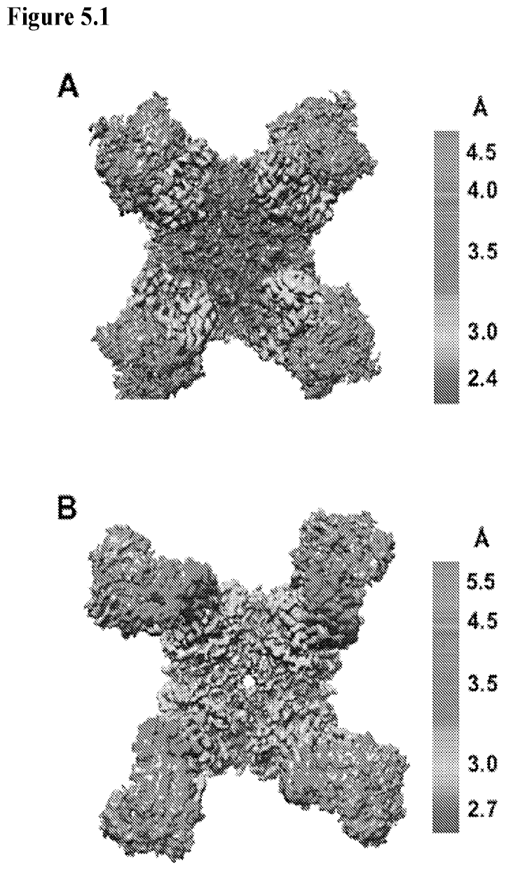

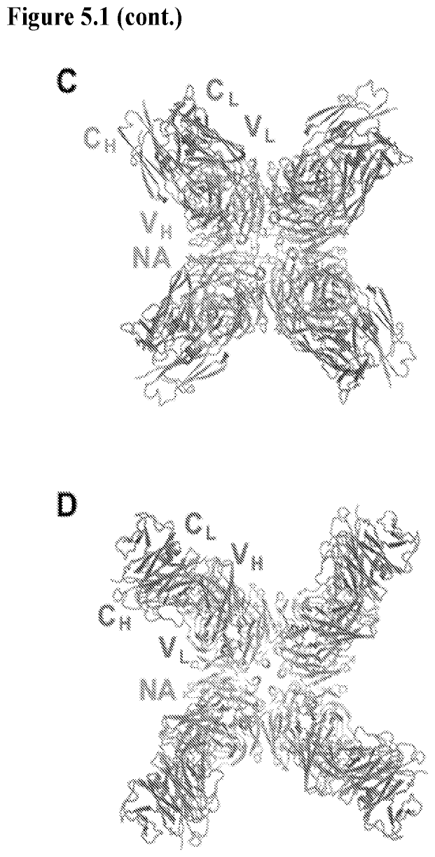

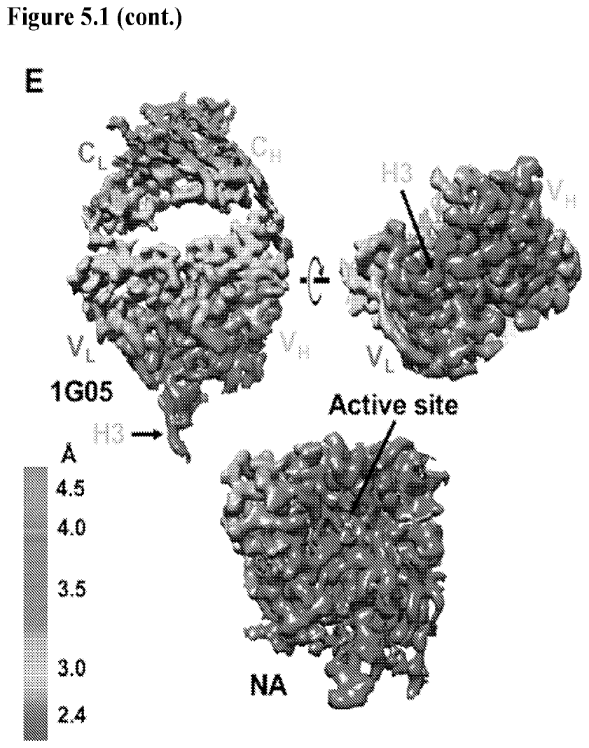

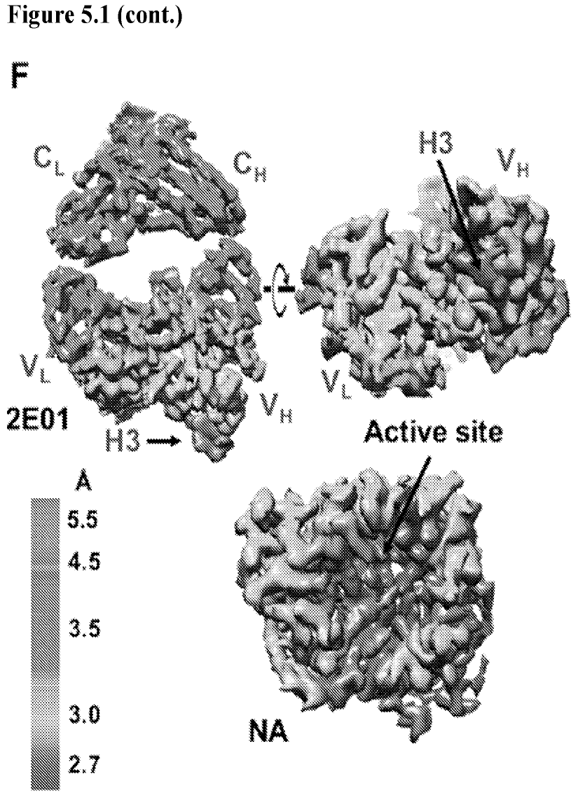

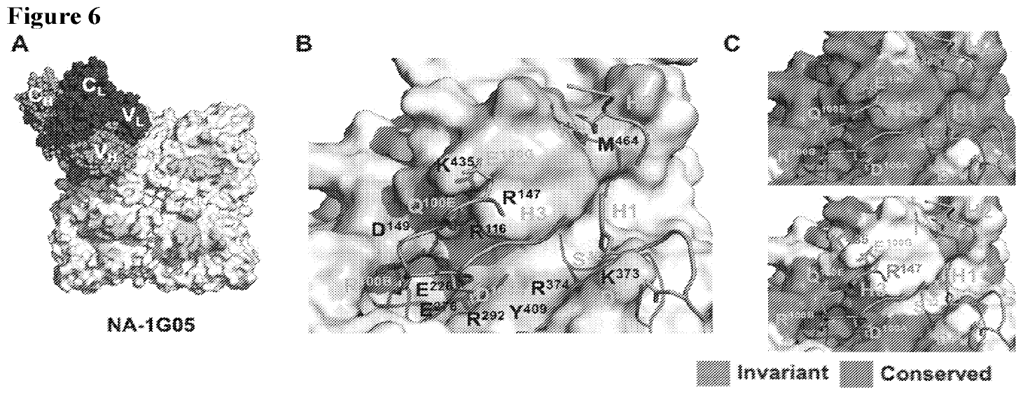

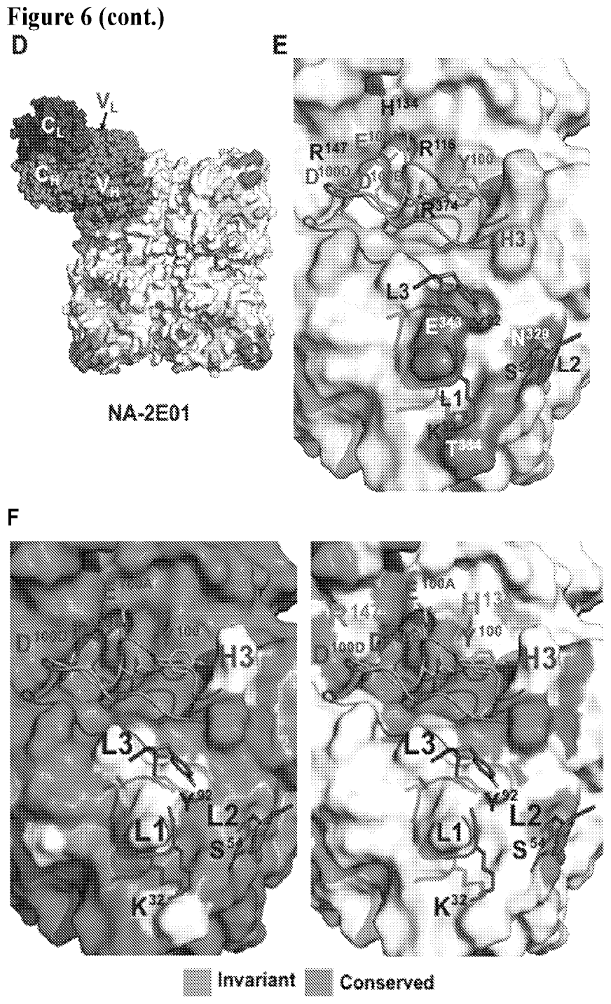

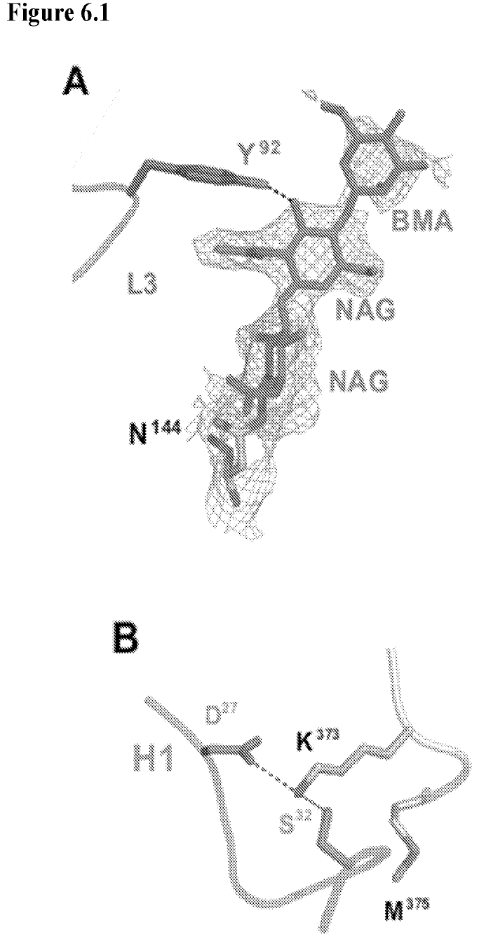

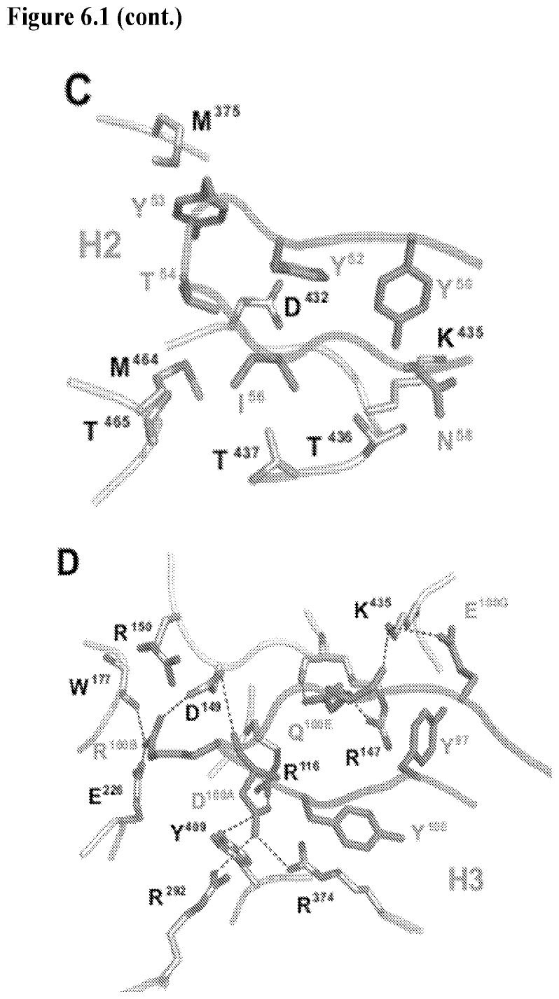

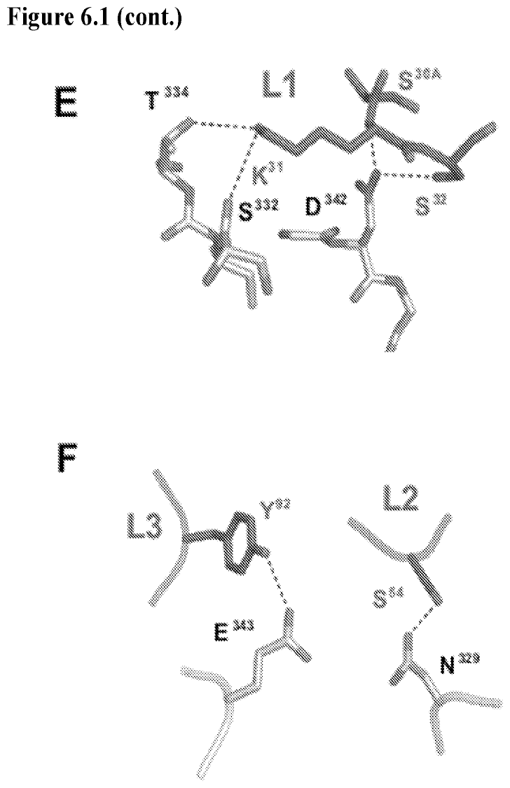

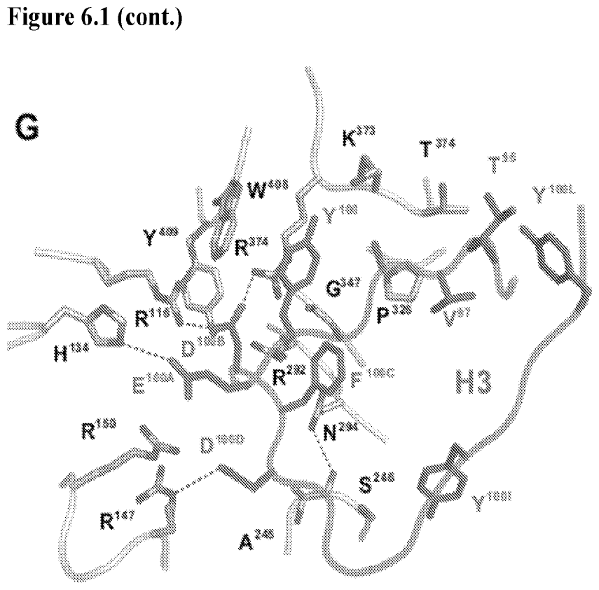

Local resolution analyses of the final reconstructed electron density maps revealed that the resolution decreased from the NA core to the constant domains of each Fab, suggesting flexibility of the Fab elbow regions (FIG. 5, A and B; FIG. 5.1, A, B, E, and F). The NA in NA-1G05 was highly ordered and a complete atomic model was built (FIG. 5.1, C). Four NA regions in the NA-2E01 complex were not well ordered, including residues E¹⁰⁵-S¹¹⁰ and G¹⁴⁰-Y¹⁴³ in the 150-loop, G⁴³³-T⁴³⁷ in the 430-loop, and W⁴⁵⁶-L⁴⁶⁶ at the C terminus (data not shown), however the atomic model for the remaining part of the NA-2E01 tetramer was built properly (FIG. 5.1, D). The variable domains of both Fabs and the complementarity-determining regions (CDRs) were very well resolved (FIG. 5, C-F) and provided critical information for epitope analysis. The buried surface area of the NA and 1G05 interface was ˜1100 A° 2, with the heavy chain (HC) accounting for ˜90% of the interaction. The 1G05-HC dominated the Fab binding to NA by CDR-H3 protruding into the active pocket of NA (FIG. 5, C), whereas the light chain (LC) only contributed to the interface by interacting with one N-acetylglucosamine moiety attached to residue N144 on NA (FIG. 6.1, A). In NA-2E01, the buried surface area was ˜960 A° 2, with the LC accounting for more surface area than 1G05, representing ˜20% of the total interaction (200 A° 2 of the total 960 A° 2 interface). The 2E01-HC displayed approximately 41.6° counter-clockwise rotation compared to 1G05-HC binding to NA (FIG. 6, A and D). Similar to binding of 1G05, the CDR-H3 of 2E01 also played a major role in engaging the NA active site (FIG. 5, D). These data established that both 1G05 and 2E01 directly targeted the IBV NA active site, consistent with their NI activity.

TABLE 11
Cryo-EM data collection and refinement statistics

| | NA-1G05 | NA-2E01 |
|---|---|---|
| | (EMD-21042, PDB ID | (EMD-21043, PDB ID |
| | 6V4N) | 6V4O) |
| Data collection and processing | | |
| Magnification, x | 105,000 (K2) | 105,000 (K2) |
| Voltage, keV | 300 | 300 |
| Electron exposure, e⁻/Å² | 66 (K2) | 66 (K2) |
| Pixel size, Å | 1.1 | 1.1 |
| Symmetry imposed | C4 | C4 |
| Model resolution, Å | 2.5 (0.143) | 2.8 (0.143) |
| (FSC threshold) | | |
| Map sharpening B factor, Å² | −30 | −30 |
| Refinement | | |
| Protein residues | 3287 | 3204 |
| Ligands | 8 (BMA), 24 (NAG) 4 (Ca2+) | 4 (BMA), 8 (NAG) 4 (Ca2+) |
| Solvent | 182 | 0 |
| RMSD* | | |
| RMS (bonds) (Å) | 0.007 | 0.019 |
| RMS(angles) (°) | 0.716 | 1.188 |
| Average B-factor (Å²) | 68.96 | 89.41 |
| Ramachandran plot^{a} (residues, %) | | |
| Favored | 93.52 | 90.12 |
| Allowed | 6.48 | 9.88 |
| Outliers | 0.00 | 0 |
| Rotamer outliers (%) | 0.89 | 2.88 |
| Clashscore | 3.63 | 7.43 |
| MolProbity score | 1.71 | 2.30 |

### Example 5: Defining NA Epitope Residues Responsible for IBV Specificity of 1G05 and 2E01

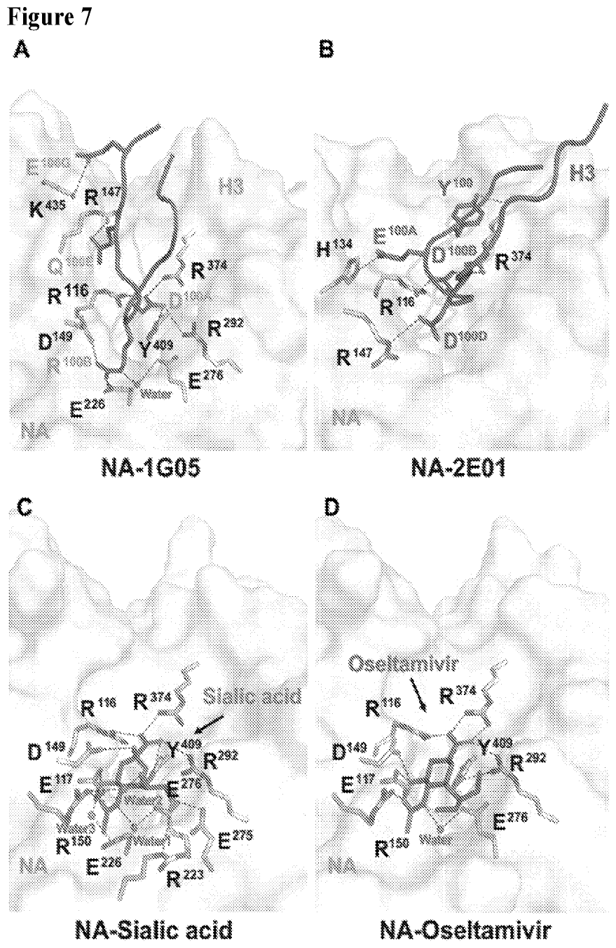

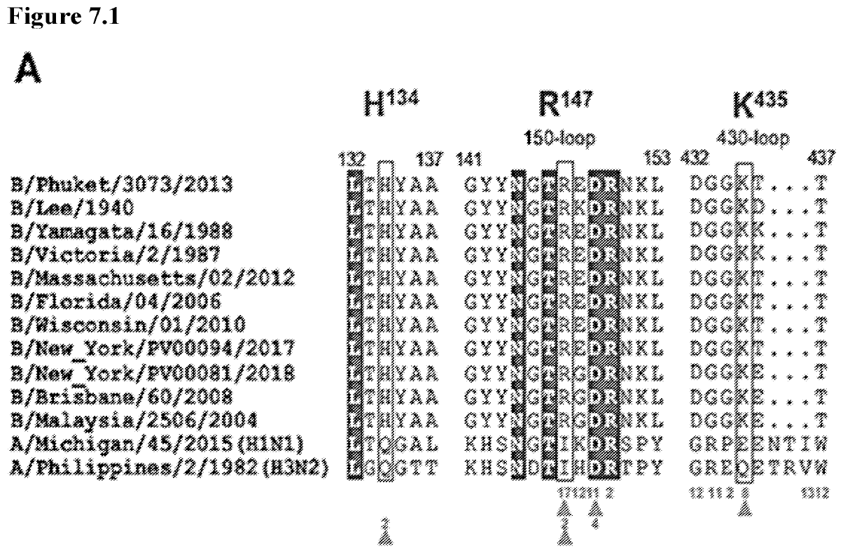

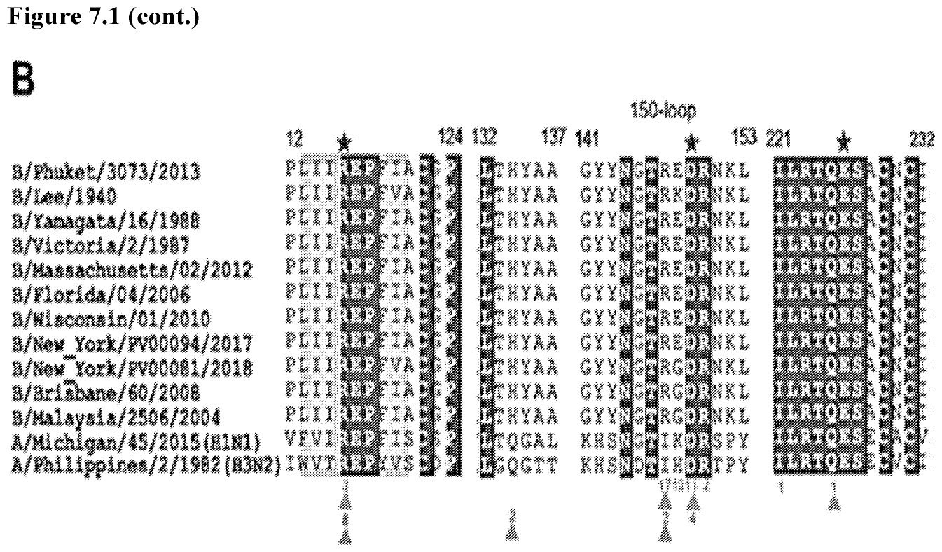

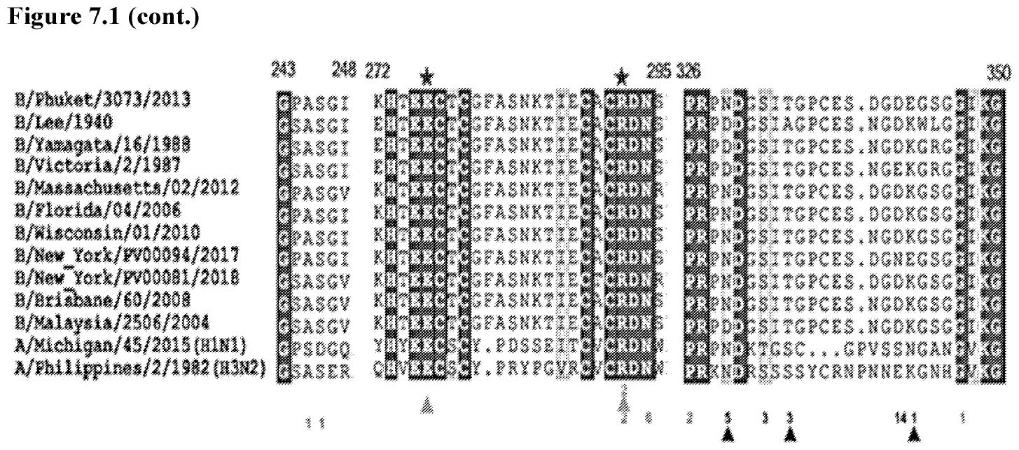

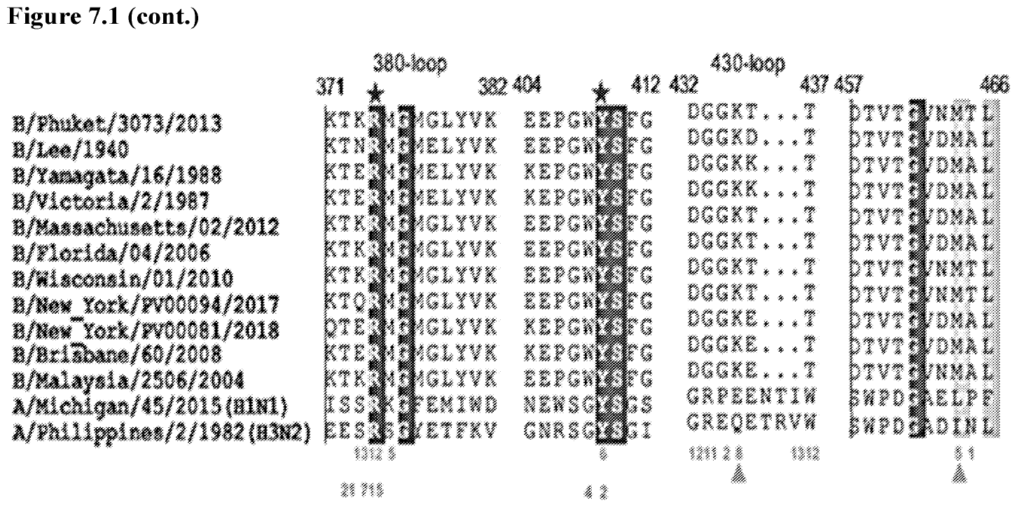

Next, we examined the structural epitopes engaged by 1G05 and 2E01 (FIG. 6). CDR-H3 dominated the contact interface for 1G05, but H1 and H2 also contributed to NA binding (FIG. 6, B and FIG. 6.1, B-D). We investigated the mechanism determining the strain specificities of 1G05 and 2E01 by analyzing conservation of residues within each epitope among NA sequences for multiple influenza virus strains. The 1G05 epitope residues were nearly invariant among IBV strains for which NA activity was inhibited (FIG. 6, C, upper panel). By contrast, epitope conservation analysis indicated that two key IBV NA residues engaged by 1G05 CDR-H3 (R¹⁴⁷ and K⁴³⁵) were not conserved among IAV strains, for which no inhibition was observed (FIG. 6, C, lower panel). In IAV strains, the equivalent residue of IBV NA R¹⁴⁷ is an isoleucine, whereas for K⁴³⁵ it is either a glutamic acid or a glutamine (FIG. 7.1, A). Thus, R¹⁴⁷ and K⁴³⁵ may contribute to the IBV strain specificity of 1G05. The mAb 2E01 engaged NA primarily using CDR-H3 and all three LC CDRs (FIG. 6, E and FIG. 6.1, E-G). Epitope conservation analysis indicated that all of the 2E01 CDR-H3 contacts were invariant among IBV strains, whereas 2E01-LC contacts exhibited considerable IBV strain sequence variation (FIG. 6, F, left panel), indicating a dominant role for 2E01-HC in BNA epitope contacts. Although most of the NA epitope residues engaged by 2E01 CDR-H3 were conserved in IAV strains, two variable residues (H¹³⁴ and R¹⁴⁷) may function to determine the specificity of 2E01 (FIG. 6, F, right panel, and FIG. 7.1, A).

### Example 6: The CDR-H3 Loop from Both 1G05 and 2E01 Imitates Sialic Acid and Oseltamivir Binding to NA

In NA-1G05, the CDR-H3 had the most important role in binding by occupying the NA active site with residues D^{100A} and R^{100B}, which interacted with positively and negatively charged “patches” located at either end of the active site (FIG. 7, A). D^{100A} engaged a three-arginine cluster formed by NA residues R¹¹⁶, R²⁹² and R³⁷⁴, and Y⁴⁰⁹. R^{100B} formed salt bridges with residues D¹⁴⁹ and E²²⁶. D^{100A} and R^{100B} participated in extensive van der Waals contacts with additional NA epitope residues (Table 12). D^{100A} and R^{100B} blocked the active site in a similar manner as that observed for occupation of the active pocket by sialic acid and oseltamivir, and their carboxyl groups also were stabilized by the three-arginine cluster (FIG. 7, C and D). The primary amine group of oseltamivir (stabilized by D¹⁴⁹) combined with the acetamide group (stabilized by a water molecule and E²⁷⁶) shared a very similar binding mode to NA as 1G05 R^{100B}. NA residues contacting D^{100A} and R^{100B} from 1G05 CDR-H3 are considered important catalytic residues (Burmeister et al., 1993; Chong et al., 1992; Lentz et al., 1987; Taylor and von Itzstein, 1994) and are highly conserved in IAV and IBV NAs (FIG. 7.1, B). Residue Q^{100E} from CDR-H3 was stabilized by NA R¹⁴⁷, and residue E^{100G} was stabilized by NA K⁴³⁵ (FIG. 7, A). These results explained why 1G05 served as a strong NA inhibitor.

The 1G05 CDR-H3 interacted with both basic and acidic “patches” of the NA active pocket. By contrast, the 2E01 CDR-H3 primarily made polar interactions with basic residues on NA using D^{100B} and D^{100D} (FIG. 7, B). D^{100B} was stabilized by two conserved catalytic residues (R¹¹⁶ and R³⁷⁴), similar to D^{100A} in 1G05 CDR-H3, and mimicked the binding mode of the sialic acid and oseltamivir carboxylate to NA (FIG. 7, C and D). D^{100D} formed a salt bridge with R¹⁴⁷ (FIG. 7, B). The NA R¹⁵⁰ in NA-1G05 and NA-2E01 was not involved in ionic interactions as it was in the NA-sialic acid and NA-oseltamivir complexes, but it interacted with R^{100B} in 1G05 and E^{100A} in 2E01 through van der Waals contacts (FIGS. 6.1, D and G; Tables 12 and 13). Similarly, in the NA-2E01 complex structure, catalytically crucial residues NA R²⁹² and Y⁴⁰⁹ interacted with F^{100C} and D^{100B} in CDR-H3 of 2E01 via van der Waals contacts (FIG. 6.1, G and Table 13). In summary, both mAbs inhibited NA enzymatic activity by blocking the active pocket with long CDR-H3 loops and bound NA using similar mechanisms as those of the NA substrate sialic acid and NA inhibitor oseltamivir. The CDR-H3 of 1G05 protruded deeper into the active pocket than 2E01 and formed a more extensive polar interaction network with NA, explaining its higher binding affinity, longer t_{1/2}, and stronger inhibition of IBV NAs.

TABLE 12
Summary of van der Waals contacts across the NA-1G05 interface:

| | | | Number of |
|---|---|---|---|
| | | | Van der Waals |
| NA | IG05 | 1G05 | contacts |
| R¹¹⁶ | H^{D100A} (3) | H^{D27} | 6 |
| R¹⁴⁷ | H^{Q100E} (12), H^{Y97} (5) | H^{A28} | 1 |
| E¹⁴⁸ | H^{Q100E} (12) | H^{G30} | 3 |
| D¹⁴⁹ | H^{D100A} (4), H^{R100B} (7) | H^{S32} | 3 |
| R¹⁵⁰ | H^{R100B} (H, 2) | H^{Y50} | 3 |
| W¹⁷⁷ | H^{R100B} (H, 3) | H^{Y52} | 10 |
| S¹⁷⁸ | H^{R100B} (H, 1) | H^{Y53} | 1 |
| I²²¹ | H^{R100B} (H, 1) | H^{T54} | 10 |
| E²²⁶ | H^{R100B} (H, 1) | H^{I56} | 25 |
| R²⁹² | H^{D100A} (2) | H^{N58} | 7 |
| K³⁷³ | H^{G30} (3) H^{D27} (6) | H^{Y100} | 4 |
| | H^{A28} (1) H^{S32} (3) | | |
| R³⁷⁴ | H^{Y100} (4) H^{D100A} (8) | H^{D100A} | 23 |
| M³⁷⁵ | H^{G31} (4) H^{Y53} (1) | H^{R100B} | 15 |
| Y⁴⁰⁹ | H^{D100A} (6) | H^{Q100E} | 26 |
| D⁴³² | H^{Y52} (8) H^{T54} (4) | H^{E100G} | 6 |
| G⁴³³ | H^{T54} (1) H^{I56} (10) | L^{F32} | 1 |
| G⁴³⁴ | H^{Y52} (2) | L^{Y92} | 13 |
| K⁴³⁵ | H^{Q100E} (2) H^{E100G} (6) | | |
| T⁴³⁶ | H^{Y50} (3) H^{N58} (7) | | |
| | H^{I56} (3) | | |
| T⁴³⁷ | H^{I56} (12) | | |
| M⁴⁶⁴ | H^{T54} (H, 5) | | |
| T⁴⁶⁵ | H^{G55} (H, 1) | | |
| NAG⁷⁰¹ | L^{F32} (1) L^{Y92} (13) | | |

Van der Waals contacts summary

| | CDR-H1 | CDR-H2 | CDR-H3 | Total | CDR-L1 | | CDR-L3 | Total |
|---|---|---|---|---|---|---|---|---|
| | 27-32 | 50-58 | 100-100G | VH | 32 | CDRL2 | 92 | VL |
| NA | 17 | 57 | 79 | 153 | 1 | 0 | 13 | 14 |

TABLE 13
Summary of Van der Waals Contacts across the NA-2E01 interface

| | | | Number of |
|---|---|---|---|
| | | | Van der Waals |
| NA | 2E01 | 2E01 | contacts |
| R¹¹⁶ | H^{D100B} (8) | H^{H32} | 4 |
| H¹³⁴ | H^{E100A} (2) | H^{T96} | 13 |
| R¹⁴⁷ | H^{D100D} (2) | H^{V97} | 7 |
| R¹⁵⁰ | P^{E100A} (4) | H^{A98} | 3 |
| A²⁴⁵ | H^{D100D}(1) | H^{Y100} | 14 |
| S²⁴⁶ | H^{Y100I} (1) | H^{E100A} | 6 |
| R²⁹² | H^{F100C} (2) | H^{D100B} | 16 |
| N²⁹⁴ | H^{F100C} (6) | H^{F100C} | 9 |
| P³²⁶ | H^{V97} (2) | H^{D100D} | 3 |
| G³⁴⁷ | H^{F100C} (1) | H^{Y100I} | 1 |
| T³⁷² | H^{T96} (13) H^{V97} (4) | H^{Y100L} | 3 |
| | H^{A98} (1) H^{Y100L} (3) | | |
| K³⁷³ | H^{H32} (4) H^{A98} (2) | L^{S30A} | 3 |
| | H^{Y100} (1) | | |
| R³⁷⁴ | H^{Y100} (9) H^{D100B} (6) | L^{K31} | 11 |
| W⁴⁰⁸ | H^{Y100} (4) | L^{S32} | 6 |
| Y⁴⁰⁹ | H^{D100B} (2) | L^{S53} | 5 |
| N³²⁹ | L^{S53} (5) | | |
| S³³² | L^{K31} (3) | | |
| T³³⁴ | L^{K31} (3) | | |
| D³⁴² | L^{S30A} (3) L^{K31} (5) | | |
| | L^{S32} (6) | | |
| E³⁴³ | L^{Y91} (1) | | |

Van der Waals contacts summary

| | CDR-H1 | CDR-H2 | CDR-H3 | Total | CDR-L1 | CDR-L2 | CDR-L3 | Total |
|---|---|---|---|---|---|---|---|---|
| | 25-32 | 50-57 | 96-100L | VH | 25-33 | 49-54 | 90-100 | VL |
| NA | 4 | 0 | 75 | 79 | 9 | 5 | 1 | 15 |

### Example 7: Discussion of Examples 1 to 6

Studies of antibody-mediated immunity to influenza viruses traditionally focus on HA as a target (Ellebedy and Ahmed, 2012; Wilson and Andrews, 2012). However, anti-NA antibodies have an important role in providing a comprehensive immune-mediated protection against influenza virus infection (Krammer et al., 2018). In this study, we described the isolation and functional characterization of seven novel human BNA-mAbs derived from an infected patient. All mAbs protected mice in a lethal IBV challenge model using clinically isolated IBV strains that belonged to the two antigenically distinct IBV lineages. We determined the structural basis for broad NA inhibition exhibited by two mAbs that displayed the most potent reactivity in vitro and in vivo. The described BNA-mAbs are potentially valuable as therapeutics due to the limitations of currently approved antiviral drugs targeting influenza. In vitro studies show that IBVs are less susceptible than IAVs to FDA-approved NA inhibitors and cap-dependent endonuclease inhibitors, thereby complicating treatment of IBV infections (Burnham et al., 2013; Mishin et al., 2019). This is especially true in the pediatric population where oseltamivir is less effective than zanamivir, with the latter approved only for children aged R7 years old. Three of the isolated mAbs (2E01, 1G05, and 3C01) protected 80%-100% of mice from mortality in stringent challenge models with recent IBV isolates, even when animals were treated 72 h after virus challenge. These data clearly demonstrated the potential of the identified BNA-mAbs for use as human therapeutics.

Seasonal epidemics caused by IBVs are responsible for up to 52% of influenza-associated pediatric mortality during the last fifteen years (Burnham et al., 2013; Govorkova and McCullers, 2013). Many aspects of immunity to IBVs are understudied compared to those of IAVs, particularly antibody-mediated immunity to IBV NA. Panels of murine and human mAbs specific for IBV NA have been reported (Piepenbrink et al., 2019; Wohlbold et al., 2017), although comprehensive functional and structural analyses of how these mAbs inhibit NA or mediate protection are lacking. Anti-NA antibodies provide in vivo protection by blocking either viral transport through the mucosal layer lining the lung epithelium or viral egress from infected cells (Eichelberger et al., 2018). Here, we demonstrated that all seven of the isolated BNA-mAbs displayed NA inhibitory (NAI) activity, virus neutralization capacity, and ability to mediate secondary effector functions as evidenced by their activity in an ADCC bioreporter assay. Our detailed structural analyses indicated that the NAI activities of the most potent mAbs were mediated by a sialic acid and oseltamivir-like mode of binding to residues within the NA enzymatic active site. The in vivo protection exhibited by these mAbs was likely mediated by a combination of these mechanisms.

Structurally mapped human anti-IAV NA antibodies are predominantly subtype specific (Gilchuk et al., 2019; Zhu et al., 2019). Our group isolated 1G01, a broadly protective human anti-NA mAb that predominantly targets IAVs (Stadlbauer et al., 2019). Similar to 1G05 and 2E01, 1G01 CDR-H3 accounts for the majority of NA recognition. However, CDR-H3 loops in 1G05 and 2E01 protrude into the NA active pocket from an entirely different angle than those of 1G01. The epitope recognized by both 1G05 and 2E01 is within one protomer, whereas in NA-1G01, the side chain of Y⁹⁷ from CDR-L3 is stabilized by hydrophobic interaction with W^{456¢} from an adjacent NA protomer. The NA-1G01 catalytic arginines (R¹¹⁸ and R³⁷¹) are engaged with a backbone carbonyl of R^{100C} in CDR-H3, whereas the corresponding arginines (R¹¹⁶ and R³⁷⁴) in NA-1G05 and NA-2E01 are stabilized by side chains of D^{100A} and D^{100B}, respectively. These combined results indicate that the epitopes recognized by 1G05 and 2E01 are significantly different from those of 1G01, despite the high similarity between NA active sites in IBVs and IAVs. Similar to 1G01, 1G05 and 2E01 contain a longer than average HCDR3, which may be an important feature of mAbs that efficiently block the NA enzymatic active site. However, this feature is not sufficient to block the active site; mAb 1D05 contains the longest HCDR3 among the seven mAbs (25 aa), but did not display strong or broad NA inhibition activity.

Previously reported atomic structures of mAb/IVB NA complexes used murine mAbs targeting non-active site epitopes, which were solved at ˜25 A° resolution (Wohlbold et al., 2017). A study reported a panel of broadly protective anti-BNA human mAbs from vaccinated individuals (Piepenbrink et al., 2019). Although overall mutation levels in the present study were similar with that previous report, there was no overlap among the IGHV or IGLV genes or obvious similarity in the CDR3 regions of the mAbs. Notably, 1G05 and 2E01 inhibited the NA enzymatic activity of a variety of IBVs, ranging from one of the earliest IBV isolates (B/Lee/1940) to contemporary isolates (B/New York/PVI/81/2018), spanning more than 75 years of antigenic drift. Epitope conservation analysis showed that key NA residues of the epitopes targeted by these two mAbs were highly conserved. Structure-based sequence alignment showed that these residues were conserved among the IBV strains circulating during the 2019/20 influenza season that caused the most recent IBV outbreak.

### REFERENCES

1. Adams, P. D., Afonine, P. V., Bunko'czi, G., Chen, V. B., Davis, I. W., Echols, N., Headd, J. J., Hung, L.-W., Kapral, G. J., Grosse-Kunstleve, R. W., et al. (2010). PHENIX: a comprehensive Python-based system for macromolecular structure solution. Acta Crystallogr. D Biol. Crystallogr. 66, 213-221.
2. Brochet, X., Lefranc, M.-P., and Giudicelli, V. (2008). IMGT/V-QUEST: the highly customized and integrated system for IG and TR standardized V-J and V-D-J sequence analysis. Nucleic Acids Res. 36, W503-W508.
3. Burmeister, W. P., Henrissat, B., Bosso, C., Cusack, S., and Ruigrok, R. W. (1993). Influenza B virus neuraminidase can synthesize its own inhibitor. Structure 1, 19-26.
4. Burnham, A. J., Baranovich, T., and Govorkova, E. A. (2013). Neuraminidase inhibitors for influenza B virus infection: efficacy and resistance. Antiviral Res. 100, 520-534.
5. Chen, V. B., Arendall, W. B., 3rd, Headd, J. J., Keedy, D. A., Immormino, R. M., Kapral, G. J., Murray, L. W., Richardson, J. S., and Richardson, D. C. (2010). MolProbity: all-atom structure validation for macromolecular crystallography. Acta Crystallogr. D Biol. Crystallogr. 66, 12-21.
6. Chen, S., McMullan, G., Faruqi, A. R., Murshudov, G. N., Short, J. M., Scheres, S. H. W., and Henderson, R. (2013). High-resolution noise substitution to measure overfitting and validate resolution in 3D structure determination by single particle electron cryomicroscopy. Ultramicroscopy 135, 24-35.
7. Chen, Y.-Q., Wohlbold, T. J., Zheng, N.-Y., Huang, M., Huang, Y., Neu, K. E., Lee, J., Wan, H., Rojas, K. T., Kirkpatrick, E., et al. (2018). Influenza Infection in Humans Induces Broadly Cross-Reactive and Protective Neuraminidase-Reactive Antibodies. Cell 173, 417-429.e10.
8. Chong, A. K., Pegg, M. S., Taylor, N. R., and von Itzstein, M. (1992). Evidence for a sialosyl cation transition-state complex in the reaction of sialidase from influenza virus. Eur. J. Biochem. 207, 335-343.
9. Crowe, J. E., Jr. (2017). Principles of broad and potent antiviral human anti-bodies: insights for vaccine design. Cell Host Microbe 22, 193-206.
10. DiLillo, D. J., Tan, G. S., Palese, P., and Ravetch, J. V. (2014). Broadly neutralizing hemagglutinin stalk-specific antibodies require FcgR interactions for protection against influenza virus in vivo. Nat. Med. 20, 143-151.
11. Eichelberger, M. C., Morens, D. M., and Taubenberger, J. K. (2018). Neuraminidase as an influenza vaccine antigen: a low hanging fruit, ready for picking to improve vaccine effectiveness. Curr. Opin. Immunol. 53, 38-44.
12. Ellebedy, A. H., and Ahmed, R. (2012). Re-engaging cross-reactive memory B cells: the influenza puzzle. Front. Immunol. 3, 53.
13. Ellebedy, A. H., and Webby, R. J. (2009). Influenza vaccines. Vaccine 27 (Suppl 4), D65-D68.
14. Ellebedy, A. H., Jackson, K. J. L., Kissick, H. T., Nakaya, H. I., Davis, C. W., Roskin, K. M., McElroy, A. K., Oshansky, C. M., Elbein, R., Thomas, S., et al. (2016). Defining antigen-specific plasmablast and memory B cell subsets in human blood after viral infection or vaccination. Nat. Immunol. 17, 1226-1234.
15. Ellebedy, A. H., Nachbagauer, R., Jackson, K. J. L., Dai, Y.-N., Han, J., Alsoussi, W. B., Davis, C. W., Stadlbauer, D., Rouphael, N., Chromikova, V., et al. (2020). Adjuvanted H5N1 influenza vaccine enhances both cross-reactive memory B cell and strain-specific naive B cell responses in humans. Proc. Natl. Acad. Sci. USA 117, 17957-17964.
16. Emsley, P., and Cowtan, K. (2004). Coot: model-building tools for molecular graphics. Acta Crystallogr. D Biol. Crystallogr. 60, 2126-2132.
17. Erbelding, E. J., Post, D. J., Stemmy, E. J., Roberts, P. C., Augustine, A. D., Ferguson, S., Paules, C. I., Graham, B. S., and Fauci, A. S. (2018). A universal influenza vaccine: the strategic plan for the national institute of allergy and infectious diseases. J. Infect. Dis. 218, 347-354.
18. Gilchuk, I. M., Bangaru, S., Gilchuk, P., Irving, R. P., Kose, N., Bombardi, R. G., Thornburg, N.J., Creech, C. B., Edwards, K. M., Li, S., et al. (2019). Influenza H7N9 Virus Neuraminidase-Specific Human Monoclonal Antibodies Inhibit Viral Egress and Protect from Lethal Influenza Infection in Mice. Cell Host Microbe 26, 715-728.e8.
19. Giudicelli, V., Brochet, X., and Lefranc, M.-P. (2011). IMGT/V-QUEST: IMGT standardized analysis of the immunoglobulin (IG) and T cell receptor (TR) nucleotide sequences. Cold Spring Harb. Protoc. 2011, 695-715.
20. Goddard, T. D., Huang, C. C., Meng, E. C., Pettersen, E. F., Couch, G. S., Morris, J. H., and Ferrin, T. E. (2018). UCSF ChimeraX: Meeting modern challenges in visualization and analysis. Protein Sci. 27, 14-25.
21. Govorkova, E. A., and McCullers, J. A. (2013). Therapeutics against influenza. Curr. Top. Microbiol. Immunol. 370, 273-300.
22. Gubareva, L. V., Matrosovich, M. N., Brenner, M. K., Bethell, R. C., and Webster,
23. R. G. (1998). Evidence for zanamivir resistance in an immunocompromised child infected with influenza B virus. J. Infect. Dis. 178, 1257-1262.
24. Hayward, S., and Berendsen, H. J. (1998). Systematic analysis of domain motions in proteins from conformational change: new results on citrate synthase and T4 lysozyme. Proteins 30, 144-154.
25. Ho, I. Y., Bunker, J. J., Erickson, S. A., Neu, K. E., Huang, M., Cortese, M., Pulendran, B., and Wilson, P. C. (2016). Refined protocol for generating monoclonal antibodies from single human and murine B cells. J. Immunol. Methods 438, 67-70.
26. Ison, M. G., Gubareva, L. V., Atmar, R. L., Treanor, J., and Hayden, F. G. (2006). Recovery of drug-resistant influenza virus from immunocompromised patients: a case series. J. Infect. Dis. 193, 760-764.
27. Kawai, N., Ikematsu, H., Iwaki, N., Maeda, T., Satoh, I., Hirotsu, N., and Kashiwagi, S. (2006). A comparison of the effectiveness of oseltamivir for the treatment of influenza A and influenza B: a Japanese multicenter study of the 2003-2004 and 2004-2005 influenza seasons. Clin. Infect. Dis. 43, 439-444.
28. Krammer, F. (2019). The human antibody response to influenza A virus infection and vaccination. Nat. Rev. Immunol. 19, 383-397.
29. Krammer, F., Fouchier, R. A. M., Eichelberger, M. C., Webby, R. J., Shaw-Saliba, K., Wan, H., Wilson, P. C., Compans, R. W., Skountzou, I., and Monto, A. S. (2018). NAction! How Can Neuraminidase-Based Immunity Contribute to Better Influenza Virus Vaccines? MBio 9, e02332-17.
30. Kucukelbir, A., Sigworth, F. J., and Tagare, H. D. (2014). Quantifying the local resolution of cryo-EM density maps. Nat. Methods 11, 63-65.
31. Langat, P., Raghwani, J., Dudas, G., Bowden, T. A., Edwards, S., Gall, A., Bedford, T., Rambaut, A., Daniels, R. S., Russell, C. A., et al. (2017). Genome-wide evolutionary dynamics of influenza B viruses on a global scale. PLoS Pathog. 13, e1006749.
32. Lentz, M. R., Webster, R. G., and Air, G. M. (1987). Site-directed mutation of the active site of influenza neuraminidase and implications for the catalytic mechanism. Biochemistry 26, 5351-5358.
33. Margine, I., Palese, P., and Krammer, F. (2013). Expression of functional re-
34. combinant hemagglutinin and neuraminidase proteins from the novel H7N9 influenza virus using the baculovirus expression system. J. Vis. Exp. 6, e51112.
35. McMahon, M., Kirkpatrick, E., Stadlbauer, D., Strohmeier, S., Bouvier, N. M., and Krammer, F. (2019). Mucosal Immunity against Neuraminidase Prevents Influenza B Virus Transmission in Guinea Pigs. MBio 10, e00560-19.
36. Mishin, V. P., Patel, M. C., Chesnokov, A., De La Cruz, J., Nguyen, H. T., Lollis, L., Hodges, E., Jang, Y., Barnes, J., Uyeki, T., et al. (2019). Susceptibility of influenza A, B, C, and D viruses to baloxavirl. Emerg. Infect. Dis. 25, 1969-1972.
37. Murin, C. D., Wilson, I. A., and Ward, A. B. (2019). Antibody responses to viral infections: a structural perspective across three different enveloped viruses. Nat. Microbiol. 4, 734-747.
38. Paul Glezen, W., Schmier, J. K., Kuehn, C. M., Ryan, K. J., and Oxford, J. (2013). The burden of influenza B: a structured literature review. Am. J. Public Health 103, e43-e51.
39. Paules, C. I., Marston, H. D., Eisinger, R. W., Baltimore, D., and Fauci, A. S. (2017). The pathway to a universal influenza vaccine. Immunity 47, 599-603.
40. Paules, C. I., Sullivan, S. G., Subbarao, K., and Fauci, A. S. (2018). Chasing Seasonal Influenza—The Need for a Universal Influenza Vaccine. N. Engl. J. Med. 378, 7-9.
41. Pettersen, E. F., Goddard, T. D., Huang, C. C., Couch, G. S., Greenblatt, D. M., Meng, E. C., and Ferrin, T. E. (2004). UCSF Chimera—a visualization system for exploratory research and analysis. J. Comput. Chem. 25, 1605-1612.
42. Piepenbrink, M. S., Nogales, A., Basu, M., Fucile, C. F., Liesveld, J. L., Keefer, M. C., Rosenberg, A. F., Martinez-Sobrido, L., and Kobie, J. J. (2019). Broad and Protective Influenza B Virus Neuraminidase Antibodies in Humans after Vaccination and their Clonal Persistence as Plasma Cells. MBio 10, e00066-19.
43. Rota, P. A., Wallis, T. R., Harmon, M. W., Rota, J. S., Kendal, A. P., and Nerome,
44. K. (1990). Cocirculation of two distinct evolutionary lineages of influenza type B virus since 1983. Virology 175, 59-68.
45. Sato, M., Saito, R., Sato, I., Tanabe, N., Shobugawa, Y., Sasaki, A., Li, D., Suzuki, Y., Sato, M., Sakai, T., et al. (2008). Effectiveness of oseltamivir treatmeat among children with influenza A or B virus infections during four successive winters in Niigata City, Japan. Tohoku J. Exp. Med. 214, 113-120.
46. Sievers, F., and Higgins, D. G. (2014). Clustal Omega, accurate alignment of very large numbers of sequences. Methods Mol. Biol. 1079, 105-116.
47. Smith, K., Garman, L., Wrammert, J., Zheng, N.-Y., Capra, J. D., Ahmed, R., and Wilson, P. C. (2009). Rapid generation of fully human monoclonal anti-bodies specific to a vaccinating antigen. Nat. Protoc. 4, 372-384.
48. Stadlbauer, D., Amanat, F., Strohmeier, S., Nachbagauer, R., and Krammer, F. (2018). Cross-reactive mouse monoclonal antibodies raised against the hemagglutinin of A/Shanghai/1/2013 (H7N9) protect against novel H7 virus isolates in the mouse model. Emerg. Microbes Infect. 7, 110.
49. Stadlbauer, D., Zhu, X., McMahon, M., Turner, J. S., Wohlbold, T. J., Schmitz, A. J., Strohmeier, S., Yu, W., Nachbagauer, R., Mudd, P. A., et al. (2019). Broadly protective human antibodies that target the active site of influenza virus neuraminidase. Science 366, 499-504.
50. Sugaya, N., Mitamura, K., Yamazaki, M., Tamura, D., Ichikawa, M., Kimura, K., Kawakami, C., Kiso, M., Ito, M., Hatakeyama, S., and Kawaoka, Y. (2007). Lower clinical effectiveness of oseltamivir against influenza B contrasted with influenza A infection in children. Clin. Infect. Dis. 44, 197-202.
51. Tamura, K., Stecher, G., Peterson, D., Filipski, A., and Kumar, S. (2013). MEGA6: Molecular Evolutionary Genetics Analysis version 6.0. Mol. Biol. Evol. 30, 2725-2729.
52. Tan, J., Asthagiri Arunkumar, G., and Krammer, F. (2018). Universal influenza virus vaccines and therapeutics: where do we stand with influenza B virus? Curr. Opin. Immunol. 53, 45-50.
53. Taylor, N. R., and von Itzstein, M. (1994). Molecular modeling studies on ligand binding to sialidase from influenza virus and the mechanism of catalysis. J. Med. Chem. 37, 616-624.
54. Virk, R. K., Jayakumar, J., Mendenhall, I. H., Moorthy, M., Lam, P., Linster, M., Lim, J., Lin, C., Oon, L. L. E., Lee, H. K., et al. (2020). Divergent evolutionarytrajectories of influenza B viruses underlie their contemporaneous epidemic activity. Proc. Natl. Acad. Sci. USA 117, 619-628.
55. Wagner, T., Merino, F., Stabrin, M., Moriya, T., Antoni, C., Apelbaum, A., Hagel, P., Sitsel, O., Raisch, T., Prumbaum, D., et al. (2019). SPHIRE-crYOLO is a fast and accurate fully automated particle picker for cryo-EM. Commun Biol 2, 218.
56. Wilson, P. C., and Andrews, S. F. (2012). Tools to therapeutically harness the human antibody response. Nat. Rev. Immunol. 12, 709-719.
57. Wohlbold, T. J., Nachbagauer, R., Xu, H., Tan, G. S., Hirsh, A., Brokstad, K. A., Cox, R. J., Palese, P., and Krammer, F. (2015). Vaccination with adjuvanted recombinant neuraminidase induces broad heterologous, but not heterosubtypic, cross-protection against influenza virus infection in mice. MBio 6, e02556.
58. Wohlbold, T. J., Podolsky, K. A., Chromikova, V., Kirkpatrick, E., Falconieri, V., Meade, P., Amanat, F., Tan, J., tenOever, B. R., Tan, G. S., et al. (2017). Broadly protective murine monoclonal antibodies against influenza B virus target highly conserved neuraminidase epitopes. Nat. Microbiol. 2, 1415-1424.
59. Wrammert, J., Koutsonanos, D., Li, G.-M., Edupuganti, S., Sui, J., Morrissey, M., McCausland, M., Skountzou, I., Hornig, M., Lipkin, W. I., et al. (2011). Broadly cross-reactive antibodies dominate the human B cell response against 2009 pandemic H1N1 influenza virus infection. J. Exp. Med. 208, 181-193.
60. Wu, N.C., and Wilson, I. A. (2018). Structural insights into the design of novel anti-influenza therapies. Nat. Struct. Mol. Biol. 25, 115-121.
61. Xu, X., Zhu, X., Dwek, R. A., Stevens, J., and Wilson, I. A. (2008). Structural characterization of the 1918 influenza virus H1N1 neuraminidase. J. Virol. 82, 10493-10501.
62. Zhang, K. (2016). Gctf: Real-time CTF determination and correction. J. Struct. Biol.
63. Zheng, S. Q., Palovcak, E., Armache, J.-P., Verba, K. A., Cheng, Y., and Agard, D. A. (2017). MotionCor2: anisotropic correction of beam-induced motion for improved cryo-electron microscopy. Nat. Methods 14, 331-332.
64. Zhu, X., Turner, H. L., Lang, S., McBride, R., Bangaru, S., Gilchuk, I. M., Yu, W., Paulson, J. C., Crowe, J. E., Jr., Ward, A. B., and Wilson, I. A. (2019). Structural Basis of Protection against H7N9 Influenza Virus by Human Anti-N9 Neuraminidase Antibodies. Cell Host Microbe 26, 729-738.e4.
65. Zivanov, J., Nakane, T., Forsberg, B. O., Kimanius, D., Hagen, W. J., Lindahl, E., and Scheres, S. H. (2018). New tools for automated high-resolution cryo-EM structure determination in RELION-3. eLife 7, e42166.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above methods, processes, and compositions without departing from the scope of the invention, it is intended that all matter contained in the above description and shown in the accompanying drawings shall be interpreted as illustrative and not in a limiting sense.

1.-2. (canceled)

3. An antibody or antigen-binding fragment, wherein the antibody or antigen-binding fragment comprises:
(a) an immunoglobulin heavy chain variable region comprising a CDR_{H1} having an amino acid sequence comprising any one of SEQ ID NOs: 1-7, a CDR_{H1} having an amino acid sequence comprising any one of SEQ ID NOs: 8-14, a CDR_{H2} having an amino acid sequence comprising any one of SEQ ID NOs: 15-21, or a combination thereof; and
(b) an immunoglobulin light chain variable region comprising a CDR_{L1} having an amino acid sequence comprising any one of SEQ ID NOs: 22-28, a CDR_{L2} having an amino acid sequence comprising any one of SEQ ID NOs: 29-34, a CDR_{L3} having an amino acid sequence comprising any one of SEQ ID NOs: 35-41, or a combination thereof.

4.-55. (canceled)

56. The antibody or antigen-binding fragment of claim 3, wherein the immunoglobulin heavy chain variable region comprises:
(a) a CDR_{H1} having an amino acid sequence comprising SEQ ID NO: 1, a CDR_{H2} having an amino acid sequence comprising SEQ ID NO: 8, and a CDR_{H3} having an amino acid sequence comprising SEQ ID NO: 15;
(b) a CDR_{H1} having an amino acid sequence comprising SEQ ID NO: 2, a CDR_{H2} having an amino acid sequence comprising SEQ ID NO: 9, and a CDR_{H3} having an amino acid sequence comprising SEQ ID NO: 16;
(c) a CDR_{H1} having an amino acid sequence comprising SEQ ID NO: 3, a CDR_{H2} having an amino acid sequence comprising SEQ ID NO: 10, and a CDR_{H3} having an amino acid sequence comprising SEQ ID NO: 17;
(d) a CDR_{H1} having an amino acid sequence comprising SEQ ID NO: 4, a CDR_{H2} having an amino acid sequence comprising SEQ ID NO: 11, and a CDR_{H3} having an amino acid sequence comprising SEQ ID NO: 18;
(e) a CDR_{H1} having an amino acid sequence comprising SEQ ID NO: 5, a CDR_{H2} having an amino acid sequence comprising SEQ ID NO: 12, and a CDR_{H3} having an amino acid sequence comprising SEQ ID NO: 19;
(f) a CDR_{H1} having an amino acid sequence comprising SEQ ID NO: 6, a CDR_{H2} having an amino acid sequence comprising SEQ ID NO: 13, and a CDR_{H3} having an amino acid sequence comprising SEQ ID NO: 20; or
(g) a CDR_{H1} having an amino acid sequence comprising SEQ ID NO: 7, a CDR_{H2} having an amino acid sequence comprising SEQ ID NO: 14, and a CDR_{H3} having an amino acid sequence comprising SEQ ID NO: 21.

57. The antibody or antigen-binding fragment of claim 3, wherein the immunoglobulin light chain variable region comprises:
(a) a CDR_{L1} having an amino acid sequence comprising SEQ ID NO: 22, a CDR_{L2} having an amino acid sequence comprising SEQ ID NO: 29, and a CDR_{L3} having an amino acid sequence comprising SEQ ID NO: 35;
(b) a CDR_{L1} having an amino acid sequence comprising SEQ ID NO: 23, a CDR_{L2} having an amino acid sequence comprising SEQ ID NO: 30, and a CDR_{L3} having an amino acid sequence comprising SEQ ID NO: 36;
(c) a CDR_{L1} having an amino acid sequence comprising SEQ ID NO: 24, a CDR_{L2} having an amino acid sequence comprising SEQ ID NO: 31, and a CDR_{L3} having an amino acid sequence comprising SEQ ID NO: 37;
(d) a CDR_{L1} having an amino acid sequence comprising SEQ ID NO: 25, a CDR_{L2} having an amino acid sequence comprising SEQ ID NO: 32, and a CDR_{L3} having an amino acid sequence comprising SEQ ID NO: 38;
(e) a CDR_{L1} having an amino acid sequence comprising SEQ ID NO: 26, a CDR_{L2} having an amino acid sequence comprising SEQ ID NO: 33, and a CDR_{L3} having an amino acid sequence comprising SEQ ID NO: 39;
(f) a CDR_{L1} having an amino acid sequence comprising SEQ ID NO: 27, a CDR_{L2} having an amino acid sequence comprising SEQ ID NO: 34, and a CDR_{L3} having an amino acid sequence comprising SEQ ID NO: 40; or
(g) a CDR_{L1} having an amino acid sequence comprising SEQ ID NO: 28, a CDR_{L2} having an amino acid sequence comprising SEQ ID NO: 30, and a CDR_{L3} having an amino acid sequence comprising SEQ ID NO: 41.

58. The antibody or antigen-binding fragment of claim 3, wherein the antibody or antigen-binding fragment comprises:
(a) an immunoglobulin heavy chain variable region comprising a CDR_{H1} having an amino acid sequence comprising SEQ ID NO: 1, a CDR_{H2} having an amino acid sequence comprising SEQ ID NO: 8, and a CDR_{H3} having an amino acid sequence comprising SEQ ID NO: 15; and
(b) an immunoglobulin light chain variable region comprising a CDR_{L1} having an amino acid sequence comprising SEQ ID NO: 22, a CDR_{L2} having an amino acid sequence comprising SEQ ID NO: 29, and a CDR_{L3} having an amino acid sequence comprising SEQ ID NO: 35.

59. The antibody or antigen-binding fragment of claim 3, wherein the antibody or antigen-binding fragment comprises:
(a) an immunoglobulin heavy chain variable region comprising a CDR_{H1} having an amino acid sequence comprising any one of SEQ ID NO: 2, a CDR_{H2} having an amino acid sequence comprising SEQ ID NO: 9 and a CDR_{H3} having an amino acid sequence comprising SEQ ID NO: 16; and
(b) an immunoglobulin light chain variable region comprising a CDR_{L1} having an amino acid sequence comprising SEQ ID NO: 23 a CDR_{L2} having an amino acid sequence comprising SEQ ID NO: 30, and a CDR_{L3} having an amino acid sequence comprising SEQ ID NO: 36.

60. The antibody or antigen-binding fragment of claim 3, wherein the antibody or antigen-binding fragment comprises:
(a) an immunoglobulin heavy chain variable region comprising a CDR_{H1} having an amino acid sequence comprising SEQ ID NO: 3, a CDR_{H2} having an amino acid sequence comprising SEQ ID NO: 10, and a CDR_{H3} having an amino acid sequence comprising SEQ ID NO: 17; and
(b) an immunoglobulin light chain variable region comprising a CDR_{L1} having an amino acid sequence comprising SEQ ID NO: 24, a CDR_{L2} having an amino acid sequence comprising SEQ ID NO: 31, and a CDR_{L3} having an amino acid sequence comprising SEQ ID NO: 37.

61. The antibody or antigen-binding fragment of claim 3, wherein the antibody or antigen-binding fragment comprises:
(a) an immunoglobulin heavy chain variable region comprising a CDR_{H1} having an amino acid sequence comprising SEQ ID NO: 4, a CDR_{H2} having an amino acid sequence comprising SEQ ID NO: 11, and a CDR_{H3} having an amino acid sequence comprising SEQ ID NO: 18; and
(b) an immunoglobulin light chain variable region comprising a CDR_{L1} having an amino acid sequence comprising SEQ ID NO: 25, a CDR_{L2} having an amino acid sequence comprising SEQ ID NO: 32, and a CDR_{L3} having an amino acid sequence comprising SEQ ID NO: 38.

62. The antibody or antigen-binding fragment of claim 3, wherein the antibody or antigen-binding fragment comprises:
(a) an immunoglobulin heavy chain variable region comprising a CDR_{H1} having an amino acid sequence comprising SEQ ID NO: 5, a CDR_{H2} having an amino acid sequence comprising SEQ ID NO: 12, and a CDR_{H3} having an amino acid sequence comprising SEQ ID NO: 19; and
(b) an immunoglobulin light chain variable region comprising a CDR_{L1} having an amino acid sequence comprising SEQ ID NO: 26, a CDR_{L2} having an amino acid sequence comprising SEQ ID NO: 33, and a CDR_{L3} having an amino acid sequence comprising SEQ ID NO: 39.

63. The antibody or antigen-binding fragment of claim 3, wherein the antibody or antigen-binding fragment comprises:
(a) an immunoglobulin heavy chain variable region comprising a CDR_{H1} having an amino acid sequence comprising SEQ ID NO: 6, a CDR_{H2} having an amino acid sequence comprising SEQ ID NO: 13, and a CDR_{H3} having an amino acid sequence comprising SEQ ID NO: 20; and
(b) an immunoglobulin light chain variable region comprising a CDR_{L1} having an amino acid sequence comprising SEQ ID NO: 27, a CDR_{L2} having an amino acid sequence comprising SEQ ID NO: 34, and a CDR_{L3} having an amino acid sequence comprising SEQ ID NO: 40.

64. The antibody or antigen-binding fragment of claim 3, wherein the antibody or antigen-binding fragment comprises:
(a) an immunoglobulin heavy chain variable region comprising a CDR_{H1} having an amino acid sequence comprising SEQ ID NO: 7, a CDR_{H2} having an amino acid sequence comprising SEQ ID NO: 14, and a CDR_{H3} having an amino acid sequence comprising SEQ ID NO: 21; and
(b) an immunoglobulin light chain variable region comprising a CDR_{L1} having an amino acid sequence comprising SEQ ID NO: 28, a CDR_{L2} having an amino acid sequence comprising SEQ ID NO: 30, and a CDR_{L3} having an amino acid sequence comprising SEQ ID NO: 41.

65. The antibody or antigen-binding fragment of claim 3, wherein the antibody or antigen-binding fragment comprises an immunoglobulin heavy chain variable region comprising an amino acid sequence having at least about 70% identity to any one of SEQ ID NOs: 93-99.

66.-77. (canceled)

78. The antibody or antigen-binding fragment of claim 3, wherein the antibody or antigen-binding fragment comprises an immunoglobulin light chain variable region comprising an amino acid sequence having at least about 70% identity to SEQ ID NOs: 100-106.

79.-90. (canceled)

91. The antibody or antigen-binding fragment of claim 3, wherein the antibody or antigen-binding fragment comprises an immunoglobulin heavy chain variable region comprising an amino acid sequence having at least about 70%, at least about 75%, at least about 80%, at least about 85%, at least about 90%, at least about 95%, at least about 96%, at least about 97%, at least about 98%, at least about 99%, at least about 99.5%, or at least about 99.9% sequence identity to any one of SEQ ID NOs: 93-99 and an immunoglobulin light chain variable region comprising an amino acid sequence having at least about 70%, at least about 75%, at least about 80%, at least about 85%, at least about 90%, at least about 95%, at least about 96%, at least about 97%, at least about 98%, at least about 99%, at least about 99.5% or at least about 99.9% identity to any one of SEQ ID NOs: 100-106.

92.-106. (canceled)

107. The antibody or antigen-binding fragment of claim 3, wherein the antibody or antigen-binding fragment comprises an immunoglobulin heavy chain variable region comprising SEQ ID NO: 93 and an immunoglobulin light chain variable region comprising SEQ ID NO: 100.

108. The antibody or antigen-binding fragment of claim 3, wherein the antibody or antigen-binding fragment comprises an immunoglobulin heavy chain variable region comprising SEQ ID NO: 94 and an immunoglobulin light chain variable region comprising SEQ ID NO: 101.

109. The antibody or antigen-binding fragment of claim 3, wherein the antibody or antigen-binding fragment comprises an immunoglobulin heavy chain variable region comprising SEQ ID NO: 95 and an immunoglobulin light chain variable region comprising SEQ ID NO: 102.

110. The antibody or antigen-binding fragment of claim 3, wherein the antibody or antigen-binding fragment comprises an immunoglobulin heavy chain variable region comprising SEQ ID NO: 96 and an immunoglobulin light chain variable region comprising SEQ ID NO: 103.

111. The antibody or antigen-binding fragment of claim 3, wherein the antibody or antigen-binding fragment comprises an immunoglobulin heavy chain variable region comprising SEQ ID NO: 97 and an immunoglobulin light chain variable region comprising SEQ ID NO: 104.

112. The antibody or antigen-binding fragment of claim 3, wherein the antibody or antigen-binding fragment comprises an immunoglobulin heavy chain variable region comprising SEQ ID NO: 98 and an immunoglobulin light chain variable region comprising SEQ ID NO: 105.

113. The antibody or antigen-binding fragment of claim 3, wherein the antibody or antigen-binding fragment comprises an immunoglobulin heavy chain variable region comprising SEQ ID NO: 99 and an immunoglobulin light chain variable region comprising SEQ ID NO: 106.

114.-140. (canceled)

141. A pharmaceutical composition for preventing or treating an influenza infection, the composition comprising an antibody or antigen-binding fragment of claim 3.

142.-156. (canceled)


SEQUENCE LISTING (163 sequences)

SEQ ID NO: 1 - protein, 8 aa, Homo sapiens
GDSIGGSY

SEQ ID NO: 2 - protein, 8 aa, Homo sapiens
GYTFINHA

SEQ ID NO: 3 - protein, 10 aa, Homo sapiens
GGSISSGGNY

SEQ ID NO: 4 - protein, 10 aa, Homo sapiens
GDSISGSSYY

SEQ ID NO: 5 - protein, 8 aa, Homo sapiens
GFSFSAYG

SEQ ID NO: 6 - protein, 8 aa, Homo sapiens
GVTLDNYW

SEQ ID NO: 7 - protein, 8 aa, Homo sapiens
GFPFSHYY

SEQ ID NO: 8 - protein, 7 aa, Homo sapiens
IYYTGIT

SEQ ID NO: 9 - protein, 8 aa, Homo sapiens
IIPIFGLA

SEQ ID NO: 10 - protein, 7 aa, Homo sapiens
ISYSGST

SEQ ID NO: 11 - protein, 7 aa, Homo sapiens
IYYSGIT

SEQ ID NO: 12 - protein, 8 aa, Homo sapiens
LGYDGTDQ

SEQ ID NO: 13 - protein, 8 aa, Homo sapiens
INADGTSK

SEQ ID NO: 14 - protein, 8 aa, Homo sapiens
IYSDGTSS

SEQ ID NO: 15 - protein, 19 aa, Homo sapiens
ARGDYSGYDRDVQVELMDV

SEQ ID NO: 16 - protein, 23 aa, Homo sapiens
ARDTVAVYEDFDWSSPYFFYMDV

SEQ ID NO: 17 - protein, 20 aa, Homo sapiens
ARGRGYCSRGATCYNFYMDV

SEQ ID NO: 18 - protein, 12 aa, Homo sapiens
ARLYTKSSNANY

SEQ ID NO: 19 - protein, 25 aa, Homo sapiens
ARGARPYYTDYRDHRPSYFYYHMDV

SEQ ID NO: 20 - protein, 13 aa, Homo sapiens
ARGGLYSSDAFDV

SEQ ID NO: 21 - protein, 13 aa, Homo sapiens
CRGGYYSLDGFDF

SEQ ID NO: 22 - protein, 6 aa, Homo sapiens
QTISIF

SEQ ID NO: 23 - protein, 7 aa, Homo sapiens
QSAGSKS

SEQ ID NO: 24 - protein, 9 aa, Homo sapiens
SGDIGGYNL

SEQ ID NO: 25 - protein, 6 aa, Homo sapiens
QSISSW

SEQ ID NO: 26 - protein, 8 aa, Homo sapiens
SSNIGNNY

SEQ ID NO: 27 - protein, 9 aa, Homo sapiens
SWDVGRYNH

SEQ ID NO: 28 - protein, 6 aa, Homo sapiens
QGIGND

SEQ ID NO: 29 - protein, 3 aa, Homo sapiens
AAS

SEQ ID NO: 30 - protein, 3 aa, Homo sapiens
GAS

SEQ ID NO: 31 - protein, 3 aa, Homo sapiens
EDS

SEQ ID NO: 32 - protein, 3 aa, Homo sapiens
DAS

SEQ ID NO: 33 - protein, 3 aa, Homo sapiens
DSD

SEQ ID NO: 34 - protein, 3 aa, Homo sapiens
EVN

SEQ ID NO: 35 - protein, 9 aa, Homo sapiens
QQSYSAPWT

SEQ ID NO: 36 - protein, 9 aa, Homo sapiens
QRYGTSLVT

SEQ ID NO: 37 - protein, 8 aa, Homo sapiens
CSHAGSVV

SEQ ID NO: 38 - protein, 9 aa, Homo sapiens
QQYHSYSGT

SEQ ID NO: 39 - protein, 11 aa, Homo sapiens
GTWDNSLNVLV

SEQ ID NO: 40 - protein, 10 aa, Homo sapiens
SSYTGNNVAV

SEQ ID NO: 41 - protein, 9 aa, Homo sapiens
LQHSSFPYT

SEQ ID NO: 42 - protein, 25 aa, Homo sapiens
QVQLQESGPGLVRPSETLSLTCTVS

SEQ ID NO: 43 - protein, 25 aa, Homo sapiens
EVQLVQSGAEVKKPGSSVKVSCKAS

SEQ ID NO: 44 - protein, 25 aa, Homo sapiens
QVQLQESGPGLVKPSQTLSLTCTVS

SEQ ID NO: 45 - protein, 25 aa, Homo sapiens
QVQLQESGPGLVKPSETLSLTCTVS

SEQ ID NO: 46 - protein, 25 aa, Homo sapiens
EVQLVESGGSVVQPGRSLRLSCAAF

SEQ ID NO: 47 - protein, 25 aa, Homo sapiens [sequence omitted]

SEQ ID NO: 48 - protein, 17 aa, Homo sapiens
WNWIRQPPGKGLQWIGY

SEQ ID NO: 49 - protein, 17 aa, Homo sapiens
LSWVRQAPGQGLEWVGG

SEQ ID NO: 50 - protein, 17 aa, Homo sapiens
WSWIRQHPGKGLEWIGF

SEQ ID NO: 51 - protein, 17 aa, Homo sapiens
WGWIRQPPGKGLEWIGS

SEQ ID NO: 52 - protein, 17 aa, Homo sapiens
MHWVRQAPGKGLEWVTL

SEQ ID NO: 53 - protein, 17 aa, Homo sapiens
VHWVRQVPGKGLVWVSR

SEQ ID NO: 54 - protein, 17 aa, Homo sapiens
MHWVRRAPGKGLVWVSR

SEQ ID NO: 55 - protein, 38 aa, Homo sapiens
NYNPSLKSRVTMSLDTSKNQISLKMDSVTAADTALYFC

SEQ ID NO: 56 - protein, 38 aa, Homo sapiens
KYGQKFQDRVTITADESTKTAYMDLRSLRSDDTAVYYC

SEQ ID NO: 57 - protein, 38 aa, Homo sapiens
YFTPSLNSRLTISVDTTNNHFSLKLSSVTGADTAVYYC

SEQ ID NO: 58 - protein, 38 aa, Homo sapiens
YYNPSLKSRVTIYVDTSKNQFSLKLNSATAADTAVYYC

SEQ ID NO: 59 - protein, 38 aa, Homo sapiens
WVAESVKGRFTVSRDNSRNTVILQMDSLRAEDTAVYFC

SEQ ID NO: 60 - protein, 38 aa, Homo sapiens
TYADSVKGRFTISRDTTRNTLFLQMNSLRGDDTALYFC

SEQ ID NO: 61 - protein, 38 aa, Homo sapiens
SYGDSVKGRFTISRDNAKNILYLQMNSLRAEDSATYYC

SEQ ID NO: 62 - protein, 11 aa, Homo sapiens
WGKGTTVTVSS

SEQ ID NO: 63 - protein, 11 aa, Homo sapiens
WGVGTTVTVSS

SEQ ID NO: 64 - protein, 11 aa, Homo sapiens
WGQGTLVTVSS

SEQ ID NO: 65 - protein, 11 aa, Homo sapiens
WGAGTTVTVSS

SEQ ID NO: 66 - protein, 11 aa, Homo sapiens
WGQGTMVTVSS

SEQ ID NO: 67 - protein, 26 aa, Homo sapiens
DIQMTQSPSSLSASVRDKVTFVCRAS

SEQ ID NO: 68 - protein, 26 aa, Homo sapiens
EIVLTQSPATLSLFPGERATLSCRAS

SEQ ID NO: 69 - protein, 25 aa, Homo sapiens
QSALTQPASVSGSPGQSITISCTGT

SEQ ID NO: 70 - protein, 26 aa, Homo sapiens
DIQMTQSPSTLSASVGDRVTITCRAS

SEQ ID NO: 71 - protein, 25 aa, Homo sapiens
QSVLTQPPSVSAAPGQKVTISCSGS

SEQ ID NO: 72 - protein, 25 aa, Homo sapiens
QSALTQPPSASGSPGQSVTISCTGT

SEQ ID NO: 73 - protein, 26 aa, Homo sapiens
DIQMTQSPLSLSVSEGDRVTITCRAS

SEQ ID NO: 74 - protein, 17 aa, Homo sapiens
LNWYQHKPGEAPKLLIY

SEQ ID NO: 75 - protein, 17 aa, Homo sapiens
LAWYQHKVGQPPRLLIN

SEQ ID NO: 76 - protein, 17 aa, Homo sapiens
VSWYQHHPGRVPKLIIY

SEQ ID NO: 77 - protein, 17 aa, Homo sapiens
LAWYQQKPGKAPKLLIY

SEQ ID NO: 78 - protein, 17 aa, Homo sapiens
VSWYQQLPGTAPKLLIY

SEQ ID NO: 79 - protein, 17 aa, Homo sapiens
VSWYQHHPGKAHKLIIY

SEQ ID NO: 80 - protein, 17 aa, Homo sapiens
LGWYQLKPGKAPKRLIY

SEQ ID NO: 81 - protein, 36 aa, Homo sapiens
RLQSGVPSRFSGSGSGTDFTLTISGLQPEDFATYYC

SEQ ID NO: 82 - protein, 36 aa, Homo sapiens
SRATGIPDRFSGSGSGPDFNLTISRLEPEDFAVYYC

SEQ ID NO: 83 - protein, 36 aa, Homo sapiens
KRPSGFLNRFSGSKSGNTASLTISGLQAEDVADYYC

SEQ ID NO: 84 - protein, 36 aa, Homo sapiens
SLESGVPSRFSGSGSGTEFTLTISSLQSDDFAIYYC

SEQ ID NO: 85 - protein, 36 aa, Homo sapiens
KRPSGIPARFSGSKSGASATLAITGLQTGDEADYYC

SEQ ID NO: 86 - protein, 36 aa, Homo sapiens
RRPSGVPDRFSGSKSANTASLTVSGLQAEDEADYYC

SEQ ID NO: 87 - protein, 36 aa, Homo sapiens
SLQSGVPSRFSGSGSGTEFTLTISSLQPEDFATYFC

SEQ ID NO: 88 - protein, 10 aa, Homo sapiens
FGQGTKVEIK

SEQ ID NO: 89 - protein, 10 aa, Homo sapiens
FGGGTKVEIK

SEQ ID NO: 90 - protein, 10 aa, Homo sapiens
FGGGTRLTVQ

SEQ ID NO: 91 - protein, 10 aa, Homo sapiens
FGGGTKLAVL

SEQ ID NO: 92 - protein, 10 aa, Homo sapiens
FGGGTKLTVL

SEQ ID NO: 93 - protein, 125 aa, Homo sapiens [sequence omitted]

SEQ ID NO: 94 - protein, 130 aa, Homo sapiens
EVQLVQSGAEVKKPGSSVKVSCKASGYTFINHALSWVRQAPGQGLEWVGGIIPIFGLAKYGQKFQDRVTITADESTKTAYMDLRSLRSDDTAVYYCARDTVAVYEDFDWSSPYFFYMDVWGKGTTVTVSS

SEQ ID NO: 95 - protein, 128 aa, Homo sapiens
QVQLQESGPGLVKPSQTLSLTCTVSGGSISSGGNYWSWIRQHPGKGLEWIGFISYSGSTYFTPSLNSRLTISVDTTNNHFSLKLSSVTGADTAVYYCARGRGYCSRGATCYNFYMDVWGVGTTVTVSS

SEQ ID NO: 96 - protein, 120 aa, Homo sapiens
QVQLQESGPGLVKPSETLSLTCTVSGDSISGSSYYWGWIRQPPGKGLEWIGSIYYSGITYYNPSLKSRVTIYVDTSKNQFSLKLNSATAADTAVYYCARLYTKSSNANYWGQGTLVTVSS

SEQ ID NO: 97 - protein, 132 aa, Homo sapiens
EVQLVESGGSVVQPGRSLRLSCAAFGFSFSAYGMHWVRQAPGKGLEWVTLLGYDGTDQWVAESVKGRFTVSRDNSRNTVILQMDSLRAEDTAVYFCARGARPYYTDYRDHRPSYFYYHMDVWGAGTTVTVSS

SEQ ID NO: 98 - protein, 120 aa, Homo sapiens
EVQLVESGGGLVQPGGSLRLSCAASGVTLDNYWVHWVRQVPGKGLVWVSRINADGTSKTYADSVKGRFTISRDTTRNTLFLQMNSLRGDDTALYFCARGGLYSSDAFDVWGQGTMVTVSS

SEQ ID NO: 99 - protein, 120 aa, Homo sapiens
EVQLVESGGGLVQPGGSLRLSCAASGFPFSHYYMHWVRRAPGKGLVWVSRIYSDGTSSSYGDSVKGRFTISRDNAKNILYLQMNSLRAEDSATYYCCRGGYYSLDGFDFWGQGTMVTVSS

SEQ ID NO: 100 - protein, 107 aa, Homo sapiens
DIQMTQSPSSLSASVRDKVTFVCRASQTISIFLNWYQHKPGEAPKLLIYAASRLQSGVPSRFSGSGSGTDFTLTISGLQPEDFATYYCQQSYSAPWTFGQGTKVEIK

SEQ ID NO: 101 - protein, 108 aa, Homo sapiens
EIVLTQSPATLSLFPGERATLSCRASQSAGSKSLAWYQHKVGQPPRLLINGASSRATGIPDRFSGSGSGPDFNLTISRLEPEDFAVYYCQRYGTSLVTFGGGTKVEIK

SEQ ID NO: 102 - protein, 108 aa, Homo sapiens
QSALTQPASVSGSPGQSITISCTGTSGDIGGYNLVSWYQHHPGRVPKLIIYEDSKRPSGFLNRFSGSKSGNTASLTISGLQAEDVADYYCCSHAGSVVFGGGTRLTVQ

SEQ ID NO: 103 - protein, 107 aa, Homo sapiens [sequence omitted]

SEQ ID NO: 104 - protein, 110 aa, Homo sapiens
QSVLTQPPSVSAAPGQKVTISCSGSSSNIGNNYVSWYQQLPGTAPKLLIYDSDKRPSGIPARFSGSKSGASATLAITGLQTGDEADYYCGTWDNSLNVLVFGGGTKLAVL

SEQ ID NO: 105 - protein, 110 aa, Homo sapiens
QSALTQPPSASGSPGQSVTISCTGTSWDVGRYNHVSWYQHHPGKAHKLIIYEVNRRPSGVPDRFSGSKSANTASLTVSGLQAEDEADYYCSSYTGNNVAVFGGGTKLTVL

SEQ ID NO: 106 - protein, 107 aa, Homo sapiens
DIQMTQSPLSLSVSEGDRVTITCRASQGIGNDLGWYQLKPGKAPKRLIYGASSLQSGVPSRFSGSGSGTEFTLTISSLQPEDFATYFCLQHSSFPYTFGQGTKVEIK

SEQ ID NO: 107 - protein, 97 aa, Homo sapiens
QVQLQESGPGLVRPSETLSLTCTVSGDSIGGSYWNWIRQPPGKGLQWIGYIYYTGITNYNPSLKSRVTMSLDTSKNQISLKMDSVTAADTALYFCAR

SEQ ID NO: 108 - protein, 98 aa, Homo sapiens
EVQLVQSGAEVKKPGSSVKVSCKASGYTFINHALSWVRQAPGQGLEWVGGIIPIFGLAKYGQKFQDRVTITADESTKTAYMDLRSLRSDDTAVYYCAR

SEQ ID NO: 109 - protein, 99 aa, Homo sapiens
QVQLQESGPGLVKPSQTLSLTCTVSGGSISSGGNYWSWIRQHPGKGLEWIGFISYSGSTYFTPSLNSRLTISVDTTNNHFSLKLSSVTGADTAVYYCAR

SEQ ID NO: 110 - protein, 99 aa, Homo sapiens
QVQLQESGPGLVKPSETLSLTCTVSGDSISGSSYYWGWIRQPPGKGLEWIGSIYYSGITYYNPSLKSRVTIYVDTSKNQFSLKLNSATAADTAVYYCAR

SEQ ID NO: 111 - protein, 98 aa, Homo sapiens
EVQLVESGGSVVQPGRSLRLSCAAFGFSFSAYGMHWVRQAPGKGLEWVTLLGYDGTDQWVAESVKGRFTVSRDNSRNTVILQMDSLRAEDTAVYFCAR

SEQ ID NO: 112 - protein, 98 aa, Homo sapiens
EVQLVESGGGLVQPGGSLRLSCAASGVTLDNYWVHWVRQVPGKGLVWVSRINADGTSKTYADSVKGRFTISRDTTRNTLFLQMNSLRGDDTALYFCAR

SEQ ID NO: 113 - protein, 96 aa, Homo sapiens
EVQLVESGGGLVQPGGSLRLSCAASGFPFSHYYMHWVRRAPGKGLVWVSRIYSDGTSSSYGDSVKGRFTISRDNAKNILYLQMNSLRAEDSATYYC

SEQ ID NO: 114 - protein, 94 aa, Homo sapiens
DIQMTQSPSSLSASVRDKVTFVCRASQTISIFLNWYQHKPGEAPKLLIYAASRLQSGVPSRFSGSGSGTDFTLTISGLQPEDFATYYCQQSYSA

SEQ ID NO: 115 - protein, 95 aa, Homo sapiens
EIVLTQSPATLSLFPGERATLSCRASQSAGSKSLAWYQHKVGQPPRLLINGASSRATGIPDRFSGSGSGPDFNLTISRLEPEDFAVYYCQRYGTS

SEQ ID NO: 116 - protein, 98 aa, Homo sapiens
QSALTQPASVSGSPGQSITISCTGTSGDIGGYNLVSWYQHHPGRVPKLIIYEDSKRPSGFLNRFSGSKSGNTASLTISGLQAEDVADYYCCSHAGSVV

SEQ ID NO: 117 - protein, 94 aa, Homo sapiens
DIQMTQSPSTLSASVGDRVTITCRASQSISSWLAWYQQKPGKAPKLLIYDASSLESGVPSRFSGSGSGTEFTLTISSLQSDDFAIYYCQQYHSY

SEQ ID NO: 118 - protein, 97 aa, Homo sapiens
QSVLTQPPSVSAAPGQKVTISCSGSSSNIGNNYVSWYQQLPGTAPKLLIYDSDKRPSGIPARFSGSKSGASATLAITGLQTGDEADYYCGTWDNSLN

SEQ ID NO: 119 - protein, 97 aa, Homo sapiens
QSALTQPPSASGSPGQSVTISCTGTSWDVGRYNHVSWYQHHPGKAHKLIIYEVNRRPSGVPDRFSGSKSANTASLTVSGLQAEDEADYYCSSYTGNN

SEQ ID NO: 120 - protein, 94 aa, Homo sapiens
DIQMTQSPLSLSVSEGDRVTITCRASQGIGNDLGWYQLKPGKAPKRLIYGASSLQSGVPSRFSGSGSGTEFTLTISSLQPEDFATYFCLQHSSF

SEQ ID NO: 121 - protein, 21 aa, Homo sapiens
CARGDYSGYDRDVQVELMDVW

SEQ ID NO: 122 - protein, 25 aa, Homo sapiens
CARDTVAVYEDFDWSSPYFFYMDVW

SEQ ID NO: 123 - protein, 22 aa, Homo sapiens
CARGRGYCSRGATCYNFYMDVW

SEQ ID NO: 124 - protein, 14 aa, Homo sapiens
CARLYTKSSNANYW

SEQ ID NO: 125 - protein, 27 aa, Homo sapiens
CARGARPYYTDYRDHRPSYFYYHMDVW

SEQ ID NO: 126 - protein, 15 aa, Homo sapiens
CARGGLYSSDAFDVW

SEQ ID NO: 127 - protein, 15 aa, Homo sapiens
CCRGGYYSLDGFDFW

SEQ ID NO: 128 - protein, 11 aa, Homo sapiens [sequence omitted]

SEQ ID NO: 129 - protein, 11 aa, Homo sapiens
CQRYGTSLVTF

SEQ ID NO: 130 - protein, 10 aa, Homo sapiens
CCSHAGSVVF

SEQ ID NO: 131 - protein, 11 aa, Homo sapiens [sequence omitted]

SEQ ID NO: 132 - protein, 13 aa, Homo sapiens
CGTWDNSLNVLVF

SEQ ID NO: 133 - protein, 12 aa, Homo sapiens
CSSYTGNNVAVF

SEQ ID NO: 134 - protein, 11 aa, Homo sapiens
CLQHSSFPYTF

SEQ ID NO: 135 - protein, 14 aa, Homo sapiens
MDVWGKGTTVTVSS

SEQ ID NO: 136 - protein, 18 aa, Homo sapiens
YFFYMDVWGKGTTVTVSS

SEQ ID NO: 137 - protein, 15 aa, Homo sapiens
YMDVWGVGTTVTVSS

SEQ ID NO: 138 - protein, 12 aa, Homo sapiens
YWGQGTLVTVSS

SEQ ID NO: 139 - protein, 17 aa, Homo sapiens
YYHMDVWGAGTTVTVSS

SEQ ID NO: 140 - protein, 16 aa, Homo sapiens
DAFDVWGQGTMVTVSS

SEQ ID NO: 141 - protein, 16 aa, Homo sapiens
DGFDFWGQGTMVTVSS

SEQ ID NO: 142 - protein, 12 aa, Homo sapiens
WTFGQGTKVEIK

SEQ ID NO: 143 - protein, 11 aa, Homo sapiens
TFGGGTKVEIK

SEQ ID NO: 144 - protein, 9 aa, Homo sapiens
GGGTRLTVQ

SEQ ID NO: 145 - protein, 11 aa, Homo sapiens
TFGQGTKVEIK

SEQ ID NO: 146 - protein, 11 aa, Homo sapiens
VFGGGTKLAVL

SEQ ID NO: 147 - protein, 12 aa, Homo sapiens
AVFGGGTKLTVL

SEQ ID NO: 148 - protein, 12 aa, Homo sapiens
YTFGQGTKVEIK

SEQ ID NO: 149 - protein, 387 aa, Influenza B virus [sequence omitted]

SEQ ID NO: 150 - DNA, 375 bp, Homo sapiens
caggtgcagctgcaggagtcgggcccaggactggtgaggccttcggagaccctgtccctcacctgcactgtctctggtgactccatcggtggtagctactggaactggatccggcagcccccagggaagggactgcagtggattggctacatttactatactgggatcaccaactacaacccctccctcaagagccgagtcaccatgtcactcgacacgtccaagaaccagatctccctgaaaatggactctgtgaccgctgcggacacggccctttatttctgtgcgagaggtgactatagtggctacgatcgggatgtgcaagtggaactcatggacgtctggggcaaagggaccacggtcaccgtctcctca

SEQ ID NO: 151 - DNA, 390 bp, Homo sapiens
gaggtgcagctggtgcaatctggggctgaggtgaagaagcctgggtcctcggtgaaggtctcctgcaaggcttctggatacaccttcatcaatcatgctctcagctgggtgcgacaggcccctgggcaagggcttgagtgggtgggagggatcatccctatctttggtctggcgaagtatggacaaaagttccaggacagagtcacgattaccgcggacgaatccacgaagacagcctacatggacctgagaagcctgagatctgacgacacggccgtttattattgtgcgagagacactgttgcggtatacgaggattttgactggtcatcaccatacttcttctacatggacgtctggggcaaagggaccacggtcaccgtctcctca

SEQ ID NO: 152 - DNA, 384 bp, Homo sapiens
caggtgcagctgcaggagtcgggtccaggactggtgaagccttcacagaccctgtccctcacctgcactgtctctggtggctccatcagcagtggtggtaactactggagctggatccgtcaacacccagggaagggcctggagtggattgggttcatctcttacagtgggagtacctacttcactccgtccctcaacagccgactgaccatatcagtagacacgactaacaaccacttctccctgaagctgagctctgtgactggcgcggacacggccgtttattactgtgcgagaggacggggatattgtagtagaggtgccacgtgctacaatttctacatggacgtctggggcgtagggaccacggtcaccgtctcctca

SEQ ID NO: 153 - DNA, 360 bp, Homo sapiens
caggtgcagctgcaggagtcgggcccaggactggtgaagccttcggagaccctgtccctcacctgcactgtctctggcgactccatcagcggtagtagttattactggggctggatccgccagcccccagggaaggggctggagtggattgggagtatctattatagtgggatcacctactacaacccgtccctcaagagtcgagtcaccatatacgttgacacgtccaagaaccagttctccctgaagctgaactctgcgaccgccgcagacacggctgtgtattattgtgcgagactatataccaagagctcaaacgccaactactggggccagggaaccctggtcaccgtctcctca

SEQ ID NO: 154 - DNA, 396 bp, Homo sapiens
gaagtgcagctggtggagtctgggggaagcgtggtccagcctgggaggtccctgagactctcctgtgcagcctttggattcagcttcagtgcatatggcatgcactgggtccgccaggctccaggcaaggggctggagtgggtgacacttctagggtatgatgggactgatcaatgggttgcagagtccgtgaagggccgattcaccgtctccagagacaactccaggaacacagtaattctgcaaatggacagcctgagagccgaggacacggctgtttatttctgtgcgagaggagcgcgcccctactacactgactacagggatcaccgaccctcctacttctactatcacatggacgtctggggcgctgggaccacggtcaccgtctcctca

SEQ ID NO: 155 - DNA, 361 bp, Homo sapiens
gaggtgcagctggtggagtccgggggaggcttagttcagccgggggggtccctgagactctcctgtgcagcctctggagtcaccctcgataactattgggtacactgggtccgccaagttccagggaaggggctggtgtgggtctcacgcattaatgctgatgggaccagtaaaacatacgcggactccgtgaagggccgattcaccatctccagagacaccaccaggaacactctgtttctacaaatgaacagtctgagaggcgacgacacggctctgtatttttgtgcgagaggcgggctgtacagtagtgatgcctttgatgtttggggccaagggacaatggtcaccgtctcttcag

SEQ ID NO: 156 - DNA, 361 bp, Homo sapiens
gaggtgcagctggtggagtccgggggaggcttagttcagcctggggggtccctgagactctcctgtgcagcctctggattccccttcagtcactactacatgcactgggtccgccgagctccagggaaggggctggtttgggtctcacgtatttacagtgatgggactagttccagttacggggactccgtgaagggccgattcaccatctccagagacaacgccaagaacattctgtatctgcaaatgaacagtctgagagccgaagactcggctacttactactgttgccggggtggttattattctttggatggttttgatttctggggccaagggacaatggtcaccgtctcttcag

SEQ ID NO: 157 - DNA, 322 bp, Homo sapiens
gacatccagatgacccagtctccatcctccctgtctgcatctgtgcgagacaaagtcaccttcgtttgccgggcaagtcagaccataagcatctttttaaattggtatcaacacaaaccaggggaagcccccaagctcctgatctatgctgcgtccaggttgcaaagtggggtcccatcaaggttcagtggcagtgggtctgggacagatttcactctcaccatcagcggtctgcagcctgaggattttgcaacttactactgtcaacagagttacagtgccccgtggacgttcggccaagggaccaaggtggaaatcaaac

SEQ ID NO: 158 - DNA, 325 bp, Homo sapiens
gaaattgtgttgacgcagtctccagccaccctgtctctgtttccaggggagagagccaccctctcatgcagggccagtcagagtgctggcagcaagtccttagcctggtaccagcacaaagttggccagcctcccaggctcctcatcaatggtgcctccagcagggccactggcatcccagacaggttcagtggcagcgggtctgggccagacttcaatctaaccatcagcagactggagcctgaagattttgcagtgtattactgtcagcgatatggtacctcacttgtcaccttcggcggcgggaccaaggtggaaatcaaac

SEQ ID NO: 159 - DNA, 325 bp, Homo sapiens
cagtctgccctgactcagcctgcctccgtgtctgggtctcctggacagtcgatcaccatctcctgcactggaaccagcggtgatattgggggttataaccttgtctcctggtaccaacaccacccaggcagagtccccaaactcataatttatgaggacagtaaacggccctcagggtttttaaatcgcttctctggctccaagtctggcaacacggcctccctgacaatctctgggctccaggctgaggacgtggcagattattactgctgctcacatgcaggtagtgtggtcttcggcggagggaccaggctgaccgtccaag

SEQ ID NO: 160 - DNA, 322 bp, Homo sapiens
gacatccagatgacccagtctccttccaccctgtctgcatctgtaggagacagagtcaccatcacttgccgggccagtcagagtattagtagttggttggcctggtatcagcagaaaccagggaaagcccctaaactcctgatctatgacgcctccagtttggaaagtggggtcccatcaaggttcagcggcagtggatctgggacagaattcactctcaccatcagcagcctgcagtctgatgatttcgcaatttattactgccaacagtatcatagttattcagggacgttcggccaagggaccaaggtggaaatcaaac

SEQ ID NO: 161 - DNA, 331 bp, Homo sapiens
cagtctgtgctgacgcagccgccctcagtgtctgcggccccaggacagaaggtcaccatctcctgctctggaagcagctccaacattggtaataattatgtatcctggtaccaacaactcccaggaacagcccccaaactcctcatttatgacagtgataagcgaccctcagggattcctgcccgattctctggctccaagtctggcgcgtcagccaccctggccatcaccggactccagactggggacgaggccgattattactgcggaacatgggataatagcctgaatgttctagtattcggcggagggaccaagctggccgtcctag

SEQ ID NO: 162 - DNA, 331 bp, Homo sapiens
cagtctgccctgactcagcctccctccgcgtccgggtctcctggacagtcagtcaccatctcctgcactggaacgagttgggacgttggtcgttataaccatgtctcctggtaccaacaccacccaggcaaagcccacaaactcatcatttatgaggtcaataggcggccctcaggggtccctgatcgcttctctggctccaagtctgccaacacggcctccctgaccgtctctgggctccaggctgaggatgaggctgattattactgcagctcatacacaggcaacaacgttgccgtcttcggcggagggaccaagctgaccgtcctgc

SEQ ID NO: 163 - DNA, 322 bp, Homo sapiens
gacatccagatgacccagtctccactctccctgtctgtatctgaaggagacagagtcaccatcacttgccgggcaagtcagggcattggaaatgatttaggctggtatcaattgaaaccagggaaagcccctaagcgcctgatctatggtgcatccagtttgcaaagtggggtcccatcaaggttcagcggctctggatctgggacagaattcactctcacaatcagcagcctgcagcctgaagattttgcaacttacttctgtctacagcatagtagtttcccgtatacttttggccaggggaccaaggtggaaatcaaac